SEQUENCE LISTING (31 sequences)

SEQ ID NO: 1 - DNA, 1578 bp, synthetic construct
  misc_feature 1..1578: note = Ebola Sudan ancestor (T2-4), Unoptimised
  source 1..1578: mol_type = other DNA
atggggggtcttagcctactccaattgcccagggacaaatttcggaaaagctctttctttgtttgggtcatcatcttattccaaaaggccttttccatgcctttgggtgttgtgactaacagcactttagaagtaacagagattgaccagctagtctgcaaggatcatcttgcatccactgaccagctgaaatcagttggtctcaacctcgaggggagcggagtatctactgatatcccatctgcaacaaagcgttggggcttcagatctggtgttcctcccaaggtggtcagctatgaagcgggagaatgggctgaaaattgctacaatcttgaaataaagaagccggacgggagcgaatgcttacccccaccgccagatggtgtcagaggctttccaaggtgccgctatgttcacaaagcccaaggaaccgggccctgcccaggtgactacgcctttcacaaggatggagctttcttcctctatgacaggctggcttcaactgtaatttacagaggagtcaattttgctgagggggtaattgcattcttgatattggctaaaccaaaagaaacgttccttcagtcaccccccattcgagaggcagtaaactacactgaaaatacatcaagttattatgccacatcctacttggagtatgaaatcgaaaattttggtgctcaacactccacgacccttttcaaaattgacaataatacttttgttcgtctggacaggccccacacgcctcagttccttttccagctgaatgataccattcaccttcaccaacagttgagcaacacaactgggagactaatttggacactagatgctaatatcaatgctgatattggtgaatgggctttttgggaaaataaaaaaaatctctccgaacaactacgtggagaagagctgtctttcgaagctttatcgctcacaacagcggttaaaactgtcttgccacaggagtccacaagcaacggtctaataacttcaacagtaacagggattcttgggagtcttgggcttcgaaaacgcagcagaagacaagttaacaccaaagccacgggtaaatgcaatcccaacttacactactggactgcacaagaacaacataatgctgctgggattgcctggatcccgtactttggaccgggtgcggaaggcatatacactgaaggcctgatgcataaccaaaatgccttagtctgtggacttaggcaacttgcaaatgaaacaactcaagctctgcagcttttcttaagagccacaacggagctgcggacatataccatactcaataggaaggccatagatttccttctgcgacgatggggcgggacatgcaggatcctgggaccagattgttgcattgagccacatgattggacaaaaaacatcactgataaaatcaaccaaatcatccatgatttcatcgacaaccccttacctaatcaggataatgatgataattggtggacgggctggagacagtggatccctgcaggaataggcattactggaattattattgcaattattgctcttctttgcgtttgcaagctgctttgctag

SEQ ID NO: 2 - DNA, 1578 bp, synthetic construct
  misc_feature 1..1578: note = Ebola Sudan ancestor (T2-4); Gene-optimised
  source 1..1578: mol_type = other DNA
atgggaggactgtctctgctgcaactgccccgggacaagttccggaagtccagcttcttcgtgtgggtcatcatcctgttccagaaagccttcagcatgcccctgggcgtcgtgaccaatagcacactggaagtgaccgagatcgaccagctcgtgtgcaaggatcacctggccagcaccgatcagctgaagtctgtgggactgaatctggaaggcagcggcgtgtccacagatatccctagcgccaccaagagatggggctttagaagcggagtgcctcctaaggtggtgtcttatgaagccggcgagtgggccgagaactgctacaacctggaaatcaagaagcccgacggcagcgagtgtctgcctcctccacctgatggcgtcagaggcttccctagatgcagatacgtgcacaaggcccaaggcacaggaccctgtcctggcgattacgcctttcacaaggacggcgcctttttcctgtacgatcggctggcctccaccgtgatctacagaggcgttaactttgccgagggcgtgatcgccttcctgatcctggccaagcctaaagagacattcctgcaaagccctccaatccgcgaggccgtgaactacacagagaacaccagcagctactacgccaccagctacctggaatacgagatcgagaatttcggcgcccagcacagcaccacactgttcaagatcgacaacaacaccttcgtgcggctggacagaccccacacacctcagtttctgttccagctgaacgacaccatccatctgcatcagcagctgagcaacaccaccggcagactgatttggaccctggacgccaacatcaacgccgacattggagagtgggccttttgggagaacaagaagaacctgagcgaacagctgagaggcgaggaactgagctttgaggccctgtctctgaccaccgccgtgaaaacagtgctgcctcaagagtccaccagcaacggcctgatcacaagcacagtgacaggcatcctgggcagcctgggcctgagaaaaaggtccagacggcaagtgaataccaaggccaccggcaagtgcaaccccaacctgcactattggacagcccaagagcagcacaatgccgccggaatcgcctggattccttattttggacctggcgccgagggcatctataccgagggactgatgcacaaccagaacgccctcgtgtgtggactgagacagctggccaatgagacaacacaggccctccagctgtttctgagagccaccaccgagctgagaacctacaccatcctgaaccggaaggccatcgactttctgctgagaagatggggcggcacctgtagaatcctgggacctgattgctgcatcgagccccacgactggaccaagaacatcaccgacaagatcaaccagatcatccacgacttcatcgacaaccctctgcctaaccaggacaacgacgacaattggtggacaggctggcggcagtggattcctgccggaattggcatcaccggcatcatcattgccattatcgccctgctgtgtgtgtgcaagctgctgtgttga

SEQ ID NO: 3 - protein, 525 aa, synthetic construct
  REGION 1..525: note = Ebola Sudan ancestor (T2-4)
MGGLSLLQLPRDKFRKSSFFVWVIILFQKAFSMPLGVVTNSTLEVTEIDQLVCKDHLASTDQLKSVGLNLEGSGVSTDIPSATKRWGFRSGVPPKVVSYEAGEWAENCYNLEIKKPDGSECLPPPPDGVRGFPRCRYVHKAQGTGPCPGDYAFHKDGAFFLYDRLASTVIYRGVNFAEGVIAFLILAKPKETFLQSPPIREAVNYTENTSSYYATSYLEYEIENFGAQHSTTLFKIDNNTFVRLDRPHTPQFLFQLNDTIHLHQQLSNTTGRLIWTLDANINADIGEWAFWENKKNLSEQLRGEELSFEALSLTTAVKTVLPQESTSNGLITSTVTGILGSLGLRKRSRRQVNTKATGKCNPNLHYWTAQEQHNAAGIAWIPYFGPGAEGIYTEGLMHNQNALVCGLRQLANETTQALQLFLRATTELRTYTILNRKAIDFLLRRWGGTCRILGPDCCIEPHDWTKNITDKINQIIHDFIDNPLPNQDNDDNWWTGWRQWIPAGIGITGIIIAIIALLCVCKLLC

SEQ ID NO: 4 - DNA, 1581 bp, synthetic construct
  misc_feature 1..1581: note = Ebolavirus global ancestor (T2-6) Unoptimised
  source 1..1581: mol_type = other DNA
atggggggtggatccagacttctccaattgccccgggaacgctttcggaaaacctcattctttgtttgggtaatcatcctattccaaaaagccttttccatgccattgggtgttgtaaccaacagcactctaaaagtaacagaaattgaccaattggtttgccgggacaaactttcatccacaagtcagctgaaatcagttgggctgaatctggaagggaatggagttgcaactgatgtcccatcagcaacaaaacgatggggcttccgatctggtgttcctcccaaggtggtcagctatgaagctggagaatgggctgaaaattgctacaatctggaaatcaagaagccagacgggagtgaatgcctacctccaccgccagacggtgtaagaggcttccccaggtgccgctatgtccacaaagttcaaggaacagggccgtgtcctggtgacttcgccttccacaaagatggagctttcttcctgtatgatagactggcttcaactgtcatttaccgagggacaacttttgctgaaggtgtcgttgcatttttgatcctgcccaaacctaaaaaggactttttccaatcacccccaatacgtgagccggtaaacaccacagaagatccatcaagttactacaccacatcaacacttagctatgagattgacaattttggggccaataaaactaaaactcttttcaaagttgacaatcacacttatgtgcaactagaccgaccacacacaccacagttccttgtccagctcaatgaaaccattcatacaaataaccgtctaagcaacaccacagggagactaatttggacattagatcctaaaattgataccgacattggtgagtgggccttctgggaaaataaaaaaaacttctccaaacaacttcgtggagaagagttgtctttcaaagctctatcaacaaaaactggagctaacgcagtagacactgacgaatcaagcaaacctggcctaattaccaacacagtaagaggggttgctgatttactgagcccttggagaagaaaaagaagacaagtcaacccaaacacaacaaataaatgcaacccaaacctacactattggacagcccaagatgaaggtgctgccgttggattagcctggatcccatacttcggaccagcagcagaaggcatttacactgaaggaataatgcataatcaaaatgggttaatctgtgggctgaggcagctggccaatgaaacgactcaagctcttcaattattcttgagggccacaacggagctgcggacttactctatactcaatagaaaagccattgatttccttctccaacgatggggaggaacatgccgcatcttaggaccagattgttgcattgagccacatgattggacaaaaaacattactgataaaattaaccaaatcatacatgattttattgacaaccctctaccagatcaggacgatgatgacaattggtggacaggctggagacaatggatccctgctggaattggaattactggagttataattgcaattatagctctactttgtatttgcaagtttctgtgttag

SEQ ID NO: 5 - DNA, 1581 bp, synthetic construct
  misc_feature 1..1581: note = Ebolavirus global ancestor (T2-6) gene optimised
  source 1..1581: mol_type = other DNA
atgggcggaggatctagactgctgcaactgcccagagagcggttcagaaagaccagcttcttcgtgtgggtcatcatcctgttccagaaagccttcagcatgcccctgggcgtcgtgaccaatagcaccctgaaagtgaccgagatcgaccagctcgtgtgcagagataagctgagcagcaccagccagctgaagtccgtgggactgaatctggaaggcaatggcgtggccacagatgtgcctagcgccaccaaaagatggggctttagaagcggcgtgccacctaaggtggtgtcttatgaagccggcgagtgggccgagaactgctacaacctggaaatcaagaagcccgacggcagcgagtgtctgcctcctccacctgatggcgtcagaggcttccctagatgcagatacgtgcacaaggtgcaaggcacaggcccctgtcctggcgatttcgcctttcacaaggacggcgcctttttcctgtacgatcggctggcctccaccgtgatctacagaggcacaacatttgccgaaggcgtggtggccttcctgatcctgcctaagcctaagaaggacttctttcagagccctcctatccgcgagcctgtgaacacaacagaggaccccagcagctactacaccaccagcacactgagctacgagatcgataacttcggcgccaacaagaccaagacactgttcaaggtggacaaccacacctacgtgcagctggacagaccccacacacctcagtttctggtgcagctgaacgagacaatccacaccaacaacagactgagcaacaccaccggcaggctgatctggaccctggatcctaagatcgacaccgacatcggagagtgggccttttgggagaacaagaagaacttcagcaagcagctgagaggcgaggaactgagctttaaggccctgagcaccaagacaggcgccaacgctgtggataccgatgagtctagcaagcccggcctgatcaccaacacagttagaggcgttgccgacctgctgagcccttggagaagaaagcggagacaagtgaaccccaataccaccaacaagtgcaaccctaacctgcactactggacagcccaggatgaaggcgctgctgttggactggcctggattccttattttggacctgccgccgagggcatctacacagagggaatcatgcacaaccagaatggcctgatctgcggcctgagacagctggccaatgagacaacacaggccctccagctgtttctgagagccaccaccgagctgagaacctacagcatcctgaaccggaaggccatcgactttctgctgcaaagatggggaggcacctgtagaatcctgggacctgattgctgcatcgagccccacgactggaccaagaacatcaccgacaagatcaaccagatcatccacgacttcatcgacaaccctctgcctgaccaggacgacgacgataattggtggacaggatggcggcagtggattcctgccggaatcggaatcacaggcgtgatcattgccattatcgccctgctgtgcatctgcaagtttctgtgctga

SEQ ID NO: 6 - protein, 526 aa, synthetic construct
  REGION 1..526: note = Ebolavirus global ancestor (T2-6)
MGGGSRLLQLPRERFRKTSFFVWVIILFQKAFSMPLGVVTNSTLKVTEIDQLVCRDKLSSTSQLKSVGLNLEGNGVATDVPSATKRWGFRSGVPPKVVSYEAGEWAENCYNLEIKKPDGSECLPPPPDGVRGFPRCRYVHKVQGTGPCPGDFAFHKDGAFFLYDRLASTVIYRGTTFAEGVVAFLILPKPKKDFFQSPPIREPVNTTEDPSSYYTTSTLSYEIDNFGANKTKTLFKVDNHTYVQLDRPHTPQFLVQLNETIHTNNRLSNTTGRLIWTLDPKIDTDIGEWAFWENKKNFSKQLRGEELSFKALSTKTGANAVDTDESSKPGLITNTVRGVADLLSPWRRKRRQVNPNTTNKCNPNLHYWTAQDEGAAVGLAWIPYFGPAAEGIYTEGIMHNQNGLICGLRQLANETTQALQLFLRATTELRTYSILNRKAIDFLLQRWGGTCRILGPDCCIEPHDWTKNITDKINQIIHDFIDNPLPDQDDDDNWWTGWRQWIPAGIGITGVIIAIIALLCICKFLC

SEQ ID NO: 7 - DNA, 2046 bp, synthetic construct
  misc_feature 1..2046: note = Marburgvirus ancestor (T2-11) Unoptimised
  source 1..2046: mol_type = other DNA
atgaagaccatatattttctgattagtctcattttaatccaaagtataaaaactctccctgttttagaaattgctagtaacagccaacctcaagatgtagattcagtgtgctccggaaccctccaaaagacagaagatgttcatctgatgggatttacactgagtgggcaaaaagttgctgattcccctttggaagcatctaaacgatgggctttcaggacaggtgttcctcccaagaacgttgagtatacggaaggagaagaagccaaaacatgttacaatataagtgtaacagacccttctggaaaatccttgctgctggatcctcccagtaatatccgcgattaccctaaatgtaaaactgttcatcatattcaaggtcaaaaccctcatgcacaggggattgccctccatttgtggggggcatttttcctgtatgatcgcattgcctccacaacaatgtaccgaggcaaagtcttcactgaagggaacatagcagctatgattgtcaataagacagtgcacaaaatgattttctcgaggcaaggacaagggtaccgtcacatgaatctgacttctactaataaatattggacaagtagcaacggaacgcaaacgaatgacactggatgcttcggtgctcttcaagaatacaattctacgaagaaccaaacatgtgctccgtccaaaatacctccaccactgcccacagcccgtccggagatcaaacccacaagcaccccaactgatgccaccaaactcaacaccacagacccaaacagtgatgatgaggacctcacaacatccggctcagggtccggagaacaggaaccctacacaacttctgatgcggtcactaagcaagggctttcatcaacaatgccacccactccctcaccacaaccaagcacgccacagcaaggaggaaacaacacaaaccattcccaaggtgctgtgactgaacccgacaaaaccaacacaactgcacaaccgtccatgcccccccacaacactactacaatctctactaacaacacctccaagcacaacttcagcactctctctgcaccactacaaaacaccaccaattacaacacacagagcacggccactgaaaatgagcaaaccagtgccccctcgaaaacaaccctgcctccaacaggaaatcctaccacagcaaagagcaccaacagcacaaaaggccccaccacaacggcaccaaatacgacaaatgggcatttcaccagtccctcccccacccccaactcgactacacaacatcttgtatatttcagaaggaaacgaagtatcctctggagggaaggcgacatgttcccttttttagatgggttaataaatactgaaattgattttgatccaatcccaaacacagaaacaatctttgatgaatcccccagctttaatacttcaactaatgaggaacaacacactcccccgaatatcagtttaactttctcttattttcctgataaaaatggagatactgcctactctggggaaaacgagaatgattgtgatgcagagttgaggatttggagtgtgcaggaggacgatttggcggcagggcttagctggataccattttttggccctggaatcgaaggactctatactgccggtttaatcaaaaatcagaacaatttagtttgtaggttgaggcgcttagctaatcaaactgctaaatccttggagctcttgttaagggtcacaaccgaggaaaggacattttccttaatcaataggcatgcaattgactttttgcttacgaggtggggcggaacatgcaaggtgctaggacctgattgttgcataggaatagaagatctatctaaaaatatctcagaacaaattgacaaaatcagaaaggatgaacaaaaggaggaaactggctggggtctaggtggcaaatggtggacatctgactggggtgttctcaccaatttgggcatcctgctactattatctatagctgttctgattgctctgtcctgtatctgtcgtatcttcactaaatatatcggatag

SEQ ID NO: 8 - DNA, 2046 bp, synthetic construct
  misc_feature 1..2046: note = Marburgvirus ancestor (T2-11) Gene optimised
  source 1..2046: mol_type = other DNA
atgaagaccatctactttctgatcagcctgatcctgatccagagcatcaagaccctgcctgtgctggaaatcgccagcaacagtcagccccaggatgtggatagcgtgtgtagcggcaccctccagaaaaccgaggatgtgcacctgatgggctttaccctgagcggccagaaagtggccgattctccactggaagccagcaagagatgggcctttagaaccggcgtgccacctaagaacgtcgagtacacagagggcgaagaggccaagacctgctacaacatcagcgtgaccgatcctagcggcaagagcctgctgctggaccctcctagcaacatcagagactaccccaagtgcaagaccgtgcaccacatccagggacagaatccccatgctcagggaattgccctgcacctgtggggcgcctttttcctgtatgatcggatcgcctccaccaccatgtacagaggcaaagtgttcaccgagggcaatatcgccgccatgatcgtgaacaagacagtgcacaagatgatcttcagccggcaaggccagggctacagacacatgaatctgaccagcaccaacaagtactggaccagcagcaacggcacccagaccaatgatacaggctgctttggcgccctgcaagagtacaacagcaccaagaatcagacatgcgcccctagcaagatccctcctccactgcctactgccagacctgagatcaagcctaccagcacacctaccgacgccaccaagctgaacaccaccgatccaaacagcgacgacgaggatctgacaacaagcggatctggctctggcgagcaagagccatacaccacctctgatgccgtgacaaagcagggcctgagcagcacaatgcctccaacaccttctccacagcctagcacacctcagcaaggcggcaacaacacaaatcactctcagggcgccgtgaccgagcctgacaagacaaataccacagctcagcccagcatgcctcctcacaacaccaccacaatctccaccaacaacaccagcaagcacaacttcagcacactgagcgcccctctccagaataccaccaactacaatacccagagcaccgccaccgagaacgagcagacatctgccccttctaagaccacactgccacctaccggcaatcctaccaccgccaagagcaccaatagcacaaagggccctaccaccaccgctcctaacaccacaaatggccacttcacaagcccaagtcctacacctaacagcacaacccagcacctggtgtacttcagacggaagcggagcatcctttggcgcgagggcgatatgttccctttcctggacggcctgatcaacaccgagatcgacttcgaccccattccaaacaccgaaaccatcttcgacgagagccccagcttcaacacctccaccaatgaggaacagcacacccctccaaacatctccctgaccttcagctacttccccgacaagaacggcgatacagcctacagcggcgagaatgagaatgactgcgacgccgagctgcggatttggagcgttcaagaggatgatctggctgccggcctgagctggatccctttttttggacctggcatcgagggcctgtacaccgccggactgatcaagaaccagaacaacctcgtgtgcagactgcggagactggccaatcagaccgccaagtctctggaactgctgctgcgcgtgaccaccgaggaaagaaccttctctctgatcaaccggcacgccatcgattttctgctgaccagatggggcggcacctgtaaagttctgggccctgattgctgcatcggaatcgaggacctgagcaagaacatctccgagcagatcgacaagatccgcaaggacgagcagaaagaggaaacaggctggggactcggcggcaagtggtggacatctgattggggcgtgctgaccaatctgggaatcctgctgctcctgtctatcgccgtgctgatcgccctgagctgcatctgccggatcttcaccaagtacatcggctga

SEQ ID NO: 9 - protein, 681 aa, synthetic construct
  REGION 1..681: note = Marburgvirus ancestor (T2-11)
MKTIYFLISLILIQSIKTLPVLEIASNSQPQDVDSVCSGTLQKTEDVHLMGFTLSGQKVADSPLEASKRWAFRTGVPPKNVEYTEGEEAKTCYNISVTDPSGKSLLLDPPSNIRDYPKCKTVHHIQGQNPHAQGIALHLWGAFFLYDRIASTTMYRGKVFTEGNIAAMIVNKTVHKMIFSRQGQGYRHMNLTSTNKYWTSSNGTQTNDTGCFGALQEYNSTKNQTCAPSKIPPPLPTARPEIKPTSTPTDATKLNTTDPNSDDEDLTTSGSGSGEQEPYTTSDAVTKQGLSSTMPPTPSPQPSTPQQGGNNTNHSQGAVTEPDKTNTTAQPSMPPHNTTTISTNNTSKHNFSTLSAPLQNTTNYNTQSTATENEQTSAPSKTTLPPTGNPTTAKSTNSTKGPTTTAPNTTNGHFTSPSPTPNSTTQHLVYFRRKRSILWREGDMFPFLDGLINTEIDFDPIPNTETIFDESPSFNTSTNEEQHTPPNISLTFSYFPDKNGDTAYSGENENDCDAELRIWSVQEDDLAAGLSWIPFFGPGIEGLYTAGLIKNQNNLVCRLRRLANQTAKSLELLLRVTTEERTFSLINRHAIDFLLTRWGGTCKVLGPDCCIGIEDLSKNISEQIDKIRKDEQKEETGWGLGGKWWTSDWGVLTNLGILLLLSIAVLIALSCICRIFTKYIG

SEQ ID NO: 10 - DNA, 1578 bp, synthetic construct; identical to SEQ ID NO: 2
  misc_feature 1..1578: note = Tier 2-4 (SUDV_anc_-MLD)
  source 1..1578: mol_type = other DNA

SEQ ID NO: 11 - protein, 525 aa, synthetic construct; identical to SEQ ID NO: 3
  REGION 1..525: note = Tier 2-4 (SUDV_anc_-MLD)

SEQ ID NO: 12 - DNA, 1581 bp, synthetic construct; identical to SEQ ID NO: 5
  misc_feature 1..1581: note = Tier 2-6 (SUDV_EBOV-TAFV-BDBV_anc_-MLD)
  source 1..1581: mol_type = other DNA

SEQ ID NO: 13 - protein, 526 aa, synthetic construct; identical to SEQ ID NO: 6
  REGION 1..526: note = Tier 2-6 (SUDV_EBOV-TAFV-BDBV_anc_-MLD)

SEQ ID NO: 14 - DNA, 2046 bp, synthetic construct; identical to SEQ ID NO: 8
  misc_feature 1..2046: note = Tier 2-11 (RAVV_MARV_anc)
  source 1..2046: mol_type = other DNA

SEQ ID NO: 15 - protein, 681 aa, synthetic construct; identical to SEQ ID NO: 9
  REGION 1..681: note = Tier 2-11 (RAVV_MARV_anc)

SEQ ID NO: 16 - DNA, 91 bp, synthetic construct
  misc_feature 1..91: note = pEVAC Multiple Cloning Site
  source 1..91: mol_type = other DNA
acagactgttcctttccatgggtcttttctgcagtcaccgtcggtaccgtcgacacgtgtgatcatctagaggatccgcggccgcagatct

SEQ ID NO: 17 - DNA, 4405 bp, synthetic construct
  misc_feature 1..4405: note = Entire Sequence of pEVAC
  source 1..4405: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcggtaccgtcgacacgtgtgatcatctagaggatccgcggccgcagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 18 - protein, 491 aa, synthetic construct
  REGION 1..491: note = L-10 = LASV_III_IV_anc
MGQIVTFFQEVPHVIEEVMNIVLIALSLLAILKGLYNVATCGLIGLVTFLLLCGRSCSTTLYKGVYELQTLELNMETLNMTMPLSCTKNNSHHYIRVGNETGLELTLTNTSIINHKFCNLSDAHKKNLYDHALMSIISTFHLSIPNFNQYEAMSCDFNGGKISVQYNLSHSYAVDAANHCGTVANGVLQTFMRMAWGGSYIALDSGRGNWDCIMTSYQYLIIQNTTWEDHCQFSRPSPIGYLGLLSQRTRDIYISRRLLGTFTWTLSDSEGNETPGGYCLTRWMLIEAELKCFGNTAVAKCNEKHDEEFCDMLRLFDFNKQAIRRLKAEAQMSIQLINKAVNALINDQLIMKNHLRDIMGIPYCNYSKYWYLNHTITGKTSLPKCWLVSNGSYLNETHFSDDIEQQADNMITEMLQKEYMDRQGKTPLGLVDLFVFSTSFYLISIFLHLVKIPTHRHIVGKPCPKPHRLNHMGICSCGLYKQPGVPVRWKR

SEQ ID NO: 19 - DNA, 1476 bp, synthetic construct
  misc_feature 1..1476: note = L-10 = LASV_III_IV_anc
  source 1..1476: mol_type = other DNA
atgggccagatcgtgacattcttccaagaggtgccccacgtgatcgaagaagtgatgaacatcgtcctgatcgccctgagcctgctggccatcctgaagggcctgtataatgtggccacctgtggcctgatcggcctggtcacatttctgctgctgtgcggcagaagctgctccaccacactgtataagggcgtgtacgagctgcaaaccctggaactgaacatggaaaccctgaacatgaccatgcctctgagctgcaccaagaacaacagccaccactacatcagagtgggcaacgagacaggcctcgagctgaccctgaccaacaccagcatcatcaaccacaagttctgcaacctgagcgacgcccacaagaagaacctgtacgatcacgccctgatgagcatcatctccaccttccacctgagcatccccaacttcaaccagtacgaggccatgagctgcgacttcaacggcggaaagatcagcgtgcagtacaatctgagccacagctatgccgtggacgccgccaatcattgtggaacagtggccaatggcgtgctccagaccttcatgagaatggcctggggcggcagctatatcgccctggattctggcagaggcaactgggactgcatcatgaccagctaccagtacctgatcatccagaacaccacctgggaagatcactgccagttcagcagaccctctcctatcggatacctgggcctgctgtcccagagaacccgggacatctacatctctagacggctgctgggcaccttcacctggacactgtctgatagcgagggcaatgagacacctggcggctactgtctgacccggtggatgctgattgaggccgagctgaagtgcttcggaaataccgccgtggccaagtgcaacgagaagcacgacgaggaattctgcgacatgctgcggctgttcgatttcaacaagcaggccatcagacggctgaaggccgaggctcagatgtccatccagctgatcaacaaggccgtgaatgccctgattaacgaccagctcatcatgaagaaccacctcagggacatcatgggcatcccttactgcaactacagcaagtactggtatctgaaccacaccatcaccggcaagaccagcctgcctaagtgctggctggtgtccaacggcagctacctgaacgagacacacttcagcgacgacatcgagcagcaggccgacaacatgatcaccgagatgctccagaaagagtacatggaccggcagggcaagacacctctgggccttgtggatctgttcgtgttcagcaccagcttctacctgatctctatcttcctgcacctggtcaagatccccacacacagacacatcgtgggcaagccctgtcctaagcctcacagactgaaccatatgggcatctgtagctgcggcctgtacaaacagcctggcgtgccagtgcggtggaagagataa

SEQ ID NO: 20 - protein, 491 aa, synthetic construct
  REGION 1..491: note = L-10-SOSEP
MGQIVTFFQEVPHVIEEVMNIVLIALSLLAILKGLYNVATCGLIGLVTFLLLCGRSCSTTLYKGVYELQTLELNMETLNMTMPLSCTKNNSHHYIRVGNETGLELTLTNTSIINHKFCNLSDAHKKNLYDHALMSIISTFHLSIPNFNQYEAMSCDFNGGKISVQYNLSHSYAVDAANHCGTVANGVLQTFMRMAWGGSYIALDSGCGNWDCIMTSYQYLIIQNTTWEDHCQFSRPSPIGYLGLLSQRTRDIYISRRRRGTFTWTLSDSEGNETPGGYCLTRWMLIEAELKCFGNTAVAKCNEKHDEEFCDMLRLFDFNKQAIRRLKAPAQMSIQLINKAVNALINDQLIMKNHLRDIMCIPYCNYSKYWYLNHTITGKTSLPKCWLVSNGSYLNETHFSDDIEQQADNMITEMLQKEYMDRQGKTPLGLVDLFVFSTSFYLISIFLHLVKIPTHRHIVGKPCPKPHRLNHMGICSCGLYKQPGVPVRWKR

SEQ ID NO: 21 - DNA, 1476 bp, synthetic construct
  misc_feature 1..1476: note = L-10-SOSEP
  source 1..1476: mol_type = other DNA
atgggccagatcgtgacattcttccaagaggtgccccacgtgatcgaagaagtgatgaacatcgtcctgatcgccctgagcctgctggccatcctgaagggcctgtataatgtggccacctgtggcctgatcggcctggtcacatttctgctgctgtgcggcagaagctgctccaccacactgtataagggcgtgtacgagctgcaaaccctggaactgaacatggaaaccctgaacatgaccatgcctctgagctgcaccaagaacaacagccaccactacatcagagtgggcaacgagacaggcctcgagctgaccctgaccaacaccagcatcatcaaccacaagttctgcaacctgagcgacgcccacaagaagaacctgtacgatcacgccctgatgagcatcatctccaccttccacctgagcatccccaacttcaaccagtacgaggccatgagctgcgacttcaacggcggaaagatcagcgtgcagtacaatctgagccacagctatgccgtggacgccgccaatcattgtggaacagtggccaatggcgtgctccagaccttcatgagaatggcctggggcggcagctatatcgccctggattctggctgtggcaactgggactgcatcatgaccagctaccagtacctgatcatccagaacaccacctgggaagatcactgccagttcagcagaccctctcctatcggatacctgggcctgctgtcccagagaacccgggacatctacatctctcggcggagaagaggcaccttcacctggacactgtctgatagcgagggcaatgagacacctggcggctactgtctgacccggtggatgctgattgaggccgagctgaagtgcttcggaaataccgccgtggccaagtgcaacgagaagcacgacgaggaattctgcgacatgctgcggctgttcgatttcaacaagcaggccatcagacggctgaaggcccctgctcagatgtccatccagctgatcaacaaggccgtgaatgccctgattaacgaccagctcatcatgaagaaccacctcagggacatcatgtgcatcccttactgcaactacagcaagtactggtatctgaaccacaccatcaccggcaagaccagcctgcctaagtgctggctggtgtccaacggcagctacctgaacgagacacacttcagcgacgacatcgagcagcaggccgacaacatgatcaccgagatgctccagaaagagtacatggaccggcagggcaagacacctctgggccttgtggatctgttcgtgttcagcaccagcttctacctgatctctatcttcctgcacctggtcaagatccccacacacagacacatcgtgggcaagccctgtcctaagcctcacagactgaaccatatgggcatctgtagctgcggcctgtacaaacagcctggcgtgccagtgcggtggaagagataa

SEQ ID NO: 22 - protein, 491 aa, synthetic construct
  REGION 1..491: note = L-10-SOSEP-NtoK
MGQIVTFFQEVPHVIEEVMNIVLIALSLLAILKGLYNVATCGLIGLVTFLLLCGRSCSTTLYKGVYELQTLELNMETLNMTMPLSCTKNNSHHYIRVGNETGLELTLTNTSIINHKFCNLSDAHKKNLYDHALMSIISTFHLSIPNFNQYEAMSCDFNGGKISVQYNLSHSYAVDAANHCGTVANGVLQTFMRMAWGGSYIALDSGCGNWDCIMTSYQYLIIQNTTWEDHCQFSRPSPIGYLGLLSQRTRDIYISRRRRGTFTWTLSDSEGKETPGGYCLTRWMLIEAELKCFGNTAVAKCNEKHDEEFCDMLRLFDFNKQAIRRLKAPAQMSIQLINKAVNALINDQLIMKNHLRDIMCIPYCNYSKYWYLNHTITGKTSLPKCWLVSNGSYLNETHFSDDIEQQADNMITEMLQKEYMDRQGKTPLGLVDLFVFSTSFYLISIFLHLVKIPTHRHIVGKPCPKPHRLNHMGICSCGLYKQPGVPVRWKR

SEQ ID NO: 23 - DNA, 1476 bp, synthetic construct
  misc_feature 1..1476: note = L-10-SOSEP-NtoK
  source 1..1476: mol_type = other DNA
atgggccagatcgtgacattcttccaagaggtgccccacgtgatcgaagaagtgatgaacatcgtcctgatcgccctgagcctgctggccatcctgaagggcctgtataatgtggccacctgtggcctgatcggcctggtcacatttctgctgctgtgcggcagaagctgctccaccacactgtataagggcgtgtacgagctgcaaaccctggaactgaacatggaaaccctgaacatgaccatgcctctgagctgcaccaagaacaacagccaccactacatcagagtgggcaacgagacaggcctcgagctgaccctgaccaacaccagcatcatcaaccacaagttctgcaacctgagcgacgcccacaagaagaacctgtacgatcacgccctgatgagcatcatctccaccttccacctgagcatccccaacttcaaccagtacgaggccatgagctgcgacttcaacggcggaaagatcagcgtgcagtacaatctgagccacagctatgccgtggacgccgccaatcattgtggaacagtggccaatggcgtgctccagaccttcatgagaatggcctggggcggcagctatatcgccctggattctggctgtggcaactgggactgcatcatgaccagctaccagtacctgatcatccagaacaccacctgggaagatcactgccagttcagcagaccctctcctatcggatacctgggcctgctgtcccagagaacccgggacatctacatctctcggcggagaagaggcaccttcacctggacactgtctgatagcgagggcaaagagacacctggcggctactgtctgacccggtggatgctgattgaggccgagctgaagtgcttcggaaataccgccgtggccaagtgcaacgagaagcacgacgaggaattctgcgacatgctgcggctgttcgatttcaacaagcaggccatcagacggctgaaggcccctgctcagatgtccatccagctgatcaacaaggccgtgaatgccctgattaacgaccagctcatcatgaagaaccacctcagggacatcatgtgcatcccttactgcaactacagcaagtactggtatctgaaccacaccatcaccggcaagaccagcctgcctaagtgctggctggtgtccaacggcagctacctgaacgagacacacttcagcgacgacatcgagcagcaggccgacaacatgatcaccgagatgctccagaaagagtacatggaccggcagggcaagacacctctgggccttgtggatctgttcgtgttcagcaccagcttctacctgatctctatcttcctgcacctggtcaagatccccacacacagacacatcgtgggcaagccctgtcctaagcctcacagactgaaccatatgggcatctgtagctgcggcctgtacaaacagcctggcgtgccagtgcggtggaagagataa

SEQ ID NO: 24 - protein, 497 aa, synthetic construct
  REGION 1..497: note = L-10-FLEP
MGQIVTFFQEVPHVIEEVMNIVLIALSLLAILKGLYNVATCGLIGLVTFLLLCGRSCSTTLYKGVYELQTLELNMETLNMTMPLSCTKNNSHHYIRVGNETGLELTLTNTSIINHKFCNLSDAHKKNLYDHALMSIISTFHLSIPNFNQYEAMSCDFNGGKISVQYNLSHSYAVDAANHCGTVANGVLQTFMRMAWGGSYIALDSGRGNWDCIMTSYQYLIIQNTTWEDHCQFSRPSPIGYLGLLSQRTRDIYISGGGGSGGGGSGTFTWTLSDSEGNETPGGYCLTRWMLIEAELKCFGNTAVAKCNEKHDEEFCDMLRLFDFNKQAIRRLKAPAQMSIQLINKAVNALINDQLIMKNHLRDIMGIPYCNYSKYWYLNHTITGKTSLPKCWLVSNGSYLNETHFSDDIEQQADNMITEMLQKEYMDRQGKTPLGLVDLFVFSTSFYLISIFLHLVKIPTHRHIVGKPCPKPHRLNHMGICSCGLYKQPGVPVRWKR

SEQ ID NO: 25 - DNA, 1494 bp, synthetic construct
  misc_feature 1..1494: note = L-10-FLEP
  source 1..1494: mol_type = other DNA
atgggccagatcgtgacattcttccaagaggtgccccacgtgatcgaagaagtgatgaacatcgtcctgatcgccctgagcctgctggccatcctgaagggcctgtataatgtggccacctgtggcctgatcggcctggtcacatttctgctgctgtgcggcagaagctgctccaccacactgtataagggcgtgtacgagctgcaaaccctggaactgaacatggaaaccctgaacatgaccatgcctctgagctgcaccaagaacaacagccaccactacatcagagtgggcaacgagacaggcctcgagctgaccctgaccaacaccagcatcatcaaccacaagttctgcaacctgagcgacgcccacaagaagaacctgtacgatcacgccctgatgagcatcatctccaccttccacctgagcatccccaacttcaaccagtacgaggccatgagctgcgacttcaacggcggaaagatcagcgtgcagtacaatctgagccacagctatgccgtggacgccgccaatcattgtggaacagtggccaatggcgtgctccagaccttcatgagaatggcctggggcggcagctatatcgccctggattctggcagaggcaactgggactgcatcatgaccagctaccagtacctgatcatccagaacaccacctgggaagatcactgccagttcagcagaccctctcctatcggatacctgggcctgctgtcccagagaacccgggacatctacatctctggcggcggaggatctggcggaggtggaagtggcaccttcacctggacactgtctgatagcgagggcaatgagacacctggcggctactgtctgacccggtggatgctgattgaggccgagctgaagtgcttcggaaataccgccgtggccaagtgcaacgagaagcacgacgaggaattctgcgacatgctgcggctgttcgatttcaacaagcaggccatcagacggctgaaggcccctgctcagatgtccatccagctgatcaacaaggccgtgaatgccctgattaacgaccagctcatcatgaagaaccacctcagggacatcatgggcatcccttactgcaactacagcaagtactggtatctgaaccacaccatcaccggcaagaccagcctgcctaagtgctggctggtgtccaacggcagctacctgaacgagacacacttcagcgacgacatcgagcagcaggccgacaacatgatcaccgagatgctccagaaagagtacatggaccggcagggcaagacacctctgggccttgtggatctgttcgtgttcagcaccagcttctacctgatctctatcttcctgcacctggtcaagatccccacacacagacacatcgtgggcaagccctgtcctaagcctcacagactgaaccatatgggcatctgtagctgcggcctgtacaaacagcctggcgtgccagtgcggtggaagagataa

SEQ ID NO: 26 - protein, 497 aa, synthetic construct
  REGION 1..497: note = L-10-FLEP-NtoK
MGQIVTFFQEVPHVIEEVMNIVLIALSLLAILKGLYNVATCGLIGLVTFLLLCGRSCSTTLYKGVYELQTLELNMETLNMTMPLSCTKNNSHHYIRVGNETGLELTLTNTSIINHKFCNLSDAHKKNLYDHALMSIISTFHLSIPNFNQYEAMSCDFNGGKISVQYNLSHSYAVDAANHCGTVANGVLQTFMRMAWGGSYIALDSGRGNWDCIMTSYQYLIIQNTTWEDHCQFSRPSPIGYLGLLSQRTRDIYISGGGGSGGGGSGTFTWTLSDSEGKETPGGYCLTRWMLIEAELKCFGNTAVAKCNEKHDEEFCDMLRLFDFNKQAIRRLKAPAQMSIQLINKAVNALINDQLIMKNHLRDIMGIPYCNYSKYWYLNHTITGKTSLPKCWLVSNGSYLNETHFSDDIEQQADNMITEMLQKEYMDRQGKTPLGLVDLFVFSTSFYLISIFLHLVKIPTHRHIVGKPCPKPHRLNHMGICSCGLYKQPGVPVRWKR

SEQ ID NO: 27 - DNA, 1494 bp, synthetic construct
  misc_feature 1..1494: note = L-10-FLEP-NtoK
  source 1..1494: mol_type = other DNA
atgggccagatcgtgacattcttccaagaggtgccccacgtgatcgaagaagtgatgaacatcgtcctgatcgccctgagcctgctggccatcctgaagggcctgtataatgtggccacctgtggcctgatcggcctggtcacatttctgctgctgtgcggcagaagctgctccaccacactgtataagggcgtgtacgagctgcaaaccctggaactgaacatggaaaccctgaacatgaccatgcctctgagctgcaccaagaacaacagccaccactacatcagagtgggcaacgagacaggcctcgagctgaccctgaccaacaccagcatcatcaaccacaagttctgcaacctgagcgacgcccacaagaagaacctgtacgatcacgccctgatgagcatcatctccaccttccacctgagcatccccaacttcaaccagtacgaggccatgagctgcgacttcaacggcggaaagatcagcgtgcagtacaatctgagccacagctatgccgtggacgccgccaatcattgtggaacagtggccaatggcgtgctccagaccttcatgagaatggcctggggcggcagctatatcgccctggattctggcagaggcaactgggactgcatcatgaccagctaccagtacctgatcatccagaacaccacctgggaagatcactgccagttcagcagaccctctcctatcggatacctgggcctgctgtcccagagaacccgggacatctacatctctggcggcggaggatctggcggaggtggaagtggcaccttcacctggacactgtctgatagcgagggcaaagagacacctggcggctactgtctgacccggtggatgctgattgaggccgagctgaagtgcttcggaaataccgccgtggccaagtgcaacgagaagcacgacgaggaattctgcgacatgctgcggctgttcgatttcaacaagcaggccatcagacggctgaaggcccctgctcagatgtccatccagctgatcaacaaggccgtgaatgccctgattaacgaccagctcatcatgaagaaccacctcagggacatcatgggcatcccttactgcaactacagcaagtactggtatctgaaccacaccatcaccggcaagaccagcctgcctaagtgctggctggtgtccaacggcagctacctgaacgagacacacttcagcgacgacatcgagcagcaggccgacaacatgatcaccgagatgctccagaaagagtacatggaccggcagggcaagacacctctgggccttgtggatctgttcgtgttcagcaccagcttctacctgatctctatcttcctgcacctggtcaagatccccacacacagacacatcgtgggcaagccctgtcctaagcctcacagactgaaccatatgggcatctgtagctgcggcctgtacaaacagcctggcgtgccagtgcggtggaagagataa

SEQ ID NO: 28 - protein, 569 aa, synthetic construct
  REGION 1..569: note = L-NP-1 = L-NP-CovAnc-1_N
MSASKEVKSFLWTQSLRRELSGYCSNIKLQVVKDAQALLHGLDFSEVSNVQRLMRKQKRDDSDLKRLRDLNQAVNNLVELKSTQQKSILRVGTLTSDDLLTLAADLEKLKSKVIRTERPLSSGVYMGNLSTQQLEQRRALLNMIGMVGGAQGTQPGRDGVVRVWDVKNPDLLNNQFGTMPSLTLACLTKQGQVDLNDAVLALTDLGLIYTAKYPNSSDLDRLSQSHPILNMVDTKKSSLNISGYNFSLGAAVKAGACMLDGGNMLETIKVTPQTMDGILKSILKVKKSLGMFVSDTPGERNPYENILYKICLSGDGWPYIASRTSIVGRAWENTTVDLESDGKPQKVGTAGSNKSLQSAGFPTGLTYSQLMTLKDSMMQLDPSAKTWIDIEGRPEDPVEIALYQPMSGCYIHFFREPTDLKQFKQDAKYSHGIDVADLFPAQPGLTSAVIEALPRNMVLTCQGSDDIKRLLDSQGRRDIKLIDIALSKADSRRFENAVWDQCKDLCHMHTGVVVEKKKRGGKEEITPHCALMDCIMYDAAVSGGLNIPVLRAVLPRDMVFRTSSPKVVL

SEQ ID NO: 29 - DNA, 1710 bp, synthetic construct
  misc_feature 1..1710: note = L-NP-1 = L-NP-CovAnc-1_N
  source 1..1710: mol_type = other DNA
atgagcgccagcaaagaagtgaaaagcttcctctggacccagagcctgcggagagagctgtctggctactgctccaacatcaagctccaggtggtcaaggacgcccaggctctgctgcatggcctggatttcagcgaggtgtccaacgtgcagcggctgatgagaaagcagaagcgggacgacagcgacctgaagagactgagggatctgaaccaggccgtgaacaacctggtggaactgaagtctacccagcagaaatccatcctgagagtgggcaccctgaccagcgacgatctgctgacactggccgccgatctggaaaagctgaagtccaaagtgatccggaccgagaggccactgtctagcggagtgtacatgggcaacctgagcacccagcagctggaacagagaagggccctgctgaacatgatcggcatggttggaggcgcccagggaacacagcctggaagagatggtgtcgtcagagtgtgggacgtgaagaaccccgacctgctcaacaaccagttcggcaccatgccttctctgaccctggcctgcctgacaaagcagggccaagtggacctgaacgatgccgtgctggctctgactgatctgggcctgatctacaccgccaagtatcccaacagctccgacctggacaggctgagccagtctcaccccatcctgaacatggtggacaccaagaagtccagcctgaacatcagcggctacaacttctctctgggcgctgccgtgaaagccggcgcttgtatgcttgacggcggcaacatgctggaaaccatcaaagtgacccctcagaccatggacggcatcctgaaaagtatcctgaaagtgaagaaatccctgggcatgttcgtgtccgacacacccggcgagagaaacccctacgagaacatcctgtacaagatttgcctgagcggcgacggctggccctatatcgccagcagaacatctatcgtgggcagagcttgggagaacaccaccgtggacctggaatccgatggcaagcctcagaaagtgggcacagccggcagcaacaagagcctccagtctgccggatttcctaccggcctgacatacagccagctgatgaccctgaaggacagcatgatgcagctggaccctagcgccaagacctggatcgacattgagggcagacccgaggatcccgtggaaatcgctctgtaccagcctatgagcggctgctatatccacttcttcagagagcccaccgatctgaagcagttcaagcaggacgccaagtacagccacggaatcgacgtggccgatctgttcccagctcagccaggactgacatccgccgtgattgaagccctgcctagaaacatggtgctgacctgtcagggcagcgacgacatcaagagactgctggacagccagggcagaagagatatcaagctgatcgatatcgccctgagcaaggccgactctcggagattcgaaaacgccgtgtgggaccagtgcaaggacctgtgtcacatgcacacaggcgtggtggtggaaaagaagaagcgcggaggcaaagaggaaatcacccctcactgcgccctgatggactgcattatgtatgacgccgccgtgtctggcggcctgaatatccctgttctgagagccgtgctgccccgcgacatggtgtttagaacaagcagccccaaggtggtgctctga

SEQ ID NO: 30 - protein, 569 aa, synthetic construct
  REGION 1..569: note = L-NP-1 = L-NP-CovAnc-2_SL
MSASKEIKSFLWTQSLRRELSGYCSNIKLQVVKDAQALLHGLDFSEVSNVQRLMRKERRDDNDLKRLRDLNQAVNNLVELKSTQQKSILRVGTLTSDDLLILAADLEKLKSKVTRTERPLSAGVYMGNLSSQQLDQRRALLNMIGMSGGNQGARAGRDGVVRVWDVKNAELLNNQFGTMPSLTLACLTKQGQVDLNDAVQALTDLGLIYTAKYPNTSDLDRLTQSHPILNMIDTKKSSLNISGYNFSLGAAVKAGACMLDGGNMLETIKVSPQTMDGILKSILKVKKALGMFISDTPGERNPYENILYKICLSGDGWPYIASRTSITGRAWENTVVDLESDGKPQKAGSNNSNKSLQSAGFTAGLTYSQLMTLKDAMLQLDPNAKTWMDIEGRPEDPVEIALYQPSSGCYIHFFREPTDLKQFKQDAKYSHGIDVTDLFAAQPGLTSAVIDALPRNMVITCQGSDDIRKLLESQGRKDIKLIDIALSKTDSRKYENAVWDQYKDLCHMHTGVVVEKKKRGGKEEITPHCALMDCIMFDAAVSGGLNTSVLRAVLPRDMVFRTSTPRVVL

SEQ ID NO: 31 - DNA, 1710 bp, synthetic construct
  misc_feature 1..1710: note = L-NP-1 = L-NP-CovAnc-2_SL
  source 1..1710: mol_type = other DNA
atgagcgccagcaaagagatcaagagcttcctgtggacccagagcctgcggagagagctgtctggctactgctccaacatcaagctccaggtggtcaaggacgcccaggctctgctgcatggcctggatttcagcgaggtgtccaacgtgcagcggctgatgcggaaagagagaagggacgacaacgacctgaagcggctgagggatctgaaccaggccgtgaacaacctggtggaactgaagtctacccagcagaaatccatcctgagagtgggcaccctgaccagcgacgatctgctgattctggccgccgacctggaaaagctgaagtccaaagtgacccggaccgagaggccactgtctgctggtgtctacatgggcaacctgagcagccagcagctggatcagagaagggccctgctgaacatgatcggcatgagcggcggaaatcagggcgctagagctggcagagatggcgtcgtcagagtgtgggacgtgaagaatgccgagctgctcaacaaccagttcggcaccatgcctagcctgacactggcctgcctgacaaagcagggccaagtggacctgaacgatgctgtgcaggccctgactgatctgggcctgatctacaccgccaagtatcccaacaccagcgacctggacagactgacccagtctcaccccatcctgaatatgatcgacaccaagaagtccagcctgaacatcagcggctacaacttctctctgggcgctgccgtgaaagccggcgcttgtatgcttgacggcggcaacatgctggaaaccatcaaggtgtccccacagaccatggacggcatcctgaaaagtatcctgaaagtgaagaaagccctgggcatgttcatcagcgacacccctggcgagagaaacccctacgagaacatcctgtacaagatttgcctgagcggcgacggctggccctatatcgccagcagaaccagcattaccggcagagcttgggagaacaccgtggtggatctggaaagcgacggcaagcctcagaaggccggcagcaacaactccaacaagagcctccagtccgccggcttcacagccggcctgacatatagccagctgatgaccctgaaggacgccatgctgcaactggaccccaatgccaagacctggatggacatcgagggcagacctgaggaccctgtggaaatcgccctgtaccagcctagctccggctgctatatccacttcttcagagagcccaccgatctgaagcagttcaagcaggacgccaagtacagccacggcatcgacgtgaccgatctgtttgctgctcagcccggactgacctccgccgtgattgatgccctgcctcggaacatggtcatcacctgtcagggcagcgacgacatccggaagctgctggaatctcagggcagaaaggatatcaagctgatcgatatcgccctgagcaagaccgacagccggaagtacgaaaacgccgtgtgggaccagtacaaggacctgtgccacatgcacacaggcgtggtggtggaaaagaagaagcgcggaggcaaagaggaaatcacccctcactgcgctctgatggactgcatcatgtttgacgccgccgtgtctggcggcctgaatacctctgttctgagagccgtgctgcccagagacatggtgttcagaacaagcacccctagagtggtgctctga

(19) United States
(12) Patent Application Publication
(10) Pub. No.: US 2026/0224686 A1
(43) Pub. Date: Aug. 6, 2026

(54) VACCINES AND METHODS

(71) Applicants: CAMBRIDGE ENTERPRISE LIMITED, Cambridge (GB); UNIVERSITY OF WESTMINSTER, London (GB); UNIVERSITAT REGENSBURG, Regensburg (DE)

(72) Inventors: Jonathan Luke HEENEY, Cambridge, Cambridgeshire (GB); Simon FROST, Cambridge, Cambridgeshire (GB); Ralf WAGNER, Regensburg (DE); Benedikt ASBACH, Regensburg (DE); Rebecca KINSLEY, Cambridge, Cambridgeshire (GB); Edward WRIGHT, London, Greater London (GB)

(73) Assignees: CAMBRIDGE ENTERPRISE LIMITED, Cambridge (GB); UNIVERSITY OF WESTMINSTER, London (GB); UNIVERSITAT REGENSBURG, Regensburg (DE)

(21) Appl. No.: 19/437,409

(22) Filed: Dec. 31, 2025

Related U.S. Application Data
(63) Continuation of application No. 17/280,526, filed on Sep. 27, 2019, now abandoned.

(30) Foreign Application Priority Data
Sep. 28, 2018 (GB) 1815956.6

(51) Int. Cl. A61K 39/12; C07K 16/10; C07K 16/108; C12N 7/00

(52) U.S. Cl. CPC A61K 39/12; C07K 16/10; C07K 16/108; C12N 7/00; C12N 2760/10022; C12N 2760/10034; C12N 2760/14022; C12N 2760/14034; C12N 2760/14122; C12N 2760/14134; C12N 2760/16122; C12N 2760/16134

(57) ABSTRACT

Methods for identifying optimized antigenic pathogen polypeptides capable of inducing a broadly neutralizing immune response, and associated T-cell responses, to a pathogen are described, as well as nucleic acid sequences encoding such polypeptides. Methods for determining whether a broadly neutralizing immune response is induced in a subject following immunization with an optimized antigenic pathogen polypeptide, or a nucleic acid encoding the optimized pathogen polypeptide, are also described. Nucleic acid molecules, polypeptides, vectors, cells, fusion proteins, pharmaceutical compositions, and their use as vaccines against pathogens, especially against emerging or re-emerging pathogens (particularly RNA viruses), are also described.

### CROSS REFERENCE TO RELATED APPLICATIONS

This application is a continuation of U.S. patent application Ser. No. 17/280,526, filed Mar. 26, 2021, which is a National Stage Entry of International Application No. PCT/GB2019/052747, filed Sep. 27, 2019, which claims priority from GB Application No. 1815956.6, filed Sep. 28, 2018, the entire contents of all of which are incorporated by reference herein.

### INCORPORATION BY REFERENCE OF SEQUENCE LISTING

The Sequence Listing in the XML file, named as 39433Z_SequenceListing.xml of 77,824 bytes, created on Dec. 24, 2025 and submitted to the United States Patent and Trademark Office via Patent Center, is incorporated herein by reference.

This invention relates to methods for identifying optimized antigenic pathogen polypeptides capable of inducing a broadly neutralizing immune response to a pathogen, to methods for identifying a nucleic acid sequence encoding such optimized antigenic pathogen polypeptides, and to methods for determining whether a broadly neutralizing immune response is induced in a subject following immunization with an optimized antigenic pathogen polypeptide or a nucleic acid encoding the optimized pathogen polypeptide. The invention also relates to nucleic acid molecules, polypeptides, vectors, cells, fusion proteins, pharmaceutical compositions, and their use as vaccines against pathogens, especially against emerging or re-emerging pathogens (particularly RNA viruses). The invention also relates to pseudotyped virus particles.

The fundamental principal of a vaccine is to prepare the immune system for an encounter with a pathogen. A vaccine triggers the immune system to produce antibodies and T-cell responses, which help to combat infection. Historically, once a pathogen was isolated and grown, it was either mass produced and killed or attenuated, and used as a vaccine. Later recombinant genes from isolated pathogens were used to generate recombinant proteins that were mixed with adjuvants to stimulate immune responses. More recently the pathogen genes were cloned into vector systems (attenuated bacteria or viruses) to express and deliver the antigen in vivo. All of these strategies are dependent on pathogens isolated from past outbreaks to prevent future ones. For pathogens which do not change significantly, or slowly, this conventional technology is effective. However, some pathogens, are prone to mutating and antibodies do not always recognise different strains of the pathogen. New emerging and re-emerging pathogens often hide or disguise their vulnerable antigens from the immune system.

Of the emerging and re-emerging diseases, a disproportionate number (37%) are caused by ribonucleic acid (RNA) viruses (Heeney, Journal of Internal Medicine 2006; 260: 399-408). An RNA virus is a virus that has RNA as its genetic material. This nucleic acid is usually single-stranded RNA (ssRNA) but may be double-stranded RNA (dsRNA). RNA viruses generally have very high mutation rates compared to DNA viruses, because viral RNA polymerases lack the proofreading ability of DNA polymerases. This is one reason why it is difficult to make effective vaccines to prevent diseases caused by RNA viruses. In most cases, current vaccine candidates against RNA viruses are limited by the viral strain used as the vaccine insert, which is often chosen based on availability of a wild-type strain rather than by informed design. Technical challenges for developing vaccines for enveloped RNA viruses include: i) viral variation of wild-type field isolate glycoproteins (GPs) provide limited breadth of protection as vaccine antigens; ii) selection of vaccine antigens expressed by the vaccine inserts is highly empirical; immunogen selection is a slow, trial and error process; iii) in an evolving or unanticipated viral epidemic, developing new vaccine candidates is time-consuming and can delay vaccine deployment.

Notable human diseases caused by RNA viruses include viral hemorrhagic fevers (VHFs), a group of illnesses that are caused by several distinct families of viruses. In general, the term “viral hemorrhagic fever” is used to describe a severe multisystem syndrome (i.e. multiple organ systems in the body are affected). Characteristically, the overall vascular system is damaged, and the body's ability to regulate itself is impaired. These symptoms are often accompanied by hemorrhage (bleeding), although the bleeding is itself rarely life-threatening. While some types of hemorrhagic fever viruses can cause relatively mild illnesses, many of the viruses cause severe, life-threatening disease. VHFs are caused by viruses of at least five distinct families: Arenaviridae, Bunyaviridae, Filoviridae, Flaviviridae, and Paramyxoviridae. The viruses of these families are all RNA viruses, and are all covered, or enveloped, in a fatty (lipid) coating. The survival of VHFs is dependent on an animal or insect host (the natural reservoir). The viruses are geographically restricted to the areas where their host species live, and humans are infected when they come into contact with infected hosts. With some of the viruses, after transmission from the host, humans can transmit the virus to one another. Human cases or outbreaks of hemorrhagic fevers caused by these viruses occur sporadically and irregularly. The occurrence of outbreaks cannot be easily predicted. With a few exceptions, there is no cure or established drug treatment for VHFs.

VHFs caused by Arenaviruses and Filoviruses together cover a wide geographic region ranging from Western through to Central Africa and threaten adjacent regions where infected animal reservoirs may migrate but where human disease has not yet been reported. Filoviruses encode their genome in the form of single-stranded negative-sense RNA. Two members of the family that are commonly known are Ebola virus and Marburg virus. Ebola is an emerging and re-emerging RNA viral disease. Outbreaks are not always caused by the exact same virus, but by different relatives (types) of the same virus family of which there are close siblings (for example, Ebola Mayinga and Ebola Kikwit), close cousins (Tai Forest and Bundibugyo), distant cousins (Sudan), and distant relatives (Marburg virus). The 2014 Ebola outbreak in West Africa was the largest since the viral disease was first recognised. Arenaviruses are divided into two groups: the Old World and the New World viruses. The differences between these groups are distinguished geographically and genetically. At least eight arenaviruses are known to cause human disease ranging in severity. Aseptic meningitis, a severe human disease that causes inflammation covering the brain and spinal cord, can arise from the Lymphocytic choriomeningitis virus (LCMV) infection. Hemorrhagic fever syndromes are derived from infections such as Guanarito virus (GTOV), Junin virus (JUNV), Lassa virus (LASV), Lujo virus (LUJV), Machupo virus (MACV), Sabia virus (SABV), or Whitewater Arroyo virus (WWAV).

Lassa Fever virus (LASV), Ebola (EBOV) and Marburg (MARV) viruses are the most important haemorrhagic fevers in West and Central Africa. Lassa fever is endemic to Western Africa with estimates ranging between 300,000 to a million infections, with 5,000 deaths per year. Lassa Fever virus (LASV), Ebola (EBOV) and Marburg (MARV) viruses are all containment level 4 pathogens with high human morbidity and mortality for which there are no established cures, and currently there are no licensed vaccines for infections caused by these viruses.

Influenza virus is a member of the Orthomyxoviridae family. There are three types of influenza viruses, designated influenza A, influenza B, and influenza C. Influenza A viruses infect a wide variety of birds and mammals, including humans, horses, marine mammals, pigs, ferrets, and chickens. In animals, most influenza A viruses cause mild localized infections of the respiratory and intestinal tract. However, highly pathogenic influenza A strains, such as H5N1, cause systemic infections in poultry in which mortality may reach 100%. In 2009, H1N1 influenza was the most common cause of human influenza. A new strain of swine-origin H1N1 emerged in 2009 and was declared pandemic by the World Health Organization. This strain was referred to as “swine flu”. H1N1 influenza A viruses were also responsible for the Spanish flu pandemic in 1918, the Fort Dix outbreak in 1976, and the Russian flu epidemic in 1977-1978. There are currently two influenza vaccine approaches licensed in the United States-the inactivated, split vaccine and the live-attenuated virus vaccine. The inactivated vaccines can efficiently induce humoral immune responses but generally only poor cellular immune responses. Live virus vaccines cannot be administered to immunocompromised or pregnant patients due to their increased risk of infection.

There is a need, therefore, to provide effective vaccines that induce a broadly neutralising immune response to protect against emerging and re-emerging diseases, especially those caused by viruses such as RNA viruses, including VHFs and influenza.

According to the invention there is provided a method for identifying a lead candidate optimized antigenic pathogen polypeptide capable of inducing a broadly neutralizing immune response to a pathogen, which comprises:
i) providing a polypeptide library comprising a plurality of different candidate optimized antigenic pathogen polypeptides, wherein the amino acid sequence of each different candidate has been optimized from a plurality of different amino acid sequences of a pathogen polypeptide and is different from each different amino acid sequence of the pathogen polypeptide, wherein each different amino acid sequence of the pathogen polypeptide comprises amino acid sequence of a polypeptide of a different isolate, and wherein each different isolate is an isolate of a pathogen of the same family as the pathogen to which it is desired to induce a broadly neutralizing immune response;
ii) screening the candidate optimized antigenic pathogen polypeptides of the polypeptide library for binding by one or more broadly neutralizing antigen-binding molecules, each of which is able to bind and/or neutralize a pathogen of the same family as the pathogen to which it is desired to induce a broadly neutralizing immune response; and iii) identifying a candidate optimized antigenic pathogen polypeptide that is bound by one or more of the antigen-binding molecules in step (ii) as being a lead candidate optimized antigenic pathogen polypeptide capable of inducing a broadly neutralizing immune response to the pathogen.

Optionally each different isolate, or each of a plurality of different isolates, of the pathogen is of the same subtype or type as the pathogen to which it is desired to induce a broadly neutralizing immune response.

Optionally each different isolate, or each of a plurality of different isolates, of the pathogen is of the same species or genus as the pathogen to which it is desired to induce a broadly neutralizing immune response.

Optionally the different isolates include isolates of different subtypes or types within the same family as the pathogen to which it is desired to induce a broadly neutralizing immune response.

Optionally the different isolates include isolates of different species or genera within the same family as the pathogen to which it is desired to induce a broadly neutralizing immune response.

The term “pathogen” is used herein to refer to anything that can cause disease, and in particular to an infectious agent, such as a virus, bacterium, fungus, or parasite, that can cause disease.

The term “polypeptide” is used herein to refer to a polymer comprising a plurality of amino acid residues linked together by peptide bonds to form a chain. All proteins are polypeptides. The term “polypeptide” is used interchangeably with the term “protein”. The term “polypeptide” is specifically intended to cover naturally occurring proteins, as well as those which are recombinantly or synthetically produced. Optionally the polypeptide is a modified polypeptide, such as co-translationally or post-translationally modified polypeptide, for example a glycosylated polypeptide or a glycosylated protein (a “glycoprotein”). Glycoproteins are proteins which contain oligosaccharide chains (glycans) covalently attached to amino acid side-chains. The carbohydrate is attached to the protein by co-translational or post-translational glycosylation.

A “pathogen polypeptide” refers to any polypeptide forming part of a pathogen. Optionally the pathogen polypeptide is a structural protein (or portion thereof) of the pathogen. Optionally the pathogen polypeptide is a structural protein (or portion thereof) that is exposed on the surface of the pathogen. Optionally the pathogen polypeptide is a viral protein (or portion thereof). Optionally the pathogen polypeptide is a viral envelope protein (or portion thereof). Optionally the pathogen polypeptide is a glycoprotein (or portion thereof). Optionally the pathogen polypeptide is a viral glycoprotein (or portion thereof). Optionally the pathogen polypeptide is a viral envelope glycoprotein (or portion thereof). Optionally the pathogen polypeptide is an external viral envelope glycoprotein (or portion thereof). Optionally a pathogen polypeptide comprises an amino acid sequence of at least 20 amino acid residues. Optionally a pathogen polypeptide comprises an amino acid sequence of upto 1000, 900, 800, 700, or 600 amino acid residues.

A fully assembled infectious virus is known as a virion. The simplest virions consist of nucleic acid (single- or double-stranded RNA or DNA) and a capsid protein coat. Capsids are formed as single or double protein shells and consist of only one or a few structural protein species. Enveloped viruses have envelopes covering their protective protein capsids. The envelopes are typically derived from portions of the host cell membranes (phospholipids and proteins), but include virus-encoded glycoproteins.

Glycoproteins on the surface of the envelope serve to identify and bind to receptor sites on the host's membrane. The viral envelope then fuses with the host's membrane, allowing the capsid and viral genome to enter and infect the host. Virus-cell membrane fusion is the means by which all enveloped viruses, including human pathogens such as filovirus, influenza virus, and human immunodeficiency virus (HIV), enter cells and initiate virus infection. This membrane fusion process is executed by one or more viral envelope glycoproteins. The fusion can occur on the cell plasma membrane or endosomal membrane.

Glycoproteins may help viruses avoid the host immune system. Enveloped viruses possess great adaptability, and can change in a short time to evade the host immune system. Enveloped viruses can cause persistent infections. Enveloped RNA viruses include, for example, Flavivirus, Togavirus, Coronavirus, Hepatitis D, Orthomyxovirus, Paramyxovirus, Rhabdovirus, Bunyavirus, Filovirus. Retroviruses are enveloped viruses. Enveloped DNA viruses include Herpesviruses, Poxviruses, Hepadnaviruses.

Most external viral envelope proteins are glycoproteins, occurring as membrane-anchored spikes, often assembled as dimers or trimers. The trimeric glycoprotein (GP) spike on the envelope of filoviruses mediates all stages of virus entry, including attachment, entry, and fusion. Recognition sites for cellular receptors are often located at the furthest domain from the viral envelope (distal end) whereas proximal domains interact with the lipid bilayer of the envelope. Oligosaccharide side-chains (glycans) are attached by N-glycosidic, or more rarely O-glycosidic, linkages. Since these are synthesized by cellular glycosyl transferases, the sugar composition of these glycans is analogous to that of host cell membrane glycoproteins.

Entry of filoviruses on the cell surface has been shown to be mediated by host cell attachment factors such as C-type lectins, including DC-SIGN (dendritic-cell-specific ICAM3-grabbing non-integrin; also known as CD209) and L-SIGN (liver and lymph node SIGN; also known as CLEC4M) and several cell-surface proteins such as integrins, T cell immunoglobulin and mucin domain-containing (TIM) proteins, and tyrosine protein kinase receptor 3 (TYRO3) family members. Following binding to the cell surface, filoviruses are internalized by a macropinocytosis-like process and subsequently trafficked through early and late endosomes. The viral genome then penetrates into the cytoplasm after fusion of the viral envelope with the membrane of the late endosome. In the cytoplasm, the viral genome is replicated and transcribed, and new viral proteins are synthesized to assemble progeny virions, which bud from the cell surface.

The surface glycoprotein, GP, of Ebola virus (EBOV) is a key component of many vaccines and a target of neutralizing antibodies. The EBOV GP is synthesized as a single polypeptide that is subsequently cleaved by furin-like proteases into GP1 and GP2 subunits, which remain together through an inter-subunit disulfide bond and non-covalent interactions, and form a trimer of GP1-GP2 heterodimers on the viral surface. Furin cleavage, however, is not sufficient to prime EBOV GP. After entering the cell, the virus is eventually trafficked to late endosomes, where GP is further primed to remove some “cap” components, thereby triggering the induction of the crucial membrane fusion event, which leads to viral penetration. EBOV GP priming is mediated by the cysteine proteases cathepsin B and cathepsin L, which cleave GP1 within the □13-□14 loop. Cathepsin cleavage removes ~60% of the amino acids from GP1, including the mucin-like domain, the glycan cap, and the outmost □ strand of the proposed receptor binding region, resulting in a primed form of GP (named GPcl, the 19 kDa GP1 plus GP2). Unlike the full-length GP, the primed GPcl cannot bind to endosomal membrane protein Niemann-Pick C1 (NPC1), which is an indispensable host entry factor for EBOV infection. The crystal structures of free NPC1-C and its complex with GPcl have been determined (Wang et al., Cell, 2016, 164, 258-268). During Ebola virus infection the primary product of the GP gene is secreted GP (sGP), a soluble dimer that lacks GP2 and the mucin-like domain, but shares 295 amino acids of GP1.

The influenza virion contains a segmented negative-sense RNA genome, which encodes the following proteins: hemagglutinin (HA), neuraminidase (NA), matrix (MI), proton ion-channel protein (M2), nucleoprotein (NP), polymerase basic protein 1 (PB1), polymerase basic protein 2 (PB2), polymerase acidic protein (PA), and non-structural protein 2 (NS2). The HA, NA, M I, and M2 are membrane associated, whereas NP, PB1, PB2, PA, and NS2 are nucleocapsid associated proteins. The M I protein is the most abundant protein in influenza particles. The HA and NA proteins are envelope glycoproteins, responsible for virus attachment and penetration of the viral particles into the cell, and the sources of the major immunodominant epitopes for virus neutralization and protective immunity. Both HA and NA proteins are considered the most important components for prophylactic influenza vaccines.

For bacteria or fungi, suitable pathogen polypeptides include polypeptides that are essential for the propagation of a bacterium or fungus, or for the ability of a bacterium or fungus to infect or cause disease in a human. Suitable examples include surface-expressed polypeptides or proteins (see, for example, Hu et al., Front. Microbiol. 8:82. doi: 10.3389/fmicb.2017.00082; Santos and Levitz, Cold Spring Harb Perspect Med. 2014; 4 (11): a019711).

The term “antigenic” is used herein to refer to a substance that is capable of inducing an immune response in a host organism. The immune response may be humoral and/or a cellular immune response. A cellular immune response is a response of a cell of the immune system, such as a B-cell, T-cell, macrophage or polymorphonucleocyte, to a stimulus such as an antigen or vaccine. An immune response can include any cell of the body involved in a host defence response, including for example, an epithelial cell that secretes an interferon or a cytokine. An immune response includes, but is not limited to, an innate immune response or inflammation. As used herein, a protective immune response refers to an immune response that protects a subject from infection or disease (i.e. prevents infection or prevents the development of disease associated with infection). Methods of measuring immune responses are well known in the art and include, for example, measuring proliferation and/or activity of lymphocytes (such as B or T cells), secretion of cytokines or chemokines, inflammation, or antibody production.

Optionally an optimized antigenic pathogen polypeptide is able to induce the production of antibodies and/or a T-cell response in a human or non-human animal to which the polypeptide has been administered (either as a polypeptide or, for example, expressed from an administered nucleic acid expression vector).

The term “antibody” is used herein to refer to an immunoglobulin molecule produced by B lymphoid cells with a specific amino acid sequence. Antibodies are evoked in humans or other animals by a specific antigen (immunogen). Antibodies are characterized by reacting specifically with the antigen in some demonstrable way, antibody and antigen each being defined in terms of the other. “Eliciting an antibody response” refers to the ability of an antigen or other molecule to induce the production of antibodies.

“Neutralizing” antibodies or antigen-binding molecules not only bind to a pathogen, such as a virus, they bind in a manner that inhibits (i.e. reduces) or blocks infection, or progression of infection. A neutralizing antibody or antigen-binding molecule may block interactions with the receptor, or may bind to a viral capsid in a manner that inhibits uncoating of the genome. The term “neutralizing antibodies” or “neutralizing antigen-binding molecules” also includes antibodies or antigen-binding molecules that are able to prevent infection of a pathogen, such as a virus, by facilitating a cytokine response or by facilitating uptake and removal by an immune cell. In particular, the term “neutralizing antibodies” includes antibodies (or fragments or derivatives thereof) capable of inhibiting or blocking infection (or progression of infection) of a pathogen by antibody-dependent cell-mediated cytotoxicity (ADCC) or complement-dependent cytotoxicity (CDC). Only a small subset of the many antibodies that bind a virus are capable of neutralization.

The term “broadly neutralizing antigen-binding molecule” is used herein to include an antigen-binding molecule, such as an antibody or fragment or derivative thereof, that is able to inhibit (i.e. reduce), neutralize or prevent infection of at least two different subtypes or species of a pathogen, for example at least two different subtypes or species of a virus, at least two different subtypes or species of a bacterium, or at least two different subtypes or species of a fungus. Optionally a broadly neutralizing antigen-binding molecule is able to inhibit (i.e. reduce), neutralize or prevent infection of most or all different subtypes or species of a pathogen, for example most or all different subtypes or species of a virus, most or all different subtypes or species of a bacterium, or most or all different subtypes or species of a fungus. Optionally a broadly neutralizing antibody is able to inhibit (i.e. reduce), neutralize or prevent infection of members of at least two different types of a pathogen (for example a virus, bacterium, or fungus) within the same family.

Optionally a plurality of different broadly neutralizing antigen-binding molecules are used in step (ii) of a method of the invention. Optionally each different broadly neutralizing antigen-binding molecule binds to a different region or epitope of the candidate optimized antigen pathogen polypeptides of the polypeptide library.

The term “broadly neutralizing immune response” is used herein to mean an immune response elicited in a subject that is sufficient to inhibit (i.e. reduce), neutralize or prevent infection, and/or progress of infection, of at least two different subtypes or species of a pathogen, for example at least two different subtypes or species of a virus, at least two different subtypes or species of a bacterium, or at least two different subtypes or species of a fungus. Optionally a broadly neutralizing immune response is sufficient to inhibit, neutralize or prevent infection, and/or progress of infection, of most or all different subtypes or species of a pathogen, for example most or all different subtypes or species of a virus, most or all different subtypes or species of a bacterium, or most or all different subtypes or species of a fungus. Optionally a broadly neutralizing immune response is sufficient to inhibit, neutralize or prevent infection, and/or progress of infection, of members of at least two different types of a pathogen (for example a virus, bacterium, or fungus) within the same family. Optionally a broadly neutralizing immune response is sufficient to inhibit, neutralize or prevent infection, and/or progress of infection, of members of at least two different genera of a pathogen (for example a virus, bacterium, or fungus) within the same family.

Several broadly neutralizing antibodies to pathogens are known. For example, some antibodies have been demonstrated to be capable of neutralizing viral isolates of diverse subtypes across the Filovirus family. A systematic analysis of monoclonal antibodies against Ebola virus glycoprotein is described by Saphire et al. (Cell, 2018; 174 (4): 938-952). An example of a broadly neutralizing antibody to Ebolavirus is immune-elicited macaque antibody CA45, described by Zhao et al., 2017 (Cell 169, 891-904). Broadly neutralizing monoclonal antibodies against the HIV-1 envelope protein are referenced in Bruun et al. (PLOS ONE 9 (10): e109196. doi: 10.1371/journal.pone.0109196) Corti et al., (Curr Opin Virol. 2017 June; 24:60-69) provide an overview of the specificity, antiviral and immunological mechanisms of action and development into the clinic of broadly reactive monoclonal antibodies against influenza A and B viruses.

Optionally the pathogen is a virus.

Viruses are mainly classified by phenotypic characteristics, such as morphology, nucleic acid type, mode of replication, host organisms, and the type of disease they cause. One scheme for the classification of viruses, the Baltimore classification system, places viruses into one of seven groups depending on a combination of their nucleic acid (DNA or RNA), strandedness (single-stranded or double-stranded), sense, and method of replication:
I: dsDNA viruses (e.g. Adenoviruses, Herpesviruses, Poxviruses);
II: ssDNA viruses (+ strand or “sense”) DNA (e.g. Parvoviruses);
III: dsRNA viruses (e.g. Reoviruses);
IV: (+) ssRNA viruses (+ strand or sense) RNA (e.g. Picornaviruses, Togaviruses);
V: (−) ssRNA viruses (− strand or antisense) RNA (e.g. Orthomyxoviruses, Filoviruses, Arenaviruses, Rhabdoviruses);
VI: ssRNA-RT viruses (+ strand or sense) RNA with DNA intermediate in life-cycle (e.g. Retroviruses);
VII: dsDNA-RT viruses DNA with RNA intermediate in life-cycle (e.g. Hepadnaviruses).

Optionally the virus is an RNA virus. RNA viruses comprise:
Group III: viruses possess double-stranded RNA genomes;
Group IV: viruses possess positive-sense single-stranded RNA genomes. Many well known viruses are found in this group, including the picornaviruses (which is a family of viruses that includes well-known viruses like Hepatitis A virus, enteroviruses, rhinoviruses, poliovirus, and foot-and-mouth disease virus), SARS virus, hepatitis C virus, yellow fever virus, and rubella virus;
Group V: viruses possess negative-sense single-stranded RNA genomes. Ebola and Marburg viruses are well known members of this group, along with influenza virus, Lassa virus, measles, mumps and rabies.

Grouping of different RNA virus families under the Baltimore classification is set out in the Table below:

| | Examples | | | | |
|---|---|---|---|---|---|
| | (common | | Capsid | Nucleic | |
| RNA Virus Family | names) | Capsid | Symmetry | acid type | Group |
| Reoviridae | Reovirus, rotavirus | naked/ | Icosahedral | ds | III |
| | | enveloped | | | |
| Picornaviridae | Enterovirus, rhinovirus, | Naked | Icosahedral | ss | IV |
| | hepatovirus, cardiovirus, | | | | |
| | aphthovirus, poliovirus, | | | | |
| | parechovirus, erbovirus, | | | | |
| | kobuvirus, teschovirus, | | | | |
| | coxsackie | | | | |
| Caliciviridae | Norwalk virus | Naked | Icosahedral | ss | IV |
| Togaviridae | Rubella virus, | Naked | Icosahedral | ss | IV |
| | alphavirus | | | | |
| Arenaviridae | Lymphocytic | Enveloped | Complex | ss(−) | V |
| | choriomeningitis virus, | | | | |
| | Lassa virus | | | | |
| Flaviviridae | Dengue virus, hepatitis | Enveloped | Icosahedral | ss | IV |
| | C virus, yellow fever | | | | |
| | virus, Zika virus | | | | |
| Orthomyxoviridae | Influenzavirus A, | Enveloped | Helical | ss(−) | V |
| | influenzavirus B, | | | | |
| | influenzavirus C, | | | | |
| | isavirus, thogotovirus | | | | |
| Paramyxoviridae | Measles virus, mumps | Enveloped | Helical | ss(−) | V |
| | virus, respiratory | | | | |
| | syncytial virus, | | | | |
| | Rinderpest virus, | | | | |
| | canine distemper virus | | | | |
| Bunyaviridae | California encephalitis | Enveloped | Helical | ss(−) | V |
| | virus, hantavirus | | | | |
| Rhabdoviridae | Rabies virus | Enveloped | Helical | aa(−) | V |
| Filoviridae | Ebola virus, Marburg | Enveloped | Helical | ss(−) | V |
| | virus | | | | |
| Coronaviridae | Corona virus | Enveloped | Helical | ss | IV |
| Astroviridae | Astrovirus | Enveloped | Icosahedral | ss | IV |
| Bornaviridae | Borna disease virus | Naked | Helical | ss(−) | V |
| Arteriviridae | Arterivirus, equine | Enveloped | Icosahedral | ss | IV |
| | arteritis virus | | | | |
| Hepeviridae | Hepatitis E virus | Enveloped | Icosahedral | ss | IV |

Optionally the virus is an emerging or re-emerging RNA virus. Examples of emerging or re-emerging RNA viruses include Ebola virus, Marburg virus, Lassa virus, Influenza virus, MERS coronavirus, Hendra virus, Nipah virus.

Optionally the virus is a Filovirus or an Arenavirus. Optionally the virus is Ebola virus or Marburg virus. Optionally the virus is Lassa virus. Optionally the virus is influenza virus.

Optionally the pathogen is a DNA virus. Optionally the pathogen is a member of the Poxviridae family, for example monkey pox virus.

DNA viruses comprise:

Group I: viruses possess double-stranded DNA. Viruses that cause chickenpox and herpes are found here.

Group II: viruses possess single-stranded DNA.

Grouping of different DNA virus families under the Baltimore classification is set out in the Table below:

| | Examples | Capsid: | | | |
|---|---|---|---|---|---|
| | (common | naked/ | Capsid | Nucleic | |
| DNA Virus family | names) | enveloped | symmetry | acid type | Group |
| Adenoviridae | Adenovirus, | Naked | Icosahedral | ds | I |
| | infectious canine | | | | |
| | hepatitis virus | | | | |
| Papovaviridae | Papillomavirus, | Naked | Icosahedral | ds circular | I |
| | polyomaviridae, | | | | |
| | simian | | | | |
| | vacuolating virus | | | | |
| Parvoviridae | Parvovirus B19, | Naked | Icosahedral | ss | II |
| | canine parvovirus | | | | |
| Herpesviridae | Herpes simplex | Enveloped | Icosahedral | ds | I |
| | virus, varicella- | | | | |
| | zoster virus, | | | | |
| | cytomegalovirus, | | | | |
| | Epstein-Barr virus | | | | |
| Poxviridae | Smallpox virus, | Complex coats | Complex | ds | I |
| | cow pox virus, | | | | |
| | sheep pox virus, | | | | |
| | orf virus, monkey | | | | |
| | pox virus, vaccinia | | | | |
| | virus | | | | |
| Hepadnaviridae | Hepatitis B virus | Enveloped | Icosahedral | circular, | VII |
| | | | | partially ds | |
| Asfarviridae | African swine | Envelopes | Icosahedral | ds | I |
| | fever virus | | | | |

Optionally the pathogen is a reverse transcribing virus. Reverse transcribing viruses comprise:
Group VI: viruses possess single-stranded RNA viruses that replicate through a DNA intermediate. The retroviruses are included in this group, of which HIV is a member.
Group VII: viruses possess double-stranded DNA genomes and replicate using reverse transcriptase. The hepatitis B virus can be found in this group.

The term “subtype” is used herein to refer to a genetic variant, or strain, of a pathogen (for example, a virus, bacterium, or fungus). For example, the genus Ebolavirus is a virological taxon included in the family Filoviridae. The members of this genus are called ebolaviruses. The six known ebolavirus subtypes are named for the region where each was originally identified: Bundibugyo, Reston, Sudan, Taï Forest, Zaire, and Bombali. Influenza A viruses are divided into subtypes on the basis of two proteins on the surface of the virus: hemagglutinin (HA) and neuraminidase (NA). There are 18 known HA subtypes and 11 known NA subtypes. Many different combinations of HA and NA proteins are possible. For example, an “H7N2 virus” designates an influenza A virus subtype that has an HA 7 protein and an NA 2 protein. Similarly an “H5N1” virus has an HA 5 protein and an NA 1 protein.

Virus nomenclature for natural variants of the family Filoviridae is discussed in Kuhn et al. (Arch Virol. 2013 January; 158 (1): 301-311). According to the authors a (natural) virus strain is a “variant of a given virus that is recognizable because it possesses some unique phenotypic characteristics that remain stable under natural conditions”. Such “unique phenotypic characteristics” are biological properties different from the compared reference virus, such as unique antigenic properties, host range or the signs of disease it causes. A virus variant with a simple “difference in genome sequence . . . is not given the status of a separate strain since there is no recognizable distinct viral phenotype”. A strain is therefore a genetically stable virus variant that differs from a natural reference virus (type variant) in that it causes a significantly different, observable, phenotype of infection (different kind of disease, infecting a different kind of host, being transmitted by different means etc.). “Genetically stable” means that the genomic changes associated with the phenotypic change are largely preserved over time through natural selection. The extent of genomic sequence variation is irrelevant for the classification of a variant as a strain since a distinct phenotype sometimes arises from few mutations. “Observable phenotype” means, for instance, that within a comparative animal experiment, it would be possible for the researcher to distinguish between the reference control virus-infected animal and the animal infected with the alleged new strain, without knowing which animal received which virus and without having any information about the differences between the two viruses. The designation of a virus variant as a virus strain is the responsibility of international expert groups. Thus far, natural filovirus strains according to this definition have not been reported. All described genetic variants of EBOV, for instance, cause a similar haemorrhagic fever in humans and even experimental animals and are transmitted similarly. None of the known EBOV genetic variants can be distinguished from others on clinical grounds alone. In fact, their variety seems to be limited to subtle differences in growth kinetics and plaque formation in vitro or subtle changes in the duration of disease in experimental animals, and ultimately derives from limited, but often stable, differences in genomic sequence. This also holds true for the different genetic variants of MARV, RAVV, BDBV, RESTV, and SUDV (currently, there is only one isolate of TAFV and none of LLOV).

According to Kuhn et al., a natural genetic filovirus variant is a natural filovirus that differs in its genomic consensus sequence from that of a reference filovirus (the type virus of a particular filovirus species) by ≤10% but is not identical to the reference filovirus and does not cause an observable different phenotype of disease (filovirus strains would be genetic filovirus variants, but most genetic filovirus variants would not be filovirus strains if a strain definition would be brought forward).

Another scheme for classification of viruses is the International Committee on Taxonomy of Viruses (ICTV) system. The system shares many features with the classification system of cellular organisms, such as taxon structure. However, this system of nomenclature differs from other taxonomic codes on several points. Viral classification starts at the level of order and continues as follows, with the taxon suffixes given in italics:

```
Order (-virales)
  Family (-viridae)
    Subfamily (-virinae)
      Genus (-virus)
        Species
```

Species names often take the form of [Disease] virus, particularly for higher plants and animals.

The establishment of an order is based on the inference that the virus families it contains have most likely evolved from a common ancestor. The majority of virus families remain unplaced. As of 2017, 9 orders, 131 families, 46 subfamilies, 803 genera, and 4,853 species of viruses have been defined by the ICTV. The orders are the Caudovirales, Herpesvirales, Ligamenvirales, Mononegavirales, Nidovirales, Ortervirales, Picornavirales, Bunyavirales and Tymovirales. These orders span viruses with varying host ranges.
Caudovirales are tailed dsDNA (group I) bacteriophages.
Herpesvirales contain large eukaryotic dsDNA viruses.
Ligamenvirales contains linear, dsDNA (group I) archaean viruses.
Mononegavirales include nonsegmented (−) strand ssRNA (Group V) plant and animal viruses.
Nidovirales are composed of (+) strand ssRNA (Group IV) viruses with vertebrate hosts.
Ortervirales contain single-stranded RNA and DNA viruses that replicate through a DNA intermediate (Groups VI and VII).
Picornavirales contains small (+) strand ssRNA viruses that infect a variety of plant, insect and animal hosts.
Tymovirales contain monopartite (+) ssRNA viruses that infect plants.
Bunyavirales contain tripartite (−) ssRNA viruses (Group V).

According to the ICTV, a virus species is “a monophyletic group of viruses whose properties can be distinguished from those of other species by multiple criteria.”

The term “isolate” is used herein to refer to a pure pathogen sample that has been obtained from an infected individual. A virus-infected cell will, after only one round of replication, already contain a population of genomes, and virions derived from these genomes will vary slightly from each other. Likewise, a sample taken from an infected individual will contain numerous virions, many of which vary slightly. Consequently, an “isolate” refers to a population, and “the sequence” of an “isolate” is a consensus sequence of the population of genomes present in the analyzed sample. A virus isolate may be defined as “an instance of a particular virus”. A natural filovirus isolate is an instance of a particular natural filovirus or of a particular genetic variant. Isolates can be identical or slightly different in consensus or individual sequence from each other.

Optionally the one or more broadly neutralizing antigen-binding molecules include an antibody that has been obtained, or derived from an antibody that has been obtained, from a subject that has been exposed to a pathogen of the same family as the pathogen to which it is desired to induce a broadly neutralizing immune response.

Optionally the one or more broadly neutralizing antigen-binding molecules include an antibody that has been obtained, or derived from an antibody that has been obtained, from a subject that has been exposed to a pathogen of the same subtype or type as the pathogen to which it is desired to induce a broadly neutralizing immune response.

Optionally the one or more broadly neutralizing antigen-binding molecules include an antibody that has been obtained, or derived from an antibody that has been obtained, from a subject that has been exposed to a pathogen of the same species or genus as the pathogen to which it is desired to induce a broadly neutralizing immune response.

Optionally the one or more broadly neutralizing antigen-binding molecules include non-antibody antigen-binding proteins. For example, the one or more broadly neutralizing antigen-binding molecules may include a designed ankyrin repeat protein (DARPin), an aptamer, an anticalin, or a T-cell receptor molecule.

DARPins are genetically engineered antibody mimetic proteins typically exhibiting highly specific and high-affinity target protein binding. They are derived from natural ankyrin proteins, and comprise repetitive structural units that form a stable protein domain with a large potential target interaction surface. Typically, DARPins comprise four or five repeats, of which the first (N-capping repeat) and last (C-capping repeat) serve to provide a hydrophilic surface. DARPins correspond to the average size of natural ankyrin repeat protein domain. Proteins with fewer than three repeats (i.e., the capping repeats and one internal repeat) do not form a stable enough tertiary structure. The molecular mass of a DARPin depends on the total number of repeats:

| | | | | | | |
|---|---|---|---|---|---|---|
| Repeats | 3 | 4 | 5 | 6 | 7 | . . . |
| ~Mass (kDa) | 10 | 14 | 18 | 22 | 26 | . . . |

Libraries of nucleic acids encoding DARPins with randomized potential target interaction residues, with diversities of over 10¹² variants, can be generated. From these libraries, DARPins can be selected to bind to a desired target of choice with picomolar affinity and specificity using ribosome display or phage display using signal sequences allowing co-translational secretion. Thus, by screening a library of DARPins, one or more DARPins can be identified that bind and/or neutralize more than one subtype of pathogen. Library-based screening for the identification of DARPins is described, for example, in Hartmann et al. (Molecular Therapy: Methods and Clinical Development 2018 Vol. 10:128-143).

Optionally the one or more antigen-binding molecules recited in step (ii) of a method of the invention include a broadly neutralizing antibody (or a fragment or derivative thereof that retains broadly neutralizing activity), for example a broadly neutralizing monoclonal antibody (BNmAb) (or a fragment or derivative thereof that retains broadly neutralizing activity).

Optionally the one or more antigen-binding molecules recited in step (ii) of a method of the invention include an antibody obtained, or derived from an antibody obtained, from a subject that has survived an outbreak of a pathogen of the same subtype, type, or family as the pathogen to which it is desired to induce a broadly neutralizing immune response.

Optionally the one or more antigen-binding molecules recited in step (ii) of a method of the invention include an antibody obtained, or derived from an antibody obtained, from a subject that has survived an outbreak of a pathogen of the same species, genera, or family as the pathogen to which it is desired to induce a broadly neutralizing immune response.

The term “outbreak” is used herein to refer to the occurrence of more cases of a disease than would normally be expected in a defined institution (e.g. a hospital or a medical treatment centre), community, geographical area, or period of time. An outbreak may occur in a restricted geographical area, or may extend over several countries. It may last for a few days or weeks, or for several years. The number of cases indicating presence of an outbreak will vary according to the pathogen, size and type of population exposed, previous experience or lack of exposure to the disease, and time and place of occurrence. Therefore, the status of an outbreak is relative to the usual frequency of the disease in the same area, among the same community, at the same season of the year. The existence of an outbreak may be established by comparing current information with previous incidence in the population or community during the same time of year to determine if the observed number of cases exceeds the expected number.

Optionally an outbreak of a pathogen may refer to the occurrence of more cases of a disease caused by the pathogen than would normally be expected in a region (for example a continental region) or country, or in a population or community, over one or more seasons or over a year.

Optionally an outbreak of a pathogen (such as a virus) is the occurrence of more cases of a disease caused by the pathogen than would normally be expected in a region (for example a continental region) over a season.

Optionally an outbreak of a pathogen (such as a virus) is the occurrence of more cases of a disease caused by the pathogen than would normally be expected in a population over a season.

Examples of continental regions include regions of Africa:
Northern Africa: Algeria; Canary Islands; Ceuta; Egypt; Libya; Madeira; Melilla; Morocco; Sudan; Tunisia; Western Sahara;
Eastern Africa: Burundi; Comoros; Djibouti; Eritrea; Ethiopia; Kenya; Madagascar; Malawi; Mauritius; Mayotte; Mozambique; Reunion; Rwanda; Seychelles; Somalia; South Sudan; Tanzania; Uganda; Zambia; Zimbabwe;
Central Africa: Angola; Cameroon; Central African Republic; Chad; Democratic Republic of the Congo; Republic of the Congo; Equatorial Guinea; Gabon; São Tomé and Príncipe;
Western Africa: Benin; Burkina Faso; Cape Verde; Ivory Coast; Gambia; Ghana; Guinea; Guinea-Bissau; Liberia; Mali; Mauritania; Niger; Nigeria; Saint Helena; Senegal; Sierra Leone; Togo;
Southern Africa: Botswana; Lesotho; Namibia; South Africa; Swaziland.

Optionally the subject from which the antibody has been obtained or derived is a human or non-human mammalian subject.

The candidate optimized antigenic pathogen polypeptides of the polypeptide library may have been expressed using any suitable expression system. Suitable examples include mammalian cells, or yeast or insect or bacterial cells.

Optionally the candidate optimized antigenic pathogen polypeptides of the polypeptide library are expressed on the surface of a cell of the expression system. Cell surface expression increases the likelihood that the candidate optimized antigenic pathogen polypeptides are correctly folded.

Optionally the candidate optimized antigenic pathogen polypeptides are screened for binding by the one or more antigen-binding molecules by flow cytometry. For example, cells expressing the candidate optimized antigenic pathogen polypeptides may be used in a flow cytometry assay.

Optionally the candidate optimized antigenic pathogen polypeptides are screened for binding by one or more broadly neutralizing antigen-binding molecules using a first assay (such as flow cytometry) and for binding by one or more broadly neutralizing antigen-binding molecules using a second assay (such as a neutralization assay).

Optionally the pathogen is a virus, the candidate optimized antigenic pathogen polypeptides are candidate optimized antigenic virus polypeptides, and the pathogen peptides are virus polypeptides.

Optionally the polypeptide library is a viral pseudotype library comprising a plurality of different viral pseudotypes, each different viral pseudotype comprising a different candidate optimized antigenic pathogen polypeptide, for example a different candidate optimized antigenic virus polypeptide (such as a viral glycoprotein).

Optionally, in step (ii), the candidate optimized antigenic virus polypeptides are screened for binding by one or more of the broadly neutralizing antigen-binding molecules by screening the viral pseudotypes for binding and/or neutralization by one or more of the antigen-binding molecules.

Pseudotyping is the process of producing viruses or viral vectors in combination with foreign viral envelope proteins. The result is a pseudotyped virus particle.

Pseudotyped particles do not carry the genetic material to produce additional viral envelope proteins, so the phenotypic changes cannot be passed on to progeny viral particles. A “pseudotype” may be defined as a hybrid virus particle comprising a protein nucleocapsid (‘core’) encasing a nucleic acid (RNA or DNA) genome, with the core itself being encapsulated in a lipid ‘envelope’ membrane derived from the host cell. This envelope gained when cores exit from the cell by ‘budding’ includes proteins derived from other viruses. Many of these heterologous envelope proteins are antigenic targets for the host immune system. In pseudotypes, one or more of these envelope proteins may derive from study viruses. Many pseudotypes also carry foreign genes, called ‘transfer’ genes, engineered into their genome. When in the presence of susceptible cells, the envelope proteins bind to cell receptors permitting cellular entry, eventually resulting in transfer gene expression. Rhabdoviruses (e.g. Vesticular Stomatitis Virus, VSV) and Retroviruses (e.g. Lentiviruses) have been extensively exploited as cores for pseudotyping. In the case of retroviruses, their key characteristic is the ability to reverse transcribe their dimeric single-stranded RNA genome into a double-stranded deoxyribonucleic acid (dsDNA) copy, which is subsequently integrated into the cell genome via the use of viral and cellular enzymes. For retroviral pseudotypes, this usually leads to expression of the transfer/reporter gene, the latter being readily quantifiable. Reporter gene expression directly correlates with efficiency of viral envelope/receptor interaction, and conversely whether individual antibody responses or antiviral agents could interfere with the entry and replication process of the native virus.

Binding of viral pseudotypes to broadly neutralizing antigen-binding molecules may be measured using any suitable technique known to the skilled person, for example by haemagglutination inhibition (HI) assay, or by enzyme-linked immunosorbent assay (ELISA). ELISA analysis of antibody binding to glycoprotein (GP) is described in Saphire et al., 2018 (Cell 174 (4): 938-952) in relation to analysis of monoclonal antibodies against Ebola virus GP.

Production of retroviral pseudotypes, and their use in pseudotype neutralisation assays and immunogenicity testing, is reviewed in detail in Temperton et al., 2015 (Retroviral Pseudotypes—From Scientific Tools to Clinical Utility. In: eLS. John Wiley & Sons, Ltd: Chichester. DOI: 10.1002/9780470015902.a0021549.pub2).

Representatives of all seven genera of retroviruses have been employed in pseudotyping studies but to date only gammaretroviral or lentiviral pseudotypes are widely used. Lentiviruses are a genus of the Retroviridae family, which unlike gammaretroviruses, can infect non-proliferating cells, which makes them amenable for gene therapy applications involving highly differentiated or quiescent cells (e.g. in Go cell cycle phase) including muscle or neurons. The most common lentivirus vector used for pseudotyping is HIV-type 1 (HIV-1), although simian immunodeficiency virus has also been employed.

Generation of retroviral pseudotypes is achieved through the introduction of cloned versions of foreign envelope protein gene(s), core retroviral genes and transfer gene (e.g. reporter or therapeutic gene) concurrently into producer cells, normally highly transfectable cell lines such as human embryo kidney (HEK) 293 clone 17 T cells (American Type Culture Collection #CRL-11268) (Pear et al., 1993, PNAS USA 90:8392-8396).
1. The envelope plasmid. Envelope gene(s) of the study virus are cloned into an appropriate expression plasmid. Genes are usually derived via polymerase chain reaction amplification of viral cDNA using specific primers or from custom gene synthesis. Some expression vectors are commercially available and utilise different, usually strong constitutive gene promoters (e.g. from the human cytomegalovirus (CMV) immediate early gene), which can influence the efficacy of pseudotype generation.
2. The retroviral gag-pol plasmid. The gag and pol genes encode polyproteins which are subsequently cleaved to release structural proteins (including matrix, capsid and nucleocapsid) found within the core, and proteins involved in viral replication (protease, reverse transcriptase and integrase) responsible for processing the structural proteins, converting the ssRNA viral genome into dsDNA and ensuring integration (of the transfer gene) into the host cell genome. In addition, in a lentiviral gag-pol construct, the rev gene is included. The Rev protein is involved in the export of viral mRNAs from nucleus to cytosol for translation.
3. The transfer/reporter plasmid. This is the gene that is stably integrated into the host cell DNA, from where the gene is expressed via various cis-acting transcriptional elements. The transfer plasmid contains a packaging signal upstream of the gene to ensure incorporation of viral RNA containing the gene into the viral core during pseudotype generation.

Once the cellular machinery has transcribed and translated the transfected genes, an RNA dimer of the transfer gene (region between the long terminal repeats; LTR) is incorporated into the pseudotype via the packaging signal. As the transfer plasmid is the only one engineered to contain a packaging signal, no other nucleic acids are incorporated into the mature pseudotype particle. A domain at the N-terminus of Gag targets the nucelocapsid to the cell plasma membrane, into which the envelope protein(s) has been inserted. The pseudotype particles budding from the cell are encapsulated in the cell membrane, which forms the viral envelope.

Pseudotyped viruses are released into the producer cell culture medium. This supernatant can be titrated onto target cells to measure the concentration of functional particles. These attach to the cells via envelope protein-receptor interaction, followed by membrane fusion and internalisation. The pseudotype genome, bearing the transfer/reporter gene is integrated into the host cell DNA, from where it is expressed. The level of reporter gene expression correlates with the level of transduction by viable particles. As only the transfer gene is present in the pseudotype, no viral proteins are produced in target cells, so further pseudotype production and propagation does not occur. This provides safety in working with pseudotypes compared to working with the wildtype virus. Green fluorescent protein (GFP)-based pseudotypes are readily titrated using fluorescence microscopy or flow cytometry, luciferase pseudotypes by luminometry, and beta-galactosidase (β-gal) pseudotypes by colour reaction.

Many standard serological assays measure only antibody binding (hemagglutination inhibition (HI) and ELISA), rather than the inhibition of viral infectivity. Neutralisation assays allow for sensitive detection of functional antibody responses. For high-containment viruses (such as Ebola), however, these assays are not widely applicable owing to the requirement for high biosafety laboratory facilities and specially trained personnel. Using retroviral and lentiviral particles pseudotyped with the envelopes of such pathogens as ‘surrogate viruses’ for use in neutralisation assays is one way of circumventing this issue. Using a pseudotype strategy, only the envelope protein(s) of the virus is required, with no possibility of recombination or native virus escape. These pseudotypes undergo abortive replication and are unable to give rise to replication-competent progeny.

Pseudotypes are excellent serological reagents for virus neutralisation assays as the virions can contain a reporter gene and bear heterologous viral envelope proteins on the surface. The transfer of these reporter genes to target cells depends on the function of the viral envelope protein; therefore, the titre of neutralising antibodies against the envelope can be measured by a reduction in reporter gene transfer and expression. PV neutralisation assays have now been developed for a wide range of RNA viruses, from numerous virus families (see Table 1 of Temperton et al., supra).

Pseudotype-based influenza neutralisation assays have been shown to be highly efficient for the measurement of broadly-neutralising antibodies making them ideal serological tools for the study of cross-reactive responses against multiple subtypes with pandemic potential (Corti et al., 2011, Science 333 (6044): 850-856).

Production of lentiviral vectors pseudotyped with filoviral glycoproteins is described in Sinn et al., 2017 (Methods Mol Biol. 2017; 1628:65-78).

An example of a suitable general method for production of viral pseudotypes is as follows:

For transfection, 5×10⁶ HEK-293T cells are plated 24 h prior to addition of a complex comprising plasmid DNA and PEI, which facilitates DNA transport into the cells. A retroviral gag-pol plasmid and a reporter plasmid are transfected concurrently with the required envelope plasmid.

An example of a suitable neutralization assay is as follows:

In a 96-well plate, ~100×TCID50 pseudotyped virus that resulted in an output of 1×10⁵ relative light units (RLU) is incubated with dilutions of sera for 1 h at 37% (5% CO₂) before the addition of 1×10⁴ target cells. These are incubated for a further 48 h, after which the media is removed and replaced with a 50:50 mix of fresh media and luciferase reagent. Luciferase activity is detected 2.5 min later by reading the plates on a luminometer. For all results, background RLU (virus alone or DEnv) is deducted before analysis.

Saphire et al. (supra) describes three independent assays for evaluation of mAb neutralization in relation to analysis of monoclonal antibodies against Ebola virus GP:
biologically contained EBOV (ΔVP30) (Halfmann et al., 2008, Proc Natl Acad Sci USA. 2008; 105:1129-1133); and
authentic EBOV performed under BSL-2+, BSL-3 and BSL-4 containment; and
replication-competent vesicular stomatitis virus bearing EBOV GP (rVSV).

### Neutralization of EbolaAVP30-RenLuc Virus

An Ebola virus in which the reporter gene Renilla luciferase is substituted for the viral transcription factor VP30 (EbolaΔVP30-RenLuc virus) is used to complement a Vero cell line that stably expresses VP30 in trans (Vero VP30), thus allowing analysis at BSL-3 (Halfmann et al., 2008). A total of 5×10³ focus forming units of EbolaΔVP30-RenLuc virus diluted in 2% fetal calf serum in minimal essential medium is incubated with 50 μg/ml monoclonal antibody for 3 hours at 37° C. The virus/antibody mixture at a multiplicity of infection (MOI) of 0.001 is then added to Vero VP30 cells, seeded the previous day in 96-well plates at 9×10³ cells/well and incubated for three days at 37° C. and 5% CO₂. If used, guinea pig complement (Cedarlane) is added to the minimal essential medium at a final concentration of 10%. Then a live cell luciferase substrate, EnduRen (Promega), is incubated with the cells for three hours before luciferase values are measured as relative light units (RLU) using a Tecan M1000 plate reader (Tecan). Assays are performed in duplicate and a known neutralizing (GP 133/3.16) and non-neutralizing monoclonal (VP35 5/69.3.2) is used as a positive and negative control, respectively. Antibodies that neutralized luciferase signals by ≥95% are defined as strong neutralizers, whereas inhibition of luciferase signals by 50%-94% are considered moderate neutralizers and those that have 49% or lower inhibition are categorized as weak/non-neutralizers.

### Neutralization of Authentic EBOV

Assays to assess neutralization of authentic EBOV are performed according to the method described in Holtsberg et al. (Holtsberg et al., 2015, J Virol. 2015; 90:266-278). Vero E6 cells are seeded 2.5×10⁻⁴ cells/well in the inner 60 wells of black 96-well plates 24 hours prior to virus infection. Antibodies are serially diluted in Vero growth medium (Eagle minimum essential medium with Earle's salts and L-glutamine, 5% fetal bovine serum (FBS) and 1% penicillin-streptomycin) at two times the desired final concentration (50 μg/ml), mixed with an equal volume of live EBOV, and incubated for 1 hour at 37° C. with mixing every 15 min. The antibody/virus mixture at a MOI of 0.2 is then added to the Vero cells and incubated for 1 hr at 37° C., washed with PBS, and growth medium alone is added to all wells and the plates are incubated for an additional 48 hr at 37° C. The cells are then fixed with 10% neutral buffered formalin and the percentage of infected cells is determined by an indirect immunofluorescence assay using the EBOV-specific human mAb KZ52 and goat anti-human IgG conjugated to Alexa Fluor 488 (Molecular Probes) as a secondary antibody. Images are acquired at 20 fields/well with a 20× objective lens on an Operetta High Content Imaging System (Perkin-Elmer). Operetta images are analyzed with a customized algorithm built from image analysis functions available in Harmony software (Perkin-Elmer). The percentage of inhibition for each antibody is determined relative to control cells incubated with media alone. Antibodies that reduced the percentage of infected cells by >80% are categorized as strong neutralizers, whereas those that reduced infection by between 50% and 79% and less than 50% are considered as moderate neutralizers and weak/non-neutralizers, respectively.

Neutralization of rVSV-EBOV GP

Recombinant vesicular stomatitis virus (VSV) expressing both eGFP and recombinant surface GP (rVSV-EBOV) in place of VSV G was described previously (Wec et al., 2016, Science; 354:350-354; Wong et al., 2010, Virol.; 84:163-175). For neutralization assays, Vero cells are seeded at 6.0×10⁴ cells/well and cultured overnight in Eagle's minimal essential medium (EMEM) supplemented with 10% fetal bovine serum (FBS) and 100 I.U./ml penicillin and 100 μg/ml streptomycin at 37° C. and 5% CO2. The next day, virus is incubated with serial 3-fold antibody dilutions beginning at 330 nM (~50 μg/ml) in serum-free EMEM for one hour at room temperature before infecting Vero cell monolayers in 96-well plates. The amount of virus used for infection is determined based on titration of viral stock to achieve 35-50% final infection in control wells without antibody (MOI~0.1 infectious units per cell). The virus is incubated with the cells in 50% v/v/EMEM supplemented with 2% FBS, 100 I.U./ml penicillin and 100 μg/ml streptomycin at 37° C. and 5% CO₂ for 14-16 hours before the cells are fixed and the nuclei stained with Hoescht. rVSV infectivity is measured by counting EGFP-positive cells in comparison to the total number of cells indicated by nuclear staining using a Cellinsight CX5 automated microscope and accompanying software (Thermo Scientific). The infection level in control wells lacking antibody is set to 100% and the infection is normalized to that value for each antibody dilution, which are tested in triplicate. The mean value is determined and the full 9-point dilution curve is used to determine the half-maximal inhibitor concentration, IC50 using GraphPad Prism version 6. Antibodies having IC50≤5 nM are considered strong neutralizers whereas antibodies having 5 nM<IC50<50 nM and ≤50 nM are considered moderate neutralizers and weak/non-neutralizers, respectively. The un-neutralized fraction, an indicator of antibody potency, is also determined using antibodies at the highest concentration tested, 330 nM, and measuring the GFP signal relative to that of untreated control cells. Those that reduce the signal by ≥98%, 50-98%, and less than 50% are considered strong, moderate, and weak/non-neutralizers, respectively.

Methods for screening of polypeptide libraries are described in Bruun et al. (PLOS ONE 9 (10): e109196.

Optionally a method of the invention further comprises generating the polypeptide library.

Optionally the polypeptide library is generated by expressing the different candidate optimized antigenic pathogen polypeptides from a nucleic acid library comprising a plurality of different nucleic acids, each different nucleic acid comprising a nucleotide sequence encoding a different candidate optimized antigenic pathogen polypeptide of the polypeptide library.

Optionally the different candidate optimized pathogen polypeptides are expressed in, or on the surface of, mammalian cells. Suitable methods are well-known to those skilled in the art.

Optionally the nucleotide sequence of each different nucleic acid of the nucleic acid library is optimized for expression of the encoded polypeptide in a mammalian cell.

Optionally each different nucleic acid of the nucleic acid library is part of an expression vector for expression of the nucleic acid in a mammalian cell.

Optionally the pathogen is a virus, the candidate optimized antigenic pathogen polypeptides are candidate optimized antigenic virus polypeptides, and the pathogen peptides are virus polypeptides.

Optionally the nucleic acid library is a viral pseudotype vector library, and each different nucleic acid of the library is part of an expression vector for production of a viral pseudotype comprising the encoded virus polypeptide, and the polypeptide library is a viral pseudotype library generated by producing viral pseudotypes from the expression vectors of the viral pseudotype vector library, wherein the viral pseudotype library comprises a plurality of different viral pseudotypes, each different viral pseudotype comprising a different candidate optimized virus polypeptide encoded by a different nucleic acid sequence of the viral pseudotype vector library.

Optionally the viral pseudotype vector library comprises at least 2, 3, 5, 10, 20, 30, 40, 50, 10², 10³, 10⁴, 10⁵, 10⁶, 10⁷, 10⁸, or 10⁹ different members.

Optionally the expression vector is also a vaccine vector.

Examples of vaccine vector include a viral vaccine vector, a bacterial vaccine vector, an RNA vaccine vector, or a DNA vaccine vector.

Viral vaccine vectors use live viruses to carry nucleic acid (for example, DNA or RNA) into human or non-human animal cells. The nucleic acid contained in the virus encodes one or more antigens that, once expressed in the infected human or non-human animal cells, elicit an immune response. Both humoral and cell-mediated immune responses can be induced by viral vaccine vectors. Viral vaccine vectors combine many of the positive qualities of nucleic acid vaccines with those of live attenuated vaccines. Like nucleic acid vaccines, viral vaccine vectors carry nucleic acid into a host cell for production of antigenic proteins that can be tailored to stimulate a range of immune responses, including antibody, T helper cell (CD4⁺ T cell), and cytotoxic T lymphocyte (CTL, CD8⁺ T cell) mediated immunity. Viral vaccine vectors, unlike nucleic acid vaccines, also have the potential to actively invade host cells and replicate, much like a live attenuated vaccine, further activating the immune system like an adjuvant. A viral vaccine vector therefore generally comprises a live attenuated virus that is genetically engineered to carry nucleic acid (for example, DNA or RNA) encoding protein antigens from an unrelated organism. Although viral vaccine vectors are generally able to produce stronger immune responses than nucleic acid vaccines, for some diseases viral vectors are used in combination with other vaccine technologies in a strategy called heterologous prime-boost. In this system, one vaccine is given as a priming step, followed by vaccination using an alternative vaccine as a booster. The heterologous prime-boost strategy aims to provide a stronger overall immune response. Viral vaccine vectors may be used as both prime and boost vaccines as part of this strategy. Viral vaccine vectors are reviewed by Ura et al., 2014 (Vaccines 2014, 2, 624-641) and Choi and Chang, 2013 (Clinical and Experimental Vaccine Research 2013; 2:97-105).

Optionally the viral vaccine vector is based on a viral delivery vector, such as a Poxvirus (for example, Modified Vaccinia Ankara (MVA), NYVAC, AVIPOX), herpesvirus (e.g. HSV, CMV, Adenovirus of any host species), Morbillivirus (e.g. measles), Alphavirus (e.g. SFV, Sendai), Flavivirus (e.g. Yellow Fever), or Rhabdovirus (e.g. VSV)-based viral delivery vector, a bacterial delivery vector (for example, Salmonella, E. coli), an RNA expression vector, or a DNA expression vector.

Optionally the vector is a pEVAC-based expression vector. A pEVAC expression vector is described in more detail in Example 7 below.

In other embodiments, the different candidate optimized antigenic pathogen polypeptides are expressed in, or on the surface of, bacterial, yeast or insect cells.

Optionally a method of the invention further comprises generating the nucleic acid library by synthesising a plurality of different nucleic acids, each different nucleic acid comprising a different nucleotide sequence encoding a different candidate optimized antigenic pathogen polypeptide.

Optionally methods of the invention further comprise: i) obtaining amino acid sequences of the pathogen polypeptide, and/or nucleotide sequences encoding the pathogen polypeptide, of the different pathogen isolates; and ii) generating a plurality of different nucleotide sequences, each different nucleotide sequence encoding a different candidate optimized antigenic pathogen polypeptide, wherein the encoded amino acid sequence of each different candidate optimized antigenic pathogen polypeptide is optimized from the obtained amino acid sequences or encoded amino acid sequences of the pathogen polypeptide, and is different from each of the obtained amino acid sequences or encoded amino acid sequences.

Optionally generation of the plurality of different nucleotide sequences in step (ii) above comprises: carrying out a multiple sequence alignment of the amino acid or nucleotide sequences obtained in step (i) above; identifying from the multiple sequence alignment amino acid sequence or encoded amino acid sequence that is highly conserved between the polypeptides of the different pathogen isolates; and generating a plurality of different nucleotide sequences, each different nucleotide sequence encoding a different candidate optimized antigenic pathogen polypeptide, wherein one or more of the different nucleotide sequences includes sequence encoding a highly conserved amino acid sequence or encoded amino acid sequence identified from the multiple sequence alignment.

An amino acid sequence or an encoded amino acid sequence that is highly conserved between the polypeptides of the different pathogen isolates may be at least 1, 2, 3, 4, 5, 10, 15, 20, 25, 30, 35, 40, 45, 50, 100, 150, 200, 250, 300, 400, 500, 600, 700, or 800 amino acid residues in length.

Optionally the number of amino acid sequences of the pathogen polypeptide, or the number of nucleotide sequences encoding the pathogen polypeptide, of the different pathogen isolates is at least 3, 4, 5, 10, 20, 30, 40, 50, 100, 200, 300, 400, 500, 600, 700, 800, 900, 1000, 10⁶, 10⁹, or 10¹². Typically, the greater the number of sequences that are used for the multiple sequence alignments, the better.

Optionally methods of the invention further comprise: identifying from the multiple sequence alignment amino acid sequence or encoded amino acid sequence that is ancestral amino acid sequence; and including in one or more of the different generated nucleotide sequences sequence encoding an ancestral amino acid sequence identified from the multiple sequence alignment.

Inclusion of one or more nucleotide sequences encoding ancestral amino acid sequence may be advantageous because ancestral amino acid sequence that is highly conserved with extant amino acid sequence is expected to be of structural and/or functional importance for the survival and/or propagation of the pathogen. Also, as pathogen isolates can be extremely diverse (especially, for example, isolates of emerging or re-emerging pathogens, such as emerging or re-emerging RNA viruses), a vaccine designed to work on one patient's pathogen population might not work for a different patient, because the evolutionary distance between these two pathogen populations may be large. However, their most recent common ancestor is closer to each of the two pathogen populations than they are to each other. Thus, a vaccine designed for a common ancestor could have a better chance of being effective for a larger proportion of circulating strains.

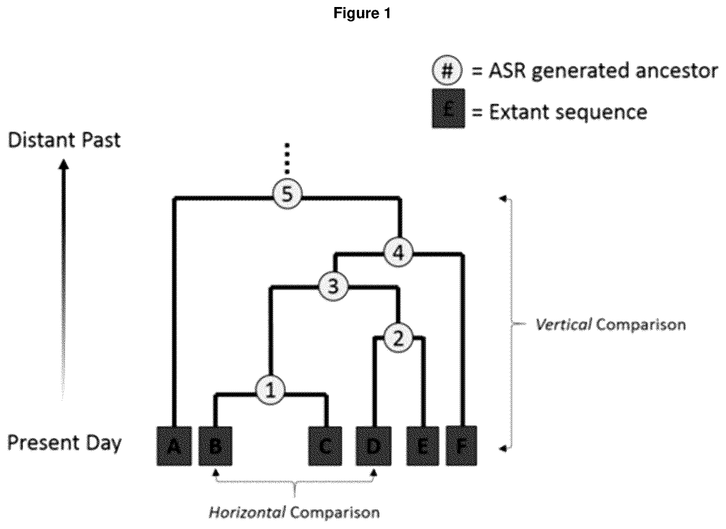

Ancestral sequence reconstruction (ASR) is discussed in Randall et al (Nat. Commun. 7:12847 doi: 10.1038/ncomms 12847 (2016)). The authors reference a definition of ASR as “the process of analyzing modern sequences within an evolutionary/phylogenetic context to infer the ancestral sequences at particular nodes of a tree”. Ancestral sequence reconstruction (ASR) is used in the study of molecular evolution. Unlike conventional evolutionary approaches to studying proteins, by horizontal comparison of related protein homologues from different branch ends of a phylogenetic tree, ASR probes the statistically inferred ancestral proteins within the nodes of the tree in a vertical manner (see FIG. 1). A phylogenetic tree is a branching diagram showing the evolutionary relationships among various biological species or other entities based upon similarities and differences in their physical or genetic characteristics. In a rooted phylogenetic tree, each node with descendants represents the inferred most recent common ancestor of those descendants. In ASR, several related homologues of a protein of interest are selected and aligned in a multiple sequence alignment (MSA), a phylogenetic tree is constructed with statistically inferred sequences at the nodes of the branches. These sequences are the so-called ‘ancestors’. The process of synthesising the corresponding DNA, transforming it into a cell and producing a protein is the so-called ‘reconstruction’.

Ancestral sequences are typically calculated by maximum likelihood, however Bayesian methods are also implemented. Because the ancestors are inferred from a phylogeny, the topology and composition of the phylogeny plays a major role in the output ASR sequences. ASR does not claim to recreate the actual sequence of the ancient protein/DNA, but rather a sequence that is likely to be similar to the one that was at the node. Maximum likelihood (ML) methods work by generating a sequence where the residue at each position is predicted to be the most likely to occupy that position by the method of inference used. Typically, this is a scoring matrix (similar to those used in BLASTs or MSAs) calculated from extant sequences. Alternate methods include maximum parsimony (MP) that construct a sequence based on a model of sequence evolution, usually the idea that the minimum number of nucleotide sequence changes represents the most efficient route for evolution to take and the most likely. MP is often considered the least reliable method for reconstruction as it arguably oversimplifies evolution to a degree that is not applicable on the billion year scale. Other methods include Bayesian methods, which involve the consideration of residue uncertainty. Such methods are sometimes used to compliment ML methods, but typically produce more ambiguous sequences (i.e.

sequences which include residue positions where no clear substitution can be predicted). Often in such cases, several ASR sequences are produced, encompassing most of the ambiguities, and compared to one-another.

Methods and algorithms for ASR are described in more detail below, based on description in Joy et al., 2016, PLOS Computational Biology 12 (7): DOI: 10.1371/journal.pcbi. 1004763.

Optionally ASR is conducted with at least 3, 4, 5, 10, 20, 30, 40, 50, 100, 200, 300, 400, 500, 600, 700, 800, 900, 1000, 10⁶, 10⁹, or 10¹² different sequences. In some instances, the greater the number of sequences that are used, the better.

Optionally each of the sequences used for the multiple sequence alignment is a full length sequence of a pathogen polypeptide of a pathogen isolate.

Any attempt at ancestral reconstruction begins with a phylogeny. In general, a phylogeny is a tree-based hypothesis about the order in which populations (referred to as taxa) are related by descent from common ancestors. Observed taxa are represented by the tips or terminal nodes of the tree that are progressively connected by branches to their common ancestors, which are represented by the branching points of the tree that are usually referred to as the ancestral or internal nodes. Eventually, all lineages converge to the most recent common ancestor of the entire sample of taxa. In the context of ancestral reconstruction, a phylogeny is often treated as though it were a known quantity (with Bayesian approaches being an important exception). Because there can be an enormous number of phylogenies that are nearly equally effective at explaining the data, reducing the subset of phylogenies supported by the data to a single representative, or point estimate, can be a convenient and sometimes necessary simplifying assumption. Ancestral reconstruction can be thought of as the direct result of applying a hypothetical model of evolution to a given phylogeny. When the model contains one or more free parameters, the overall objective is to estimate these parameters on the basis of measured characteristics among the observed taxa (sequences) that descended from common ancestors. Parsimony is an important exception to this paradigm. It is based on the heuristic that changes in character state are rare, without attempting to quantify that rarity.

### Maximum Parsimony

Parsimony refers to the principle of selecting the simplest of competing hypotheses. In the context of ancestral reconstruction, parsimony endeavours to find the distribution of ancestral states within a given tree that minimizes the total number of character state changes that would be necessary to explain the states observed at the tips of the tree. This method of maximum parsimony) is one of the earliest formalized algorithms for reconstructing ancestral states. Maximum parsimony can be implemented by one of several algorithms. One of the earliest examples is Fitch's method (Fitch WM. Toward defining the course of evolution: minimum change for a specific tree topology. Systematic Biology. 1971; 20 (4): 406-16), which assigns ancestral character states by parsimony via two traversals of a rooted binary tree. The first stage is a post-order traversal that proceeds from the tips toward the root of a tree by visiting descendant (child) nodes before their parents. Initially, the set of possible character states are determined, Sᵢ for the i-th ancestor based on the observed character states of its descendants. Each assignment is the set intersection of the character states of the ancestor's descendants; if the intersection is the empty set, then it is the set union. In the latter case, it is implied that a character state change has occurred between the ancestor and one of its two immediate descendants. Each such event counts towards the algorithm's cost function, which may be used to discriminate among alternative trees on the basis of maximum parsimony. Next, a preorder traversal of the tree is performed, proceeding from the root towards the tips. Character states are then assigned to each descendant based on which character states it shares with its parent. Since the root has no parent node, one may be required to select a character state arbitrarily, specifically when more than one possible state has been reconstructed at the root. Parsimony methods are intuitively appealing and highly efficient, such that they are still used in some cases to seed ML optimization algorithms with an initial phylogeny (Stamatakis A. RAXML-VI-HPC: maximum likelihood-based phylogenetic analyses with thousands of taxa and mixed models. Bioinformatics. 2006; 22:2688-90. pmid: 16928733). However, they suffer from several issues:
1. Variation in rates of evolution. Fitch's method assumes that changes between all character states are equally likely to occur; thus, any change incurs the same cost for a given tree. This assumption is often unrealistic and can limit the accuracy of such methods. For example, transitions tend to occur more often than transversions in the evolution of nucleic acids. This assumption can be relaxed by assigning differential costs to specific character state changes, resulting in a weighted parsimony algorithm (Sankoff D. Minimal mutation trees of sequences. SIAM Journal on Applied Mathematics. 1975; 28 (1): 35-42).
2. Rapid evolution. The upshot of the “minimum evolution” heuristic underlying such methods is that such methods assume that changes are rare and thus are inappropriate in cases where change is the norm rather than the exception (Schluter D, Price T, Mooers A O, Ludwig D. Likelihood of ancestor states in adaptive radiation. Evolution. 1997; 51 (6): 1699-711; Felsenstein J. Maximum likelihood and minimum-steps methods for estimating evolutionary trees from data on discrete characters. Systematic Biology. 1973; 22 (3): 240-9).
3. Variation in time among lineages. Parsimony methods implicitly assume that the same amount of evolutionary time has passed along every branch of the tree. Thus, they do not account for variation in branch lengths in the tree, which are often used to quantify the passage of evolutionary or chronological time. This limitation makes the technique liable to infer that one change occurred on a very short branch rather than multiple changes occurring on a very long branch, for example. This shortcoming is addressed by model-based methods (both ML and Bayesian methods) that infer the stochastic process of evolution as it unfolds along each branch of a tree (Li G, Steel M, Zhang L. More taxa are not necessarily better for the reconstruction of ancestral character states. Systematic biology. 2008; 57 (4): 647-53).
4. Statistical justification. Without a statistical model underlying the method, its estimates do not have well-defined uncertainties.

### Maximum Likelihood (ML)

ML methods of ancestral sequence reconstruction treat the character states at internal nodes of the tree as parameters and attempt to find the parameter values that maximize the probability of the data (the observed character states) given the hypothesis (a model of evolution and a phylogeny relating the observed sequences or taxa). Some of the earliest ML approaches to ancestral reconstruction were developed in the context of genetic sequence evolution (Yang Z, Kumar S, Nei M. A new method of inference of ancestral nucleotide and amino acid sequences. Genetics. 1995; 141 (4): 1641-50; Koshi J M, Goldstein R A. Probabilistic reconstruction of ancestral protein sequences. Journal of Molecular Evolution. 1996; 42 (2): 313-20); similar models were also developed for the analogous case of discrete character evolution (Pagel M. The maximum likelihood approach to reconstructing ancestral character states of discrete characters on phylogenies. Systematic biology. 1999; 48 (3): 612-22).

These approaches employ the same probabilistic framework as used to infer the phylogenetic tree (Felsenstein J. Evolutionary trees from DNA sequences: a maximum likelihood approach. Journal of molecular evolution. 1981; 17 (6): 368-76). In brief, the evolution of a genetic sequence is modelled by a time-reversible continuous time Markov process. In the simplest of these, all characters undergo independent state transitions (such as nucleotide substitutions) at a constant rate over time. This basic model is frequently extended to allow different rates on each branch of the tree. In reality, mutation rates may also vary over time (due, for example, to environmental changes); this can be modelled by allowing the rate parameters to evolve along the tree, at the expense of having an increased number of parameters. A model defines transition probabilities from states i to j along a branch of length t (in units of evolutionary time). The likelihood of a phylogeny is computed from a nested sum of transition probabilities that corresponds to the hierarchical structure of the proposed tree. At each node, the likelihood of its descendants is summed over all possible ancestral character states at that node:

$${L}_{\text{?}} = {\sum }_{\text{?}} P ( {S}_{\text{?}} ) ( {\sum }_{\text{?}} P ( {S}_{\text{?}} \vert {S}_{\text{?}} , {t}_{\text{?}} ) {L}_{\text{?}} ) {\sum }_{\text{?}} P ( {S}_{\text{?}} \vert {S}_{\text{?}} , {t}_{\text{?}} ) {L}_{\text{?}} )$$

$$\text{?} \text{indicates text missing or illegible when filed}$$

where the likelihood of the subtree rooted at node x with direct descendants y and z is computed, Sᵢ denotes the character state of the i-th node, tᵢⱼ is the branch length (evolutionary time) between nodes i and j, and Ω is the set of all possible character states (for example, the nucleotides A, C, G, and T). Thus, the objective of ancestral reconstruction is to find the assignment to Sₓ for all x internal nodes that maximizes the likelihood of the observed data for a given tree.

Rather than compute the overall likelihood for alternative trees, the problem for ancestral reconstruction is to find the combination of character states at each ancestral node with the highest marginal ML. Generally speaking, there are two approaches to this problem. First, one may work upwards from the descendants of a tree to progressively assign the most likely character state to each ancestor taking into consideration only its immediate descendants. This approach is referred to as marginal reconstruction. It is akin to a greedy algorithm that makes the locally optimal choice at each stage of the optimization problem. While it can be highly efficient, it is not guaranteed to attain a globally optimal solution to the problem. Second, one may instead attempt to find the joint combination of ancestral character states throughout the tree that jointly maximizes the likelihood of the data. Thus, this approach is referred to as joint reconstruction. While it is not as rapid as marginal reconstruction, it is also less likely to be caught in the local optima in nonconvex objective functions that modern optimization methods and heuristics are designed to avoid. In the context of ancestral reconstruction, this means that a marginal reconstruction may assign a character state to the immediate ancestor that is locally optimal but deflects the joint distribution of ancestral character states away from the global optimum. Joint reconstruction is more computationally complex than marginal reconstruction. Nevertheless, efficient algorithms for joint reconstruction have been developed with a time complexity that is generally linear with the number of observed taxa or sequences.

ML-based methods of ancestral reconstruction tend to provide greater accuracy than MP methods in the presence of variation in rates of evolution among characters (or across sites in a genome). However, these methods are not yet able to accommodate variation in rates of evolution over time, otherwise known as heterotachy. If the rate of evolution for a specific character accelerates on a branch of the phylogeny, then the amount of evolution that has occurred on that branch will be underestimated for a given length of the branch and assuming a constant rate of evolution for that character. In addition to that, it is difficult to distinguish heterotachy from variation among characters in rates of evolution.

Since ML (unlike maximum parsimony) requires the investigator to specify a model of evolution, its accuracy may be affected by the use of a grossly incorrect model (model misspecification). Furthermore, ML can only provide a single reconstruction of character states (what is often referred to as a “point estimate”)—when the likelihood surface is highly nonconvex, comprising multiple peaks (local optima), then a single point estimate cannot provide an adequate representation, and a Bayesian approach may be more suitable.

### Bayesian Inference

Bayesian inference uses the likelihood of observed data to update the investigator's belief, or prior distribution, to yield the posterior distribution. In the context of ancestral reconstruction, the objective is to infer the posterior probabilities of ancestral character states at each internal node of a given tree. Moreover, one can integrate these probabilities over the posterior distributions over the parameters of the evolutionary model and the space of all possible trees. This can be expressed as an application of Bayes' theorem:

$$P ( S \vert D , θ ) = \frac{P ( D \vert S , θ ) P ( S \vert θ )}{P ( D \vert θ )} ∝ P ( S \vert D , θ ) ( S \vert θ ) P ( θ ) ,$$

where S represents the ancestral states, D corresponds to the observed data, and θ represents both the evolutionary model and the phylogenetic tree. P(D|S,θ) is the likelihood of the observed data that can be computed by Felsenstein's pruning algorithm as given above. P(S|θ) is the prior probability of the ancestral states for a given model and tree. Finally, P(D|θ) is the probability of the data for a given model and tree, integrated over all possible ancestral states. Two formulations are given to emphasize the two different applications of Bayes' theorem, discussed below.

One of the first implementations of a Bayesian approach to ancestral sequence reconstruction was developed by Yang and colleagues, where the ML estimates of the evolutionary model and tree, respectively, were used to define the prior distributions. Thus, their approach is an example of an empirical Bayes method to compute the posterior probabilities of ancestral character states; this method was first implemented in the software package PAML (Yang Z. PAML 4: phylogenetic analysis by maximum likelihood. Molecular biology and evolution. 2007; 24 (8): 1586-91). In terms of the above Bayesian rule formulation, the empirical Bayes method fixes to the empirical estimates of the model and tree obtained from the data, effectively dropping from the posterior likelihood and prior terms of the formula. Moreover, Yang and colleagues (Yang Z, Kumar S, Nei M. A new method of inference of ancestral nucleotide and amino acid sequences. Genetics. 1995; 141 (4): 1641-50) used the empirical distribution of site patterns (i.e., assignments of nucleotides to tips of the tree) in their alignment of observed nucleotide sequences in the denominator in place of exhaustively computing P(D) over all possible values of S, given θ. Computationally, the empirical Bayes method is akin to the ML reconstruction of ancestral states except that, rather than searching for the ML assignment of states based on their respective probability distributions at each internal node, the probability distributions themselves are reported directly.

Empirical Bayes methods for ancestral reconstruction require the investigator to assume that the evolutionary model parameters and tree are known without error. When the size or complexity of the data makes this an unrealistic assumption, it may be more prudent to adopt the fully hierarchical Bayesian approach and infer the joint posterior distribution over the ancestral character states, model, and tree (Huelsenbeck J P, Bollback J P. Empirical and hierarchical Bayesian estimation of ancestral states. Systematic Biology. 2001; 50 (3): 351-66). Huelsenbeck and Bollback first proposed a hierarchical Bayes method to ancestral reconstruction by using Markov chain Monte Carlo (MCMC) methods to sample ancestral sequences from this joint posterior distribution. A similar approach was also used to reconstruct the evolution of symbiosis with algae in fungal species (lichenization) (Lutzoni F, Pagel M, Reeb V. Major fungal lineages are derived from lichen symbiotic ancestors. Nature. 2001; 411 (6840): 937-40). For example, the Metropolis-Hastings algorithm for MCMC explores the joint posterior distribution by accepting or rejecting parameter assignments on the basis of the ratio of posterior probabilities.

Thus, the empirical Bayes approach calculates the probabilities of various ancestral states for a specific tree and model of evolution. By expressing the reconstruction of ancestral states as a set of probabilities, one can directly quantify the uncertainty for assigning any particular state to an ancestor. On the other hand, the hierarchical Bayes approach averages these probabilities over all possible trees and models of evolution, in proportion to how likely these trees and models are, given the data that has been observed.

The fully Bayesian approach is limited to analyzing relatively small numbers of sequences or taxa because the space of all possible trees rapidly becomes too vast, making it computationally infeasible for chain samples to converge in a reasonable amount of time.

Pathogens, especially emerging or re-emerging pathogens, such as emerging or re-emerging RNA viruses, evolve at an extremely rapid rate, orders of magnitude faster than mammals or birds. For these organisms, ancestral reconstruction can be applied on a much shorter time scale, for example, to reconstruct the global or regional progenitor of an epidemic that has spanned decades rather than millions of years. It has been proposed that such reconstructed strains be used as targets for vaccine design efforts as opposed to sequences isolated from patients in the present day (Gaschen et al., Science. 2002; 296 (5577): 2354-60).

According to embodiments of methods of the invention, any suitable method of ARS may be used to identify amino acid sequence or encoded amino acid sequence that is ancestral amino acid sequence from the multiple sequence alignment.

Optionally identification of ancestral amino acid sequence from the multiple sequence alignment comprises performing a maximum parsimony ancestral sequence reconstruction (MP-ASR).

Optionally identification of ancestral amino acid sequence from the multiple sequence alignment comprises performing a maximum likelihood ancestral sequence reconstruction (ML-ASR).

Optionally identification of ancestral amino acid sequence from the multiple sequence alignment comprises performing a Bayesian inference ancestral sequence reconstruction (BI-ASR).

There are many software packages available that perform ancestral sequence reconstruction. The following table (taken from Joy et al., 2016, PLOS Computational Biology 12 (7): DOI: 10.1371/journal.pcbi. 1004763) provides a representative sample of the extensive variety of packages that implement methods of ancestral reconstruction with different strengths and features:

| | | | | | Continuous (C) or | |
|---|---|---|---|---|---|---|
| | | | | | Discrete (D) | |
| Name | Methods | Platform | Supported Input [symbol] | Character Types | Characters | Software [symbol] |
| PAML | ML | [symbol] | [symbol] | [symbol] | D | [symbol] |
| BEAST2 | [symbol] | [symbol] | [symbol] | [symbol] | C, D | [symbol] |
| APE | ML | [symbol] | [symbol] | [symbol] | C, D | [symbol] |
| [symbol] | ML | [symbol] | [symbol] | [symbol] | C, D | [symbol] |
| [symbol] | ML | [symbol] | [symbol] | [symbol] | D | [symbol] |
| [symbol] | [symbol] | [symbol] | [symbol] | [symbol] | C, D | [symbol] |
| [symbol] | ML | [symbol] | [symbol] | [symbol] | — | [symbol] |
| [symbol] | [symbol] | [symbol] | [symbol] | [symbol] | C, D | [symbol] |
| [symbol] | [symbol] | [symbol] | NEXUS | [symbol] | C, D | — |
| [symbol] | ML | [symbol] | [symbol] | [symbol] | D | — |
| [symbol] | [symbol] | [symbol] | [symbol] | [symbol] | C, D | [symbol] |
| [symbol] | [symbol] | [symbol] | [symbol] | [symbol] | D | [symbol] |
| [symbol] | [symbol] | [symbol] | NEXUS | [symbol] | D | [symbol] |
| [symbol] | [symbol] | [symbol] | [symbol] | [symbol] | D | [symbol] |
| [symbol] | ML | [symbol] | [symbol] | [symbol] | D | [symbol] |
| [symbol] | [symbol] | [symbol] | [symbol] | [symbol] | D | — |
| VIP | [symbol] | [symbol] | [symbol] | [symbol] | O [symbol] | [symbol] |
| [symbol] | ML | [symbol] | [symbol] | [symbol] | D | [symbol] |
| [symbol] | ML | [symbol] | [symbol] | [symbol] | D | [symbol] |
| [symbol] | [symbol] | [symbol] | [symbol] | [symbol] | D | [symbol] |
| COUNT | [symbol] | [symbol] | [symbol] | [symbol] | D | BSD |
| MEGA | [symbol] | [symbol] | [symbol] | [symbol] | D | [symbol] |
| ANGES | [symbol] | [symbol] | [symbol] | [symbol] | D | [symbol] |
| EREM | ML | [symbol] | [symbol] | [symbol] | D | [symbol] |

[symbol]

[symbol]

indicates data missing or illegible when filed

The majority of these software packages are designed for analyzing genetic sequence data. For example, PAML (Yang Z. PAML 4: phylogenetic analysis by maximum likelihood. Molecular biology and evolution. 2007; 24 (8): 1586-91) is a collection of programs for the phylogenetic analysis of DNA and protein sequence alignments by ML. Ancestral reconstruction can be performed using the codeml program. HyPhy, Mesquite, and MEGA are also software packages for the phylogenetic analysis of sequence data, but are designed to be more modular and customizable. HyPhy (Pond S L K, Muse S V. HyPhy: hypothesis testing using phylogenies. Statistical methods in molecular evolution: Springer; 2005. p. 125-81) implements a joint ML method of ancestral sequence reconstruction (Pupko T, Pe I, Shamir R, Graur D. A fast algorithm for joint reconstruction of ancestral amino acid sequences. Molecular Biology and Evolution. 2000; 17 (6): 890-6) that can be readily adapted to reconstructing a more generalized range of discrete ancestral character states such as geographic locations by specifying a customized model in its batch language. Mesquite (Maddison W, Maddison D. Mesquite: a modular system for evolutionary analysis. 2.75 ed20011) provides ancestral state reconstruction methods for both discrete and continuous characters using both maximum parsimony and ML methods. It also provides several visualization tools for interpreting the results of ancestral reconstruction. MEGA (Tamura K, Dudley J, Nei M, Kumar S. MEGA4: molecular evolutionary genetics analysis (MEGA) software version 4.0. Molecular biology and evolution. 2007; 24 (8): 1596-9) is a modular system, too, but places greater emphasis on ease-of-use than customization of analyses. As of version 5, MEGA allows the user to reconstruct ancestral states using maximum parsimony, ML, and empirical Bayes methods.

The Bayesian analysis of genetic sequences may confer greater robustness to model misspecification. MrBayes (Huelsenbeck J P, Ronquist F. MRBAYES: Bayesian inference of phylogenetic trees. Bioinformatics. 2001; 17 (8): 754-5) allows inference of ancestral states at ancestral nodes using the full hierarchical Bayesian approach. The PREQUEL program distributed in the PHAST package performs comparative evolutionary genomics using ancestral sequence reconstruction (Hubisz M J, Pollard K S, Siepel A. PHAST and RPHAST: phylogenetic analysis with space/time models. Briefings in bioinformatics. 2011; 12 (1): 41-51). SIMMAP stochastically maps mutations on phylogenies (Bollback J P. SIMMAP: stochastic character mapping of discrete traits on phylogenies. BMC bioinformatics. 2006; 7 (1): 88). Bayes Traits (Pagel M. The maximum likelihood approach to reconstructing ancestral character states of discrete characters on phylogenies. Systematic biology. 1999; 48 (3): 612-22) analyses discrete or continuous characters in a Bayesian framework to evaluate models of evolution, reconstruct ancestral states, and detect correlated evolution between pairs of traits.

Other software packages are more oriented towards the analysis of qualitative and quantitative traits (phenotypes). For example, the ape package (Paradis E. Analysis of phylogenetics and evolution with R. New York: Springer; 2006) in the statistical computing environment R also provides methods for ancestral state reconstruction for both discrete and continuous characters through the ace function, including ML. Note that ace performs reconstruction by computing scaled conditional likelihoods instead of the marginal or joint likelihoods used by other ML-based methods for ancestral reconstruction, which may adversely affect the accuracy of reconstruction at nodes other than the root. Phyrex implements a maximum parsimony-based algorithm to reconstruct ancestral gene expression profiles in addition to a ML method for reconstructing ancestral genetic sequences (by wrapping around the baseml function in PAML) (Rossnes R, Eidhammer I, Liberles D A. Phylogenetic reconstruction of ancestral character states for gene expression and mRNA splicing data. BMC bioinformatics. 2005; 6 (1): 127).

Several software packages also reconstruct phylogeography. BEAST (Bayesian Evolutionary Analysis by Sampling Trees (Bouckaert R, Heled J, Kühnert D, Vaughan T, Wu C—H, Xie D, et al. BEAST 2: a software platform for Bayesian evolutionary analysis. PLOS Comput Biol. 2014; 10 (4): e1003537)) provides tools for reconstructing ancestral geographic locations from observed sequences annotated with location data using Bayesian MCMC sampling methods. Diversitree (FitzJohn RG. Diversitree: comparative phylogenetic analyses of diversification in R. Methods in Ecology and Evolution. 2012; 3 (6): 1084-92) is an R package providing methods for ancestral state reconstruction under Mk2 (a continuous time Markov model of binary character evolution (Pagel M. Detecting Correlated Evolution on Phylogenies—a General-Method for the Comparative-Analysis of Discrete Characters. Proceedings of the Royal Society of London Series B-Biological Sciences. 1994; 255 (1342): 37-45)) and BiSSE models. Lagrange performs analyses on reconstruction of geographic range evolution on phylogenetic trees (Ree R H, Smith S A. Maximum likelihood inference of geographic range evolution by dispersal, local extinction, and cladogenesis. Systematic Biology. 2008; 57 (1): 4-14). Phylomapper (Lemmon A R, Lemmon E M. A likelihood framework for estimating phylogeographic history on a continuous landscape. Systematic Biology. 2008; 57 (4): 544-61) is a statistical framework for estimating historical patterns of gene flow and ancestral geographic locations. RASP (Yu Y, Harris A J, Blair C, He X. RASP (Reconstruct Ancestral State in Phylogenies): a tool for historical biogeography. Molecular Phylogenetics and Evolution. 2015; 87:46-9) infers ancestral state using statistical DIVA, Lagrange, Bayes-Lagrange, BayArea, and BBM methods. VIP (Arias J S, Szumik C A, Goloboff P A. Spatial analysis of vicariance: a method for using direct geographical information in historical biogeography. Cladistics. 2011; 27 (6): 617-28) infers historical biogeography by examining disjunct geographic distributions.

Genome rearrangements provide valuable information in comparative genomics between species. ANGES (Jones B R, Rajaraman A, Tannier E, Chauve C. ANGES: reconstructing ANcestral GEnomeS maps. Bioinformatics. 2012; 28 (18): 2388-90) compares extant-related genomes through ancestral reconstruction of genetic markers. BADGER (Larget B, Kadane J B, Simon D L. A Bayesian approach to the estimation of ancestral genome arrangements. Molecular phylogenetics and evolution. 2005; 36 (2): 214-23) uses a Bayesian approach to examining the history of gene rearrangement. Count (Csuos M. Count: evolutionary analysis of phylogenetic profiles with parsimony and likelihood. Bioinformatics. 2010; 26 (15): 1910-2) reconstructs the evolution of the size of gene families. EREM (Affre L, Thompson J D, Debussche M. Genetic structure of continental and island populations of the Mediterranean endemic Cyclamen balearicum (Primulaceae). American Journal of Botany. 1997; 84 (4): 437-51) analyses the gain and loss of genetic features encoded by binary characters. PARANA (Patro R, Sefer E, Malin J, Marçais G, Navlakha S, Kingsford C. Parsimonious reconstruction of network evolution. Algorithms for Molecular Biology. 2012; 7 (1): 1) performs parsimony-based inference of ancestral biological networks that represent gene loss and duplication.

There are also several web server-based applications that allow investigators to use ML methods for ancestral reconstruction of different character types without having to install any software. For example, Ancestors (Diallo A B, Makarenkov V, Blanchette M. Ancestors 1.0: a web server for ancestral sequence reconstruction. Bioinformatics. 2010; 26 (1): 130-1) is a web server for ancestral genome reconstruction by the identification and arrangement of syntenic regions. FastML (Ashkenazy H, Penn O, Doron-Faigenboim A, Cohen O, Cannarozzi G, Zomer O, et al. FastML: a web server for probabilistic reconstruction of ancestral sequences. Nucleic acids research. 2012; 40 (W1): W580-W4) is a web server for probabilistic reconstruction of ancestral sequences by ML that uses a gap character model for reconstructing indel variation. MLGO (Hu F, Lin Y, Tang J. MLGO: phylogeny reconstruction and ancestral inference from gene-order data. BMC bioinformatics. 2014; 15 (1): 1) is a web server for ML gene order analysis.

A candidate optimized antigenic pathogen polypeptide of the polypeptide library may comprise one or more regions of amino acid sequence that have been identified through ARS. Optionally for a candidate optimized antigenic pathogen polypeptide the, or each region of ancestral amino acid sequence is at least 1, 2, 3, 5, 10, 15, 20, 25, 30, 35, 40, 45, or 50 amino acid residues long. Optionally for a candidate optimized antigenic pathogen polypeptide the, or each region of ancestral amino acid sequence is up to 5, 10, 15, 20, 25, 30, 40, 50, 100, 150, 200, 250, 300, 350,400, 450, or 500, 600, 700, or 800 amino acid residues long.

Optionally a candidate optimized antigenic pathogen polypeptide of the polypeptide library comprises an amino acid sequence that has at least 20%, 30%, 40%, 50%, 60%, 70%, 80%, 90%, 95%, 96%, 97%, 98%, or 99% amino acid identity along its entire length with an amino acid sequence of a pathogen polypeptide of one or more of the different isolates from which the candidate optimized antigenic pathogen polypeptide was optimized.

Optionally methods of the invention include optimizing codons of the different generated nucleotide sequences for optimal expression of the encoded candidate optimized antigenic pathogen polypeptides in an expression system. Codon optimization takes advantage of the degeneracy of the genetic code, and does not alter the amino acid sequence of the encoded polypeptide. Because of degeneracy, one protein can be encoded by many alternative nucleic acid sequences. Codon preference (codon usage bias) differs in each organism, and this can create challenges for expressing recombinant proteins in heterologous expression systems, resulting in low and unreliable expression.

Any suitable expression system may be used. Several suitable examples are well known to the skill person, including expression in a mammalian, yeast, insect, or bacterial cell. Optionally the expression system comprises a mammalian cell. Optionally the expression system comprises a yeast, an insect, or a bacterial cell.

Methods of codon-optimization are well known to those of ordinary skill in the art. A codon optimization algorithm may be used to design a codon-optimized nucleotide sequence encoding an amino acid sequence. Such algorithms are aimed at providing codon-optimized sequences which maximise expression of a polypeptide or protein in a desired expression system. Examples of suitable codon optimization algorithms include GeneOptimizer™ algorithm (ThermoFisher), OptimumGene™ algorithm (GenScript), and GeneGPS® (ATUM).

Optionally methods of the invention also include other sequence optimization to maximise protein expression in a desired expression system. Such gene optimization takes account of codon usage bias, as well as other sequence-related parameters involved in gene expression, such as transcription, splicing, translation, and mRNA degradation. Examples of such sequence-related parameters are given below (the parameters are classed below as affecting transcriptional efficiency, translational efficiency, or protein refolding, but several of the parameters may influence more than one of these steps):

### Transcriptional Efficacy:

| | |
|---|---|
| GC content | SD sequence |
| CpG dinucleotides content | TATA boxes |
| Cryptic splicing sites | Terminal signal |
| Negative CpG islands | Artificial recombination sites |

### Translational Efficiency:

| | |
|---|---|
| Codon usage bias | RNA instability motif (ARE) |
| GC content | Stable free energy of mRNA |
| mRNA secondary structure | Internal chi sites and ribosomal |
| | binding sites |
| Premature PolyA sites | Repetitive sequences |

### Protein Refolding:

| | |
|---|---|
| Codon usage bias | Codon-context |
| Interaction of codon and anti-codon | RNA secondary structures |

Gene optimization algorithms, such as GeneOptimizer™ and OptimumGene™, take account of several of these parameters.

Gene optimization for expression of human proteins in E. coli is discussed by Maertens et al. (Protein Science 2010 Vol. 19:1312-1326).

Optionally methods of the invention include optimizing the different nucleotide sequences for antigenicity of the encoded candidate optimized antigenic pathogen polypeptides.

Antigenic optimization may include any of the following:
(a) deletion or modification of nucleic acid sequence encoding amino acid sequence believed to inhibit production and/or function of anti-pathogen polypeptide antibody (for example, deletion or modification of a mucin-like domain-see Reynard et al., Journal of Virology, 2009, 9596-9601);
(b) region swapping to recover one or more potential lost encoded epitopes;
(c) site-specific mutation, for example of N-linked glycosylation sites. Typically site-specific mutation is designed to delete N-linked glycosylation sites, although there may be situations where additional sites might be desired to be introduced, for instance to mask epitopes that elicit non-neutralizing antibodies. The ability of glycosylation to sterically block antibody binding to HA and thus provide protection against the host immune response has been demonstrated for influenza viruses. Sun et al. (Journal of Virology, 2013, 87 (15): 8756-8766) demonstrate that antibodies induced by viruses with a high number of glycosylation sites have a broader neutralizing activity than the antibodies induced by the viruses with fewer glycosylation sites;
(d) changes to enhance stability (e.g. disulphide bond formation, reduce degradation of the encoded polypeptide by a serine protease);
(e) removal of glycans (improve access for B-cells);
(f) insertion of nucleic acid sequence, for example to insert nucleic acid sequence encoding a desired epitope.

Antigenic optimization of the outer domain of HIV-1 gp120 is described by Joyce et al. (J Virol. 2013 February; 87 (4): 2294-306).

Optionally the different pathogen isolates include different pathogen isolates from an outbreak of a pathogen of the same subtype as the pathogen to which it is desired to induce a broadly neutralizing immune response.

Optionally the different pathogen isolates include different pathogen isolates from an outbreak of a pathogen of a different subtype, but the same type, as the pathogen to which it is desired to induce a broadly neutralizing immune response.

Optionally the different pathogen isolates include different pathogen isolates from an outbreak of a pathogen of a different type, but the same family, as the pathogen to which it is desired to induce a broadly neutralizing immune response.

Optionally the different pathogen isolates include different prior pathogen isolates of a pathogen of the same subtype, type, or family as the pathogen to which it is desired to induce a broadly neutralizing immune response.

Optionally the different pathogen isolates include different prior pathogen isolates of a pathogen of the same species, genera, or family as the pathogen to which it is desired to induce a broadly neutralizing immune response.

Optionally methods of the invention for identifying a lead candidate optimized antigenic pathogen polypeptide capable of inducing a broadly neutralizing immune response to a pathogen are in vitro methods.

According to the invention there is also provided a method of identifying a nucleic acid sequence encoding an optimized antigenic pathogen polypeptide capable of inducing a broadly neutralizing immune response to a pathogen, which comprises:
i) immunizing a human, or a non-human animal, with a nucleic acid comprising a nucleic acid sequence encoding a lead candidate optimized antigenic pathogen polypeptide identified by a method according to the invention;
ii) determining whether a broadly neutralizing immune response is induced in the human or non-human animal following the immunization in step (i); and
iii) identifying the nucleic acid sequence as a nucleic acid sequence encoding an optimized antigenic pathogen polypeptide capable of inducing a broadly neutralizing immune response to the pathogen if it is determined from step (ii) that a broadly neutralizing immune response is induced in the human or non-human animal.

Optionally it is determined whether a broadly neutralizing immune response is induced in the human or non-human animal by determining whether antibody in serum obtained from the human or non-human animal binds to more than one pathogen subtype within the same family as the pathogen to which a broadly neutralizing immune response is desired.

Optionally it is determined whether a broadly neutralizing immune response is induced in the human or non-human animal by determining whether antibody in serum obtained from the human or non-human animal binds to more than one pathogen type within the same family as the pathogen to which a broadly neutralizing immune response is desired.

Any suitable non-human animal may be used. Optionally the non-human animal is a mammal. Optionally the mammal is a guinea pig, or a mouse. Optionally the non-human animal is avian.

According to the invention there is also provided an isolated nucleic acid molecule, comprising a nucleic acid sequence that is:
i) at least 75%, 80%, 85%, 90%, 95%, 96%, 97%, 98%, or 99% identical with SEQ ID NO: 1, or identical with SEQ ID NO:1;
ii) at least 75%, 80%, 85%, 90%, 95%, 96%, 97%, 98%, or 99% identical with SEQ ID NO: 2, or identical with SEQ ID NO:2;
iii) at least 75%, 80%, 85%, 90%, 95%, 96%, 97%, 98%, or 99% identical with SEQ ID NO: 4, or identical with SEQ ID NO:4;
iv) at least 75%, 80%, 85%, 90%, 95%, 96%, 97%, 98%, or 99% identical with SEQ ID NO: 5, or identical with SEQ ID NO:5;
v) at least 75%, 80%, 85%, 90%, 95%, 96%, 97%, 98%, or 99% identical with SEQ ID NO: 7, or identical with SEQ ID NO:7; or
vi) at least 75%, 80%, 85%, 90%, 95%, 96%, 97%, 98%, or 99% identical with SEQ ID NO: 8, or identical with SEQ ID NO:8;
   or the complement thereof.

There is also provided according to the invention an isolated nucleic acid molecule, comprising a nucleic acid sequence that is:
i) at least 75%, 80%, 85%, 90%, 95%, 96%, 97%, 98%, or 99% identical with SEQ ID NO: 10, or identical with SEQ ID NO: 10;
ii) at least 75%, 80%, 85%, 90%, 95%, 96%, 97%, 98%, or 99% identical with SEQ ID NO: 12, or identical with SEQ ID NO: 12; or
iii) at least 75%, 80%, 85%, 90%, 95%, 96%, 97%, 98%, or 99% identical with SEQ ID NO: 14, or identical with SEQ ID NO: 14;
or the complement thereof.

There is also provided according to the invention an isolated nucleic acid molecule, comprising a nucleic acid sequence that is:
i) at least 75%, 80%, 85%, 90%, 95%, 96%, 97%, 98%, or 99% identical with SEQ ID NO: 19, or identical with SEQ ID NO: 19;
ii) at least 75%, 80%, 85%, 90%, 95%, 96%, 97%, 98%, or 99% identical with SEQ ID NO: 21, or identical with SEQ ID NO:21;
iii) at least 75%, 80%, 85%, 90%, 95%, 96%, 97%, 98%, or 99% identical with SEQ ID NO: 23, or identical with SEQ ID NO:23;
iv) at least 75%, 80%, 85%, 90%, 95%, 96%, 97%, 98%, or 99% identical with SEQ ID NO: 25, or identical with SEQ ID NO:25;
v) at least 75%, 80%, 85%, 90%, 95%, 96%, 97%, 98%, or 99% identical with SEQ ID NO: 27, or identical with SEQ ID NO:27;
vi) at least 75%, 80%, 85%, 90%, 95%, 96%, 97%, 98%, or 99% identical with SEQ ID NO: 29, or identical with SEQ ID NO:29; or
vii) at least 75%, 80%, 85%, 90%, 95%, 96%, 97%, 98%, or 99% identical with SEQ ID NO: 31, or identical with SEQ ID NO:31;
or the complement thereof.

According to the invention there is further provided an isolated polypeptide, comprising an amino acid sequence that is:
i) at least 95%, 96%, 97%, 98%, or 99% identical with an amino acid sequence encoded by SEQ ID NO: 1, or identical with the amino acid sequence encoded by SEQ ID NO: 1;
ii) at least 95%, 96%, 97%, 98%, or 99% identical with an amino acid sequence encoded by SEQ ID NO:2, or identical with the amino acid sequence encoded by SEQ ID NO: 2;
iii) at least 95%, 96%, 97%, 98%, or 99% identical with an amino acid sequence encoded by SEQ ID NO:4, or identical with the amino acid sequence encoded by SEQ ID NO: 4;
iv) at least 95%, 96%, 97%, 98%, or 99% identical with an amino acid sequence encoded by SEQ ID NO:5, or identical with the amino acid sequence encoded by SEQ ID NO: 5;
v) at least 95%, 96%, 97%, 98%, or 99% identical with an amino acid sequence encoded by SEQ ID NO:7, or identical with the amino acid sequence encoded by SEQ ID NO: 7;
vi) at least 95%, 96%, 97%, 98%, or 99% identical with an amino acid sequence encoded by SEQ ID NO:8, or identical with the amino acid sequence encoded by SEQ ID NO: 8;
vii) at least 95%, 96%, 97%, 98%, or 99% identical with an amino acid sequence encoded by SEQ ID NO: 10, or identical with the amino acid sequence encoded by SEQ ID NO: 10;
viii) at least 95%, 96%, 97%, 98%, or 99% identical with an amino acid sequence encoded by SEQ ID NO: 12, or identical with the amino acid sequence encoded by SEQ ID NO: 12; or
ix) at least 95%, 96%, 97%, 98%, or 99% identical with an amino acid sequence encoded by SEQ ID NO: 14, or identical with the amino acid sequence encoded by SEQ ID NO: 14.

There is also provided according to the invention an isolated polypeptide, comprising an amino acid sequence that is:
i) at least 95%, 96%, 97%, 98%, or 99% identical with SEQ ID NO:3, or identical with SEQ ID NO:3;
ii) at least 95%, 96%, 97%, 98%, or 99% identical with SEQ ID NO:6, or identical with SEQ ID NO:6;
iii) at least 95%, 96%, 97%, 98%, or 99% identical with SEQ ID NO:9, or identical with SEQ ID NO:9;
iv) at least 95%, 96%, 97%, 98%, or 99% identical with SEQ ID NO: 11, or identical with SEQ ID NO:11;
v) at least 95%, 96%, 97%, 98%, or 99% identical with SEQ ID NO: 13, or identical with SEQ ID NO:13; or
vi) at least 95%, 96%, 97%, 98%, or 99% identical with SEQ ID NO: 15, or identical with SEQ ID NO:15.

There is also provided according to the invention an isolated polypeptide, comprising an amino acid sequence that is:
i) at least 95%, 96%, 97%, 98%, or 99% identical with SEQ ID NO: 18, or identical with SEQ ID NO:18;
ii) at least 95%, 96%, 97%, 98%, or 99% identical with SEQ ID NO:20, or identical with SEQ ID NO:20;
iii) at least 95%, 96%, 97%, 98%, or 99% identical with SEQ ID NO:22, or identical with SEQ ID NO:22;
iv) at least 95%, 96%, 97%, 98%, or 99% identical with SEQ ID NO:24, or identical with SEQ ID NO:24;
v) at least 95%, 96%, 97%, 98%, or 99% identical with SEQ ID NO:26, or identical with SEQ ID NO:26;
vi) at least 95%, 96%, 97%, 98%, or 99% identical with SEQ ID NO:28, or identical with SEQ ID NO:28; or
vii) at least 95%, 96%, 97%, 98%, or 99% identical with SEQ ID NO:30, or identical with SEQ ID NO:30.

The similarity between amino acid or nucleic acid sequences is expressed in terms of the similarity between the sequences, otherwise referred to as sequence identity.

Sequence identity is frequently measured in terms of percentage identity (or similarity or homology); the higher the percentage, the more similar the two sequences are. Homologs or variants of a given gene or protein will possess a relatively high degree of sequence identity when aligned using standard methods. Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237-244, 1988; Higgins and Sharp, CABIOS 5:151-153, 1989; Corpet et al., Nucleic Acids' Research 16:10881-10890, 1988; and Pearson and Lipman, Proc. Natl. Acad. Sci. U.S.A. 85:2444, 1988. Altschul et al., Nature Genet. 6:119-129, 1994. The NCBI Basic Local Alignment Search Tool (BLAST™) (Altschul et al., J. Mol. Biol. 215:403-410, 1990) is available from several sources, including the National Center for Biotechnology Information (NCBI, Bethesda, MD) and on the Internet, for use in connection with the sequence analysis programs blastp, blastn, blastx, tblastn and tblastx.

Sequence identity between nucleic acid sequences, or between amino acid sequences, can be determined by comparing an alignment of the sequences. When an equivalent position in the compared sequences is occupied by the same nucleotide, or amino acid, then the molecules are identical at that position. Scoring an alignment as a percentage of identity is a function of the number of identical nucleotides or amino acids at positions shared by the compared sequences. When comparing sequences, optimal alignments may require gaps to be introduced into one or more of the sequences to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties.

Suitable computer programs for carrying out sequence comparisons are widely available in the commercial and public sector. Examples include MatGat (Campanella et al., 2003, BMC Bioinformatics 4:29; program available from http://bitincka.com/ledion/matgat), Gap (Needleman & Wunsch, 1970, J. Mol. Biol. 48:443-453), FASTA (Altschul et al., 1990, J. Mol. Biol. 215:403-410; program available from http://www.ebi.ac.uk/fasta), Clustal W 2.0 and X 2.0 (Larkin et al., 2007, Bioinformatics 23:2947-2948; program available from http://www.ebi.ac.uk/tools/clustalw2) and EMBOSS Pairwise Alignment Algorithms (Needleman & Wunsch, 1970, supra; Kruskal, 1983, In: Time warps, string edits and macromolecules: the theory and practice of sequence comparison, Sankoff & Kruskal (eds), pp 1-44, Addison Wesley; programs available from http://www.ebi.ac.uk/tools/emboss/align). All programs may be run using default parameters.

For example, sequence comparisons may be undertaken using the “needle” method of the EMBOSS Pairwise Alignment Algorithms, which determines an optimum alignment (including gaps) of two sequences when considered over their entire length and provides a percentage identity score. Default parameters for amino acid sequence comparisons (“Protein Molecule” option) may be Gap Extend penalty: 0.5, Gap Open penalty: 10.0, Matrix: Blosum 62.

The sequence comparison may be performed over the full length of the reference sequence. There is also provided according to the invention an isolated nucleic acid molecule which comprises a nucleotide sequence encoding a polypeptide comprising an amino acid sequence of SEQ ID NO: 6, and a polypeptide comprising an amino acid sequence of SEQ ID NO: 9.

There is also provided according to the invention an isolated nucleic acid molecule which comprises a nucleotide sequence encoding a polypeptide comprising an amino acid sequence of SEQ ID NO: 13, and a polypeptide comprising an amino acid sequence of SEQ ID NO: 15.

There is also provided according to the invention a composition comprising a first nucleic acid which includes a nucleotide sequence encoding a polypeptide comprising an amino acid sequence of SEQ ID NO: 6, and a second nucleic acid which includes a nucleotide sequence encoding a polypeptide comprising an amino acid sequence of SEQ ID NO: 9.

There is also provided according to the invention a composition comprising a first nucleic acid which includes a nucleotide sequence encoding a polypeptide comprising an amino acid sequence of SEQ ID NO: 13, and a second nucleic acid which includes a nucleotide sequence encoding a polypeptide comprising an amino acid sequence of SEQ ID NO: 15.

There is also provided according to the invention a combined preparation comprising: (i) a first nucleic acid which includes a nucleotide sequence encoding a polypeptide comprising an amino acid sequence of SEQ ID NO: 6; and (ii) a second nucleic acid which includes a nucleotide sequence encoding a polypeptide comprising an amino acid sequence of SEQ ID NO: 9.

There is also provided according to the invention a combined preparation comprising: (i) a first nucleic acid which includes a nucleotide sequence encoding a polypeptide comprising an amino acid sequence of SEQ ID NO: 13; and (ii) a second nucleic acid which includes a nucleotide sequence encoding a polypeptide comprising an amino acid sequence of SEQ ID NO: 15.

There is also provided according to the invention a composition comprising a first polypeptide comprising an amino acid sequence of SEQ ID NO: 6, and a second polypeptide comprising an amino acid sequence of SEQ ID NO: 9.

There is also provided according to the invention a composition comprising a first polypeptide comprising an amino acid sequence of SEQ ID NO: 13, and a second polypeptide comprising an amino acid sequence of SEQ ID NO: 15.

There is also provided according to the invention a fusion protein comprising a first polypeptide comprising an amino acid sequence of SEQ ID NO: 6, and a second polypeptide comprising an amino acid sequence of SEQ ID NO: 9.

There is also provided according to the invention a fusion protein comprising a first polypeptide comprising an amino acid sequence of SEQ ID NO: 13, and a second polypeptide comprising an amino acid sequence of SEQ ID NO: 15.

There is also provided according to the invention a combined preparation comprising: (i) a first polypeptide comprising an amino acid sequence of SEQ ID NO: 6; and (ii) a second polypeptide comprising an amino acid sequence of SEQ ID NO: 9.

There is also provided according to the invention a combined preparation comprising: (i) a first polypeptide comprising an amino acid sequence of SEQ ID NO: 13; and (ii) a second polypeptide comprising an amino acid sequence of SEQ ID NO: 15.

The term “combined preparation” as used herein refers to a “kit of parts” in the sense that the combination components (i) and (ii) as defined above can be dosed independently or by use of different fixed combinations with distinguished amounts of the combination components (i) and (ii). The components can be administered simultaneously or one after the other. If the components are administered one after the other, preferably the time interval between administration is chosen such that the therapeutic effect of the combined use of the components is greater than the effect which would be obtained by use of only any one of the combination components (i) and (ii).

The components of the combined preparation may be present in one combined unit dosage form, or as a first unit dosage form of component (i) and a separate, second unit dosage form of component (ii). The ratio of the total amounts of the combination component (i) to the combination component (ii) to be administered in the combined preparation can be varied, for example in order to cope with the needs of a patient sub-population to be treated, or the needs of the single patient, which can be due, for example, to the particular disease, age, sex, or body weight of the patient.

Preferably, there is at least one beneficial effect, for example an enhancing of the effect of component (i), or component (ii), or a mutual enhancing of the effect of the combination components (i) and (ii), for example a more than additive effect, additional advantageous effects, fewer side effects, less toxicity, or a combined therapeutic effect compared with an effective dosage of one or both of the combination components (i) and (ii), and very preferably a synergism of the combination components (i) and (ii).

A combined preparation of the invention may be provided as a pharmaceutical combined preparation for administration to a mammal, preferably a human. Component (i) may optionally be provided together with a pharmaceutically acceptable carrier, excipient, or diluent, and/or component (ii) may optionally be provided together with a pharmaceutically acceptable carrier, excipient, or diluent.

There is further provided according to the invention an isolated nucleic acid molecule encoding an amino acid sequence encoded by a nucleic acid of the invention.

There is further provided according to the invention an isolated nucleic acid molecule encoding an amino acid sequence encoded by a nucleic acid of the invention, wherein the nucleic acid is codon-optimized for expression in mammalian cells.

There is further provided according to the invention an isolated nucleic acid molecule encoding an amino acid sequence encoded by a nucleic acid of the invention, wherein the nucleic acid is gene-optimized for expression in mammalian cells.

There is also provided according to the invention an isolated nucleic acid molecule encoding a polypeptide of the invention.

There is also provided according to the invention an isolated nucleic acid molecule encoding a polypeptide of the invention, wherein the nucleic acid is codon-optimized for expression in mammalian cells.

There is also provided according to the invention an isolated nucleic acid molecule encoding a polypeptide of the invention, wherein the nucleic acid is gene-optimized for expression in mammalian cells.

There is also provided according to the invention a vector comprising a nucleic acid of the invention.

Optionally the vector further comprises a promoter operably linked to the nucleic acid.

Optionally the promoter is for expression of a polypeptide encoded by the nucleic acid in mammalian cells.

Optionally the promoter is for expression of a polypeptide encoded by the nucleic acid in yeast, bacterial, or insect cells.

Optionally the vector is a vaccine vector. Optionally the vaccine vector is a viral vaccine vector, a bacterial vaccine vector, or a nucleic acid vector (for example an RNA vaccine vector, or a DNA vaccine vector).

A nucleic acid molecule of the invention may comprise a DNA or an RNA molecule. For embodiments in which the nucleic acid molecule comprises an RNA molecule, it will be appreciated that the molecule may comprise an RNA sequence that is at least 75%, 80%, 85%, 90%, 95%, 96%, 97%, 98%, or 99% identical with, or identical with, any of SEQ ID NOs: 1, 2, 4, 5, 7, 8, 10, 12, 14, 19, 21, 23, 25, 27, 29, or 31, in which each ‘T’ nucleotide is replaced by ‘U’, or the complement thereof.

For example, it will be appreciated that where an RNA vaccine vector comprising a nucleic acid of the invention is provided, the nucleic acid sequence of the nucleic acid of the invention will be an RNA sequence, so may comprise for example an RNA nucleic acid sequence that is at least 75%, 80%, 85%, 90%, 95%, 96%, 97%, 98%, or 99% identical with, or identical with, any of SEQ ID NOs: 1, 2, 4, 5, 7, 8, 10, 12, 14, 19, 21, 23, 25, 27, 29, or 31 in which each ‘T’ nucleotide is replaced by ‘U’, or the complement thereof.

There is also provided according to the invention an isolated cell comprising or transfected with a vector of the invention.

There is also provided according to the invention a virus pseudotype particle comprising a polypeptide of the invention.

According to the invention there is also provided a method of producing a virus pseudotype particle which includes transfecting a host cell with a vector comprising a nucleic acid of the invention.

There is also provided according to the invention a fusion protein comprising a polypeptide of the invention.

There is further provided according to the invention a pharmaceutical composition comprising a nucleic acid of the invention, and a pharmaceutically acceptable carrier, excipient, or diluent.

There is also provided according to the invention a pharmaceutical composition comprising a vector of the invention, and a pharmaceutically acceptable carrier, excipient, or diluent.

There is also provided according to the invention a pharmaceutical composition comprising a polypeptide of the invention, and a pharmaceutically acceptable carrier, excipient, or diluent.

Optionally a pharmaceutical composition of the invention further comprises an adjuvant for enhancing an immune response in a subject to the polypeptide, or to a polypeptide encoded by the nucleic acid, of the composition.

There is also provided according to the invention a method of inducing an immune response to a pathogen in a subject, which comprises administering to the subject a nucleic acid of the invention, a polypeptide of the invention, a vector of the invention, or a pharmaceutical composition of the invention.

Optionally the pathogen is a virus. Optionally the virus is a member of the Filoviridae, Arenaviridae, or Orthomyxoviridae family.

There is also provided according to the invention a method of inducing an immune response to a virus of the Filoviridae or Arenaviridae family in a subject, which comprises administering to the subject a nucleic acid of the invention, a polypeptide of the invention, a vector of the invention, or a pharmaceutical composition of the invention.

There is also provided according to the invention a method of immunizing a subject against a pathogen, which comprises administering to the subject a nucleic acid of the invention, a polypeptide of the invention, a vector of the invention, or a pharmaceutical composition of the invention.

Optionally the pathogen is a virus. Optionally the virus is a member of the Filoviridae, Arenaviridae, or Orthomyxoviridae family.

There is further provided according to the invention a method of immunizing a subject against a virus of the Filoviridae family, which comprises administering to the subject a nucleic acid of the invention, a polypeptide of the invention, a vector of the invention, or a pharmaceutical composition of the invention.

There is also provided according to the invention a method of inducing an immune response to a virus of the Filoviridae family in a subject, which comprises administering to the subject a nucleic acid of the invention, a polypeptide of the invention, a vector of the invention, or a pharmaceutical composition of the invention.

Optionally the nucleic acid, vector, or pharmaceutical composition of the invention comprises a nucleic acid comprising a sequence that is at least 75%, 80%, 85%, 90%, 95%, 96%, 97%, 98%, or 99% identical with, or identical with, any of SEQ ID NOs: 1, 2, 4, 5, 7, 8, 10, 12, or 14, or comprises a nucleic acid encoding an amino acid sequence encoded by a nucleic acid comprising a sequence that is at least 75%, 80%, 85%, 90%, 95%, 96%, 97%, 98%, or 99% identical with, or identical with, any of SEQ ID NOs: 1, 2, 4, 5, 7, 8, 10, 12, or 14.

Optionally the polypeptide, vector, or pharmaceutical composition of the invention comprises a polypeptide comprising an amino acid sequence that is at least 95%, 96%, 97%, 98%, or 99% identical with, or identical with, an amino acid sequence encoded by any of SEQ ID NOs: 1, 2, 4, 5, 7, 8, 10, 12, or 14, or comprises a polypeptide comprising an amino acid sequence that is at least 95%, 96%, 97%, 98%, or 99% identical with, or identical with, any of SEQ ID NOs: 3, 6, 9, 11, 13, or 15.

There is further provided according to the invention a method of immunizing a subject against a virus of the Arenaviridae family, which comprises administering to the subject a nucleic acid of the invention, a polypeptide of the invention, a vector of the invention, or a pharmaceutical composition of the invention.

There is also provided according to the invention a method of inducing an immune response to a virus of the Arenaviridae family in a subject, which comprises administering to the subject a nucleic acid of the invention, a polypeptide of the invention, a vector of the invention, or a pharmaceutical composition of the invention.

Optionally the nucleic acid, vector, or pharmaceutical composition of the invention comprises a nucleic acid comprising a sequence that is at least 75%, 80%, 85%, 90%, 95%, 96%, 97%, 98%, or 99% identical with, or identical with, any of SEQ ID NOs: 19, 21, 23, 25, 27, 29, or 31, or comprises a nucleic acid encoding an amino acid sequence encoded by a nucleic acid comprising a sequence that is at least 75%, 80%, 85%, 90%, 95%, 96%, 97%, 98%, or 99% identical with, or identical with, any of SEQ ID NOs: 19, 21, 23, 25, 27, 29, or 31.

Optionally the polypeptide, vector, or pharmaceutical composition of the invention comprises a polypeptide comprising an amino acid sequence that is at least 95%, 96%, 97%, 98%, or 99% identical with, or identical with, an amino acid sequence encoded by any of SEQ ID NOs: 19, 21, 23, 25, 27, 29, or 31, or comprises a polypeptide comprising an amino acid sequence that is at least 95%, 96%, 97%, 98%, or 99% identical with, or identical with, any of SEQ ID NOs: 18, 20, 22, 24, 26, 28, or 30.

Any suitable route of administration may be used. Methods of administration include, but are not limited to, intradermal, intramuscular, intraperitoneal, parenteral, intravenous, subcutaneous, vaginal, rectal, intranasal, inhalation or oral. Parenteral administration, such as subcutaneous, intravenous or intramuscular administration, is generally achieved by injection. Injectables can be prepared in conventional forms, either as liquid solutions or suspensions, solid forms suitable for solution or suspension in liquid prior to injection, or as emulsions. Injection solutions and suspensions can be prepared from sterile powders, granules, and tablets of the kind previously described. Administration can be systemic or local.

Compositions may be administered in any suitable manner, such as with pharmaceutically acceptable carriers. Pharmaceutically acceptable carriers are determined in part by the particular composition being administered, as well as by the particular method used to administer the composition. Preparations for parenteral administration include sterile aqueous or nonaqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Administration can be accomplished by single or multiple doses. The dose administered to a subject in the context of the present disclosure should be sufficient to induce a beneficial therapeutic response in a subject over time, or to inhibit or prevent infection. The dose required will vary from subject to subject depending on the species, age, weight and general condition of the subject, the severity of the infection being treated, the particular composition being used and its mode of administration. An appropriate dose can be determined by one of ordinary skill in the art using only routine experimentation.

Pharmaceutically acceptable carriers include, but are not limited to, saline, buffered saline, dextrose, water, glycerol, ethanol, and combinations thereof. The carrier and composition can be sterile, and the formulation suits the mode of administration. The composition can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulations can include standard carriers such as pharmaceutical grades of mannitol, lactose, starch, magnesium stearate, sodium saccharine, cellulose, and magnesium carbonate. Any of the common pharmaceutical carriers, such as sterile saline solution or sesame oil, can be used. The medium can also contain conventional pharmaceutical adjunct materials such as, for example, pharmaceutically acceptable salts to adjust the osmotic pressure, buffers, preservatives and the like. Other media that can be used with the compositions and methods provided herein are normal saline and sesame oil.

In some embodiments, the compositions comprise a pharmaceutically acceptable carrier and/or an adjuvant. For example, the adjuvant can be alum, Freund's complete adjuvant, a biological adjuvant or immunostimulatory oligonucleotides (such as CpG oligonucleotides).

The pharmaceutically acceptable carriers (vehicles) useful in this disclosure are conventional. Remington's Pharmaceutical Sciences, by E. W. Martin, Mack Publishing Co., Easton, PA, 15th Edition (1975), describes compositions and formulations suitable for pharmaceutical delivery of one or more therapeutic compositions, such as one or more influenza vaccines, and additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Optionally a composition of the invention is administered intramuscularly.

Optionally the composition is administered intramuscularly, intradermaly, subcutaneously by needle or by gene gun, or electroporation.

There is also provided according to the invention a nucleic acid expression vector, which comprises a multiple cloning site, comprising KpnI and NotI endonuclease sites.

Optionally the multiple cloning site comprises a nucleic acid sequence of SEQ ID NO: 16.

Optionally the nucleic acid expression vector is a nucleic acid expression vector, and a viral pseudotype vector.

Optionally the nucleic acid expression vector is a vaccine vector.

Optionally the nucleic acid expression vector comprises, from a 5′ to 3′ direction: a promoter; a splice donor site (SD); a splice acceptor site (SA); and a terminator signal, wherein the multiple cloning site is located between the splice acceptor site and the terminator signal.

Optionally the promoter comprises a CMV immediate early 1 enhancer/promoter (CMV-IE-E/P) and/or the terminator signal comprises a terminator signal of a bovine growth hormone gene (Tbgh) that lacks a KpnI restriction endonuclease site.

Optionally the nucleic acid expression vector further comprises an origin of replication, and nucleic acid encoding resistance to an antibiotic. Optionally the origin of replication comprises a pUC-plasmid origin of replication and/or the nucleic acid encodes resistance to kanamycin.

Optionally the nucleic acid expression vector comprises a nucleic acid sequence of SEQ ID NO:17 (pEVAC).

A polypeptide of the invention may include one or more conservative amino acid substitutions. Conservative amino acid substitutions are those substitutions that, when made, least interfere with the properties of the original protein, that is, the structure and especially the function of the protein is conserved and not significantly changed by such substitutions. Examples of conservative substitutions are shown below:

| Oriainal Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain.

The substitutions which in general are expected to produce the greatest changes in protein properties will be non-conservative, for instance changes in which (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histidyl, is substituted for (or by) an electronegative residue, for example, glutamyl or aspartyl; or (d) a residue having a bulky side chain, for example, phenylalanine, is substituted for (or by) one not having a side chain, for example, glycine.

In particular embodiments of the invention sequence alignments and ancestral sequence reconstruction (ASR) are used to identify highly conserved immune targets which the pathogens cannot change and which will invariably be present in future outbreaks of that viral family, even in the most highly variable RNA viruses. Synthetic gene technology is used to produce computer generated virus genes so that they are highly expressed and can be easily cloned into an expression vector, such as the pEVAC vector (one that has proven to be a highly versatile expression vector for generating viral pseudotypes as well as direct DNA vaccination of animals and or humans).

Large panels of genes can be generated using the pEVAC vector so that viral pseudotypes are rapidly generated. This allows a library of viral pseudotypes, each with its own unique viral protein to be probed with a large panel of monoclonal antibodies. This process ensures that the inserts generate conformation correct viral surface proteins to present the most accessible target to a viral Achilles heel. The down-selection of candidates by pseudotype formation and mAb binding provides a shortlist of top candidates to test by vaccination. This may be done in Guinea pigs where the streamlined process of pEVAC-vaccine inserts are delivered. If required this enables the shuttle of vaccine inserts from the DNA pEVAC vector into a variety of viral vectors based on advanced designed convenient cloning sites. Since Chimpanzee Adenovectors (ChAd) were widely used in evaluating the majority of Ebola virus vaccine candidates in phase I for the West African outbreak, we chose to compare to use the same vector for head to head comparison in humans. For screening in Guinea pigs we used DNA priming with pEVAC-vaccine insert followed by ChAd-vaccine insert.

In particular embodiments of the invention:
1) High throughput “deep” sequencing technology provides viral variation data from current and past outbreaks. By analysing this data, structural, highly conserved regions can be identified which can be used as scaffolds for designing optimal vaccine inserts, and which preserve known B and T cell epitopes.
2) Human monoclonal antibody (mAb) technology allows the generation of anti-viral mAbs to vaccine targets, such as the virus envelope protein, which identify the epitope rich regions to which broadly neutralising monoclonal antibodies (BNmAbs) target.
3) Optimal gene design and synthesis incorporates the digitally modelled conserved scaffolds of genes identified in (1), to include the broadest NmAb epitopes on these scaffolds (BN epitopes are likely not to be optimally presented on naturally conserved GPs).
4) Downstream knowledge of convenient cloning sites matching the requirements of vaccine and pseudotype vectors are taken into account during the design and synthesis of RNA and codon optimised synthetic genes as vaccine inserts to enable rapid and highly efficient cloning and shuttling into different screening (i.e. lentiviral pseudotypes; PVs) and vaccine (i.e. MVA, ChAd, VSV, DNA etc) vectors.
5) Viral pseudotypes (lentiviral) generated from digitally designed inserts are screened in vitro for functionality via transduction and infection studies. Further to this, neutralisation assays using a panel of BNmAbs and patient sera is undertaken to ensure that known epitopes are preserved.
6) Down-selection of several synthetic vaccine inserts to the best-in-class vaccine inserts are confirmed for immunogenicity in guinea pigs, using rapid DNA priming (and if required) with adenovirus boosting, a method that gives high and reproducible titres. In vivo screening confirms which are the most immunogenic and give the greatest neutralisation breadth.

The central role of the viral glycoprotein in cell attachment, fusion and uncoating make it a key antigenic target for viral vaccines and monoclonal antibody therapies that have been pioneered during the West African Ebola outbreak. Analysis of the GP sequences between species of EBOV showed a high degree of diversity at the nucleotide and amino acid level (only ~60-65% nt identity). For current conventional Filovirus vaccine approaches, GP targeting vaccines need to be multivalent, encoding GPs specific for each species, which are more conserved (~97-98% identity in the GP nucleotide sequence). Although it has been suggested that vaccines using older strains of EBOV (rVSV.ZEBOV=Kikwit) may provide cross-protection (Henao-Restrepo A M, Lancet 2015), there is concern that this may have limited efficacy against future outbreaks of other diverse highly pathogenic Filoviruses.

We can achieve dramatic improvements in vaccine efficacy against new viral variants based on sequence data (optionally including, for example, outbreak sequence data) to generate synthetic optimised vaccine inserts to give the broadest possible vaccine protection against future outbreaks of variable RNA viruses. In particular embodiments, our new vaccine technology merges:
(1) Sequences of outbreak pathogens
(2) Broadly anti-viral neutralising monoclonal antibodies (BNmAb) derived from outbreak survivors
(3) Computational modelling methodologies
(4) Synthetic gene technology and antigen display technology
(5) High-throughput viral binding and neutralisation screens
(6) In vivo immune selection and vaccine efficacy readouts

The end products are novel immunogens used to trigger the broadest spectrum of protective immune responses. We have provided proof of concept that the next generation single vaccine inserts do induce broad neutralisation profiles against the Ebolavirus genus (Zaire, Sudan, Bundibugyo), additionally targeting the more distant filovirus, Marburg virus.

Embodiments of the invention are described, by way of illustration only, in the Examples below, with reference to the accompanying drawings in which:

FIG. 1 shows an illustration of a phylogenetic tree and its relation to ancestral sequence reconstruction;

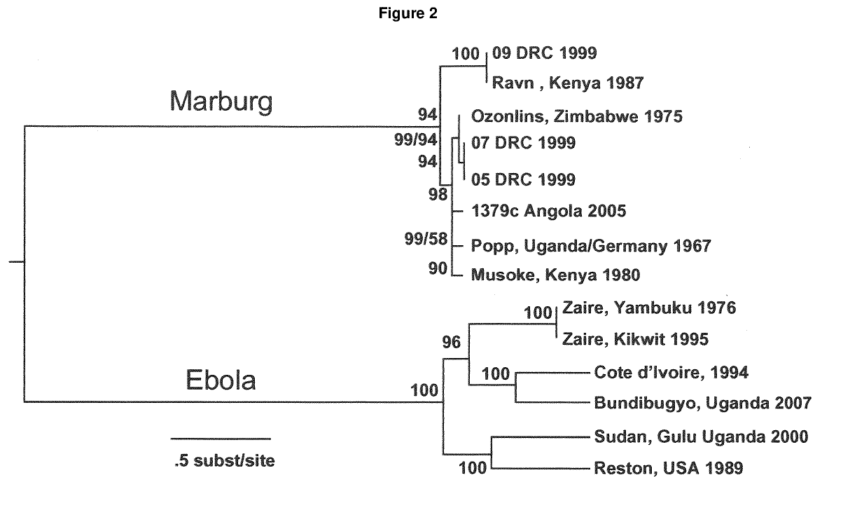

FIG. 2 shows a phylogenetic tree comparing ebolaviruses and Marburg viruses. Numbers indicate percent confidence of branches;

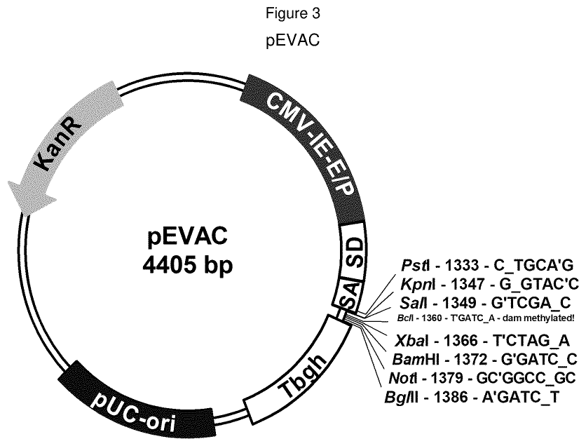

FIG. 3 shows a plasmid map for pEVAC;

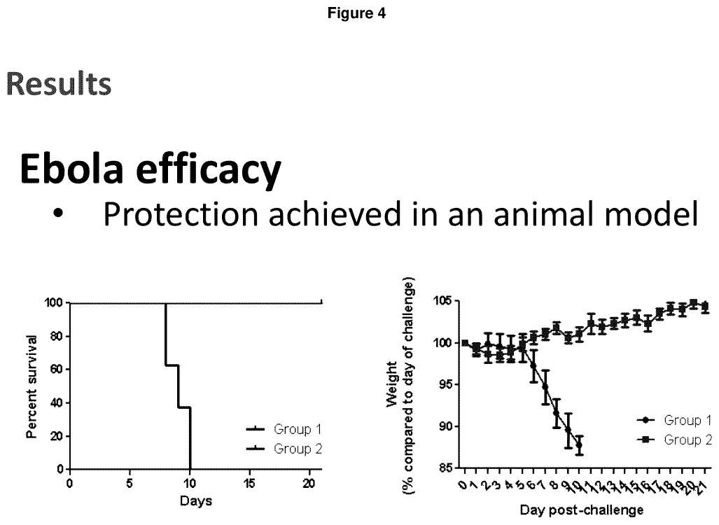

FIG. 4 shows challenge study results for an Ebola challenge model. Ebola challenge model was lethal for non-vaccinated guinea pigs (Group 1, lower line) whereas all vaccinated guinea pigs (Group 2, upper line) were protected (left) and continued to gain weight (right);

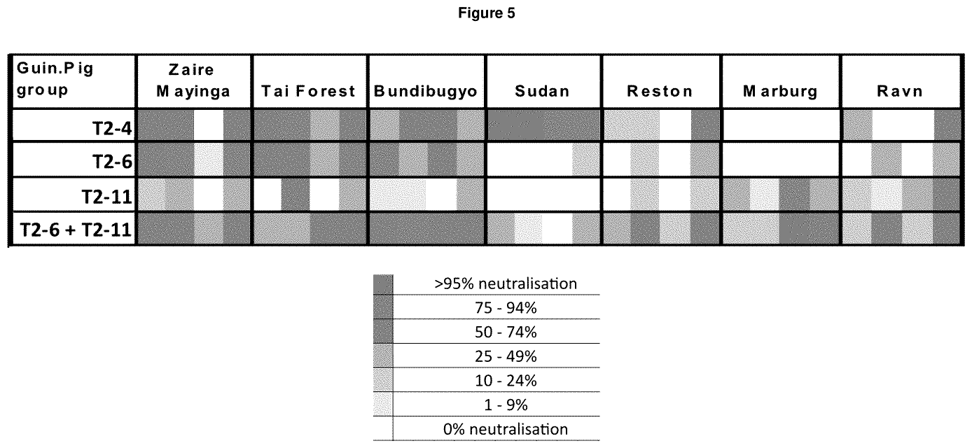

FIG. 5 shows the results of a pseudotype virus neutralisation assay illustrating the strength of neutralising antibody responses to target antigens expressed on the surface of a pseudotyped virus, representative of all Ebola virus species and Marburg viruses. Strength of neutralisation is indicated by the heat-map where red (darkest shading) is very strong neutralisation, decreasing through orange to yellow (progressively lighter shading) and no neutralising/equal to negative control values are white. T2-4 and T2-6 are nucleic acid vaccines encoding lead candidate optimized antigenic Ebola polypeptide, combined with T2-11 a Marburg candidate, at pre-clinical stage testing with serum samples taken from immunised guinea pigs;

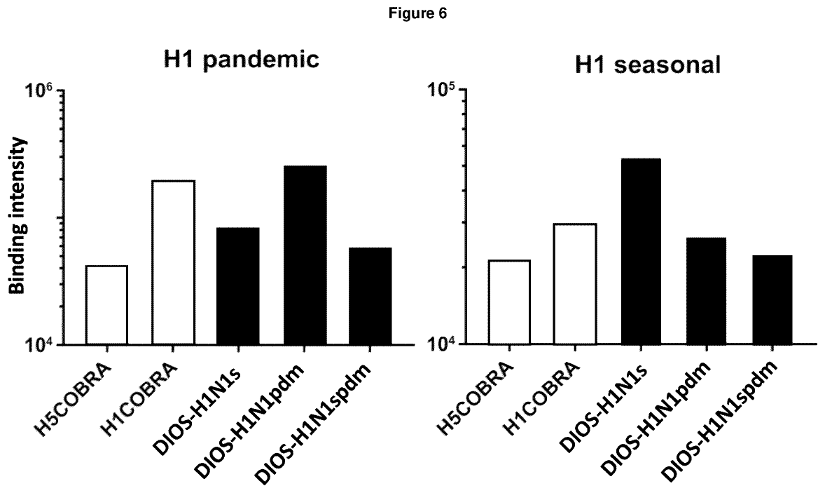

FIG. 6 shows the results of study to determine the effectiveness of nucleic acid vaccines encoding different lead candidate optimized antigenic pathogenic polypeptides, identified using an embodiment of a method of the invention. Antibody binding was measured by incubation of two groups of cells bearing two different group 1 influenza A glycoproteins on their surface (H1 pandemic and seasonal) with pooled mouse serum. Any bound antibodies were then detected by a secondary antibody, and results recorded using a flow cytometer. Binding was significantly increased before and after vaccination with all constructs, but not after vaccination with PBS (control). Overall, a vaccine candidate out-performed those from COBRA in both cases (*);

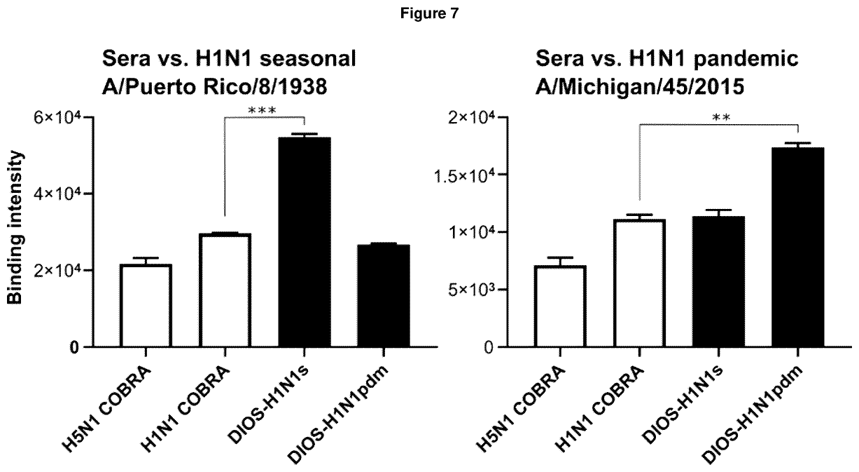

FIG. 7 shows the results of a study to determine binding of cells expressing two different group 1 influenza A glycoproteins on their cell surface (seasonal H1N1, and pandemic origin H1N1) by mouse sera from animals immunized with either the COBRA or DIOS HA gene antigens; and

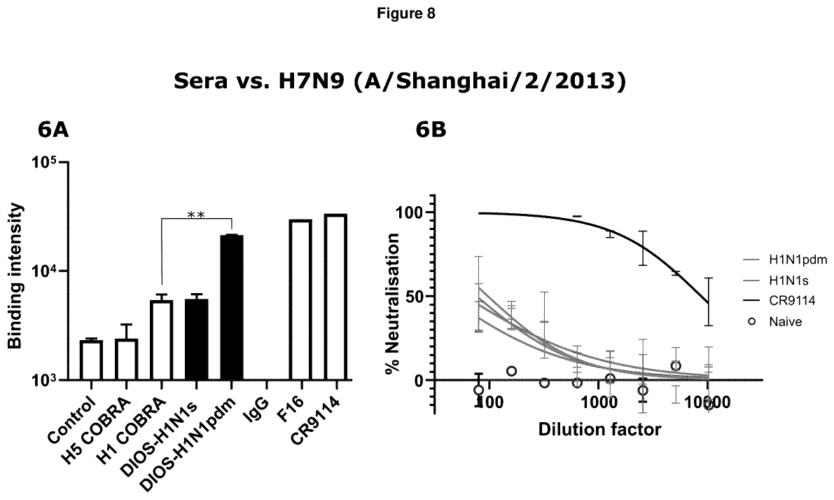

FIG. 8 shows the results of cross-HA-group binding (left panel), and pseudotype neutralization (right) of H7N9 (A/Shanghai2/2013), by sera from DIOS or COBRA DNA immunized mice. In the right panel, the uppermost curve is for CR9114, the two curves falling from the lowest two starting points at the left of the graph are for H1N1s, and the remaining two curves are for H1N1pdm.

Examples of unoptimized Ebola and Marburg viral ancestral nucleic acid sequences (i.e. sequences which have not been codon-optimized or gene-optimized) are given below, as well as gene-optimized nucleic acid sequences encoding candidate antigenic pathogen polypeptides.

### Methodology

For a given virus species, candidate primary sequences are downloaded, for example, from GenBank (and from any other available sources, such as outbreak data), and are filtered to remove identical sequences, sequences that do not span the protein of interest, and sequences that have a high number of ambiguous nucleotides. A multiple sequence alignment of the filtered sequences is generated (typically using MAFFT), and checked manually to ensure that sequences are in the correct open reading frame. A maximum likelihood phylogeny is generated using IQTREE, with automated model selection, and rooted using one of several methods; an outgroup sequence, midpoint rooting, centre-of-the-tree, or a tree that maximises the association between root-to-tip distance and sampling time. Ancestral sequences are generated using HyPhy assuming a MG94 by F3x4 model of codon substitution, and are checked to ensure that known epitopes have been preserved. A phylogenetic tree with both primary and ancestral sequences is generated using IQTREE to check the placement of the ancestral strains. Ancestral sequences are then modified in a number of ways: deletion of regions (e.g. removal of the mucin-like domain); region swapping (to recover potential lost epitopes); mutation of specific sites (e.g. in the fusion domain of the filoviruses), including editing of N-linked glycosylation sites and introduction of mutations to enhance stability.

### Example 1

Ebola Sudan ancestor (T2-4)
Unoptimised (SEQ ID NO: 1)
ATGGGGGGTCTTAGCCTACTCCAATTGCCCAGGGACAAATTTCGGAAAAG
CTCTTTCTTTGTTTGGGTCATCATCTTATTCCAAAAGGCCTTTTCCATGC
CTTTGGGTGTTGTGACTAACAGCACTTTAGAAGTAACAGAGATTGACCAG
CTAGTCTGCAAGGATCATCTTGCATCCACTGACCAGCTGAAATCAGTTGG
TCTCAACCTCGAGGGGAGCGGAGTATCTACTGATATCCCATCTGCAACAA
AGCGTTGGGGCTTCAGATCTGGTGTTCCTCCCAAGGTGGTCAGCTATGAA
GCGGGAGAATGGGCTGAAAATTGCTACAATCTTGAAATAAAGAAGCCGGA
CGGGAGCGAATGCTTACCCCCACCGCCAGATGGTGTCAGAGGCTTTCCAA
GGTGCCGCTATGTTCACAAAGCCCAAGGAACCGGGCCCTGCCCAGGTGAC
TACGCCTTTCACAAGGATGGAGCTTTCTTCCTCTATGACAGGCTGGCTTC
AACTGTAATTTACAGAGGAGTCAATTTTGCTGAGGGGGTAATTGCATTCT
TGATATTGGCTAAACCAAAAGAAACGTTCCTTCAGTCACCCCCCATTCGA
GAGGCAGTAAACTACACTGAAAATACATCAAGTTATTATGCCACATCCTA
CTTGGAGTATGAAATCGAAAATTTTGGTGCTCAACACTCCACGACCCTTT
TCAAAATTGACAATAATACTTTTGTTCGTCTGGACAGGCCCCACACGCCT
CAGTTCCTTTTCCAGCTGAATGATACCATTCACCTTCACCAACAGTTGAG
CAACACAACTGGGAGACTAATTTGGACACTAGATGCTAATATCAATGCTG
ATATTGGTGAATGGGCTTTTTGGGAAAATAAAAAAAATCTCTCCGAACAA
CTACGTGGAGAAGAGCTGTCTTTCGAAGCTTTATCGCTCACAACAGCGGT
TAAAACTGTCTTGCCACAGGAGTCCACAAGCAACGGTCTAATAACTTCAA
CAGTAACAGGGATTCTTGGGAGTCTTGGGCTTCGAAAACGCAGCAGAAGA
CAAGTTAACACCAAAGCCACGGGTAAATGCAATCCCAACTTACACTACTG
GACTGCACAAGAACAACATAATGCTGCTGGGATTGCCTGGATCCCGTACT
TTGGACCGGGTGCGGAAGGCATATACACTGAAGGCCTGATGCATAACCAA
AATGCCTTAGTCTGTGGACTTAGGCAACTTGCAAATGAAACAACTCAAGC
TCTGCAGCTTTTCTTAAGAGCCACAACGGAGCTGCGGACATATACCATAC
TCAATAGGAAGGCCATAGATTTCCTTCTGCGACGATGGGGCGGGACATGC
AGGATCCTGGGACCAGATTGTTGCATTGAGCCACATGATTGGACAAAAAA
CATCACTGATAAAATCAACCAAATCATCCATGATTTCATCGACAACCCCT
TACCTAATCAGGATAATGATGATAATTGGTGGACGGGCTGGAGACAGTGG
ATCCCTGCAGGAATAGGCATTACTGGAATTATTATTGCAATTATTGCTCT
TCTTTGCGTTTGCAAGCTGCTTTGCTAG
Gene-optimised (SEQ ID NO: 2)
ATGGGAGGACTGTCTCTGCTGCAACTGCCCCGGGACAAGTTCCGGAAGTC
CAGCTTCTTCGTGTGGGTCATCATCCTGTTCCAGAAAGCCTTCAGCATGC
CCCTGGGCGTCGTGACCAATAGCACACTGGAAGTGACCGAGATCGACCAG
CTCGTGTGCAAGGATCACCTGGCCAGCACCGATCAGCTGAAGTCTGTGGG
ACTGAATCTGGAAGGCAGCGGCGTGTCCACAGATATCCCTAGCGCCACCA
AGAGATGGGGCTTTAGAAGCGGAGTGCCTCCTAAGGTGGTGTCTTATGAA
GCCGGCGAGTGGGCCGAGAACTGCTACAACCTGGAAATCAAGAAGCCCGA
CGGCAGCGAGTGTCTGCCTCCTCCACCTGATGGCGTCAGAGGCTTCCCTA
GATGCAGATACGTGCACAAGGCCCAAGGCACAGGACCCTGTCCTGGCGAT
TACGCCTTTCACAAGGACGGCGCCTTTTTCCTGTACGATCGGCTGGCCTC
CACCGTGATCTACAGAGGCGTTAACTTTGCCGAGGGCGTGATCGCCTTCC
TGATCCTGGCCAAGCCTAAAGAGACATTCCTGCAAAGCCCTCCAATCCGC
GAGGCCGTGAACTACACAGAGAACACCAGCAGCTACTACGCCACCAGCTA
CCTGGAATACGAGATCGAGAATTTCGGCGCCCAGCACAGCACCACACTGT
TCAAGATCGACAACAACACCTTCGTGCGGCTGGACAGACCCCACACACCT
CAGTTTCTGTTCCAGCTGAACGACACCATCCATCTGCATCAGCAGCTGAG
CAACACCACCGGCAGACTGATTTGGACCCTGGACGCCAACATCAACGCCG
ACATTGGAGAGTGGGCCTTTTGGGAGAACAAGAAGAACCTGAGCGAACAG
CTGAGAGGCGAGGAACTGAGCTTTGAGGCCCTGTCTCTGACCACCGCCGT
GAAAACAGTGCTGCCTCAAGAGTCCACCAGCAACGGCCTGATCACAAGCA
CAGTGACAGGCATCCTGGGCAGCCTGGGCCTGAGAAAAAGGTCCAGACGG
CAAGTGAATACCAAGGCCACCGGCAAGTGCAACCCCAACCTGCACTATTG
GACAGCCCAAGAGCAGCACAATGCCGCCGGAATCGCCTGGATTCCTTATT
TTGGACCTGGCGCCGAGGGCATCTATACCGAGGGACTGATGCACAACCAG
AACGCCCTCGTGTGTGGACTGAGACAGCTGGCCAATGAGACAACACAGGC
CCTCCAGCTGTTTCTGAGAGCCACCACCGAGCTGAGAACCTACACCATCC
TGAACCGGAAGGCCATCGACTTTCTGCTGAGAAGATGGGGCGGCACCTGT
AGAATCCTGGGACCTGATTGCTGCATCGAGCCCCACGACTGGACCAAGAA
CATCACCGACAAGATCAACCAGATCATCCACGACTTCATCGACAACCCTC
TGCCTAACCAGGACAACGACGACAATTGGTGGACAGGCTGGCGGCAGTGG
ATTCCTGCCGGAATTGGCATCACCGGCATCATCATTGCCATTATCGCCCT
GCTGTGTGTGTGCAAGCTGCTGTGTTGA
Amino acid sequence encoded by unoptimised and
gene-optimised sequences (SEQ ID NO: 3):
MGGLSLLQLPRDKERKSSFEVWVIILFQKAFSMPLGVVTNSTLEVTEIDQ
LVCKDHLASTDQLKSVGLNLEGSGVSTDIPSATKRWGFRSGVPPKVVSYE
AGEWAENCYNLEIKKPDGSECLPPPPDGVRGFPRCRYVHKAQGTGPCPGD
YAFHKDGAFFLYDRLASTVIYRGVNFAEGVIAFLILAKPKETFLQSPPIR
EAVNYTENTSSYYATSYLEYEIENFGAQHSTTLFKIDNNTEVRLDRPHTP
QFLFQLNDTIHLHQQLSNTTGRLIWTLDANINADIGEWAFWENKKNLSEQ
LRGEELSFEALSLTTAVKTVLPQESTSNGLITSTVTGILGSLGLRKRSRR
QVNTKATGKCNPNLHYWTAQEQHNAAGIAWIPYFGPGAEGIYTEGLMHNQ
NALVCGLRQLANETTQALQLFLRATTELRTYTILNRKAIDFLLRRWGGTC
RILGPDCCIEPHDWTKNITDKINQIIHDFIDNPLPNQDNDDNWWTGWRQW
IPAGIGITGIIIAIIALLCVCKLLC

### Example 2

Ebolavirus global ancestor (T2-6)
Unoptimised (SEQ ID NO: 4)
ATGGGGGGTGGATCCAGACTTCTCCAATTGCCCCGGGAACGCTTTCGGAA
AACCTCATTCTTTGTTTGGGTAATCATCCTATTCCAAAAAGCCTTTTCCA
TGCCATTGGGTGTTGTAACCAACAGCACTCTAAAAGTAACAGAAATTGAC
CAATTGGTTTGCCGGGACAAACTTTCATCCACAAGTCAGCTGAAATCAGT
TGGGCTGAATCTGGAAGGGAATGGAGTTGCAACTGATGTCCCATCAGCAA
CAAAACGATGGGGCTTCCGATCTGGTGTTCCTCCCAAGGTGGTCAGCTAT
GAAGCTGGAGAATGGGCTGAAAATTGCTACAATCTGGAAATCAAGAAGCC
AGACGGGAGTGAATGCCTACCTCCACCGCCAGACGGTGTAAGAGGCTTCC
CCAGGTGCCGCTATGTCCACAAAGTTCAAGGAACAGGGCCGTGTCCTGGT
GACTTCGCCTTCCACAAAGATGGAGCTTTCTTCCTGTATGATAGACTGGC
TTCAACTGTCATTTACCGAGGGACAACTTTTGCTGAAGGTGTCGTTGCAT
TTTTGATCCTGCCCAAACCTAAAAAGGACTTTTTCCAATCACCCCCAATA
CGTGAGCCGGTAAACACCACAGAAGATCCATCAAGTTACTACACCACATC
AACACTTAGCTATGAGATTGACAATTTTGGGGCCAATAAAACTAAAACTC
TTTTCAAAGTTGACAATCACACTTATGTGCAACTAGACCGACCACACACA
CCACAGTTCCTTGTCCAGCTCAATGAAACCATTCATACAAATAACCGTCT
AAGCAACACCACAGGGAGACTAATTTGGACATTAGATCCTAAAATTGATA
CCGACATTGGTGAGTGGGCCTTCTGGGAAAATAAAAAAAACTTCTCCAAA
CAACTTCGTGGAGAAGAGTTGTCTTTCAAAGCTCTATCAACAAAAACTGG
AGCTAACGCAGTAGACACTGACGAATCAAGCAAACCTGGCCTAATTACCA
ACACAGTAAGAGGGGTTGCTGATTTACTGAGCCCTTGGAGAAGAAAAAGA
AGACAAGTCAACCCAAACACAACAAATAAATGCAACCCAAACCTACACTA
TTGGACAGCCCAAGATGAAGGTGCTGCCGTTGGATTAGCCTGGATCCCAT
ACTTCGGACCAGCAGCAGAAGGCATTTACACTGAAGGAATAATGCATAAT
CAAAATGGGTTAATCTGTGGGCTGAGGCAGCTGGCCAATGAAACGACTCA
AGCTCTTCAATTATTCTTGAGGGCCACAACGGAGCTGCGGACTTACTCTA
TACTCAATAGAAAAGCCATTGATTTCCTTCTCCAACGATGGGGAGGAACA
TGCCGCATCTTAGGACCAGATTGTTGCATTGAGCCACATGATTGGACAAA
AAACATTACTGATAAAATTAACCAAATCATACATGATTTTATTGACAACC
CTCTACCAGATCAGGACGATGATGACAATTGGTGGACAGGCTGGAGACAA
TGGATCCCTGCTGGAATTGGAATTACTGGAGTTATAATTGCAATTATAGC
TCTACTTTGTATTTGCAAGTTTCTGTGTTAG
Gene-optimised (SEQ ID NO: 5)
ATGGGCGGAGGATCTAGACTGCTGCAACTGCCCAGAGAGCGGTTCAGAAA
GACCAGCTTCTTCGTGTGGGTCATCATCCTGTTCCAGAAAGCCTTCAGCA
TGCCCCTGGGCGTCGTGACCAATAGCACCCTGAAAGTGACCGAGATCGAC
CAGCTCGTGTGCAGAGATAAGCTGAGCAGCACCAGCCAGCTGAAGTCCGT
GGGACTGAATCTGGAAGGCAATGGCGTGGCCACAGATGTGCCTAGCGCCA
CCAAAAGATGGGGCTTTAGAAGCGGCGTGCCACCTAAGGTGGTGTCTTAT
GAAGCCGGCGAGTGGGCCGAGAACTGCTACAACCTGGAAATCAAGAAGCC
CGACGGCAGCGAGTGTCTGCCTCCTCCACCTGATGGCGTCAGAGGCTTCC
CTAGATGCAGATACGTGCACAAGGTGCAAGGCACAGGCCCCTGTCCTGGC
GATTTCGCCTTTCACAAGGACGGCGCCTTTTTCCTGTACGATCGGCTGGC
CTCCACCGTGATCTACAGAGGCACAACATTTGCCGAAGGCGTGGTGGCCT
TCCTGATCCTGCCTAAGCCTAAGAAGGACTTCTTTCAGAGCCCTCCTATC
CGCGAGCCTGTGAACACAACAGAGGACCCCAGCAGCTACTACACCACCAG
CACACTGAGCTACGAGATCGATAACTTCGGCGCCAACAAGACCAAGACAC
TGTTCAAGGTGGACAACCACACCTACGTGCAGCTGGACAGACCCCACACA
CCTCAGTTTCTGGTGCAGCTGAACGAGACAATCCACACCAACAACAGACT
GAGCAACACCACCGGCAGGCTGATCTGGACCCTGGATCCTAAGATCGACA
CCGACATCGGAGAGTGGGCCTTTTGGGAGAACAAGAAGAACTTCAGCAAG
CAGCTGAGAGGCGAGGAACTGAGCTTTAAGGCCCTGAGCACCAAGACAGG
CGCCAACGCTGTGGATACCGATGAGTCTAGCAAGCCCGGCCTGATCACCA
ACACAGTTAGAGGCGTTGCCGACCTGCTGAGCCCTTGGAGAAGAAAGCGG
AGACAAGTGAACCCCAATACCACCAACAAGTGCAACCCTAACCTGCACTA
CTGGACAGCCCAGGATGAAGGCGCTGCTGTTGGACTGGCCTGGATTCCTT
ATTTTGGACCTGCCGCCGAGGGCATCTACACAGAGGGAATCATGCACAAC
CAGAATGGCCTGATCTGCGGCCTGAGACAGCTGGCCAATGAGACAACACA
GGCCCTCCAGCTGTTTCTGAGAGCCACCACCGAGCTGAGAACCTACAGCA
TCCTGAACCGGAAGGCCATCGACTTTCTGCTGCAAAGATGGGGAGGCACC
TGTAGAATCCTGGGACCTGATTGCTGCATCGAGCCCCACGACTGGACCAA
GAACATCACCGACAAGATCAACCAGATCATCCACGACTTCATCGACAACC
CTCTGCCTGACCAGGACGACGACGATAATTGGTGGACAGGATGGCGGCAG
TGGATTCCTGCCGGAATCGGAATCACAGGCGTGATCATTGCCATTATCGC
CCTGCTGTGCATCTGCAAGTTTCTGTGCTGA
Amino acid sequence encoded by unoptimised and
gene-optimised sequences (SEQ ID NO: 6):
MGGGSRLLQLPRERFRKTSFFVWVIILFQKAFSMPLGVVTNSTLKVTEID
QLVCRDKLSSTSQLKSVGLNLEGNGVATDVPSATKRWGFRSGVPPKVVSY
EAGEWAENCYNLEIKKPDGSECLPPPPDGVRGFPRCRYVHKVQGTGPCPG
DFAFHKDGAFFLYDRLASTVIYRGTTFAEGVVAFLILPKPKKDFFQSPPI
REPVNTTEDPSSYYTTSTLSYEIDNFGANKTKTLFKVDNHTYVQLDRPHT
PQFLVQLNETIHTNNRLSNTTGRLIWTLDPKIDTDIGEWAFWENKKNFSK
QLRGEELSFKALSTKTGANAVDTDESSKPGLITNTVRGVADLLSPWRRKR
RQVNPNTTNKCNPNLHYWTAQDEGAAVGLAWIPYFGPAAEGIYTEGIMHN
QNGLICGLRQLANETTQALQLFLRATTELRTYSILNRKAIDFLLQRWGGT
CRILGPDCCIEPHDWTKNITDKINQIIHDFIDNPLPDQDDDDNWWTGWRQ
WIPAGIGITGVIIAIIALLCICKFLC

### Example 3

Marburgvirus ancestor (T2-11)
Unoptimised (SEQ ID NO: 7)
ATGAAGACCATATATTTTCTGATTAGTCTCATTTTAATCCAAAGTATAAA
AACTCTCCCTGTTTTAGAAATTGCTAGTAACAGCCAACCTCAAGATGTAG
ATTCAGTGTGCTCCGGAACCCTCCAAAAGACAGAAGATGTTCATCTGATG
GGATTTACACTGAGTGGGCAAAAAGTTGCTGATTCCCCTTTGGAAGCATC
TAAACGATGGGCTTTCAGGACAGGTGTTCCTCCCAAGAACGTTGAGTATA
CGGAAGGAGAAGAAGCCAAAACATGTTACAATATAAGTGTAACAGACCCT
TCTGGAAAATCCTTGCTGCTGGATCCTCCCAGTAATATCCGCGATTACCC
TAAATGTAAAACTGTTCATCATATTCAAGGTCAAAACCCTCATGCACAGG
GGATTGCCCTCCATTTGTGGGGGGCATTTTTCCTGTATGATCGCATTGCC
TCCACAACAATGTACCGAGGCAAAGTCTTCACTGAAGGGAACATAGCAGC
TATGATTGTCAATAAGACAGTGCACAAAATGATTTTCTCGAGGCAAGGAC
AAGGGTACCGTCACATGAATCTGACTTCTACTAATAAATATTGGACAAGT
AGCAACGGAACGCAAACGAATGACACTGGATGCTTCGGTGCTCTTCAAGA
ATACAATTCTACGAAGAACCAAACATGTGCTCCGTCCAAAATACCTCCAC
CACTGCCCACAGCCCGTCCGGAGATCAAACCCACAAGCACCCCAACTGAT
GCCACCAAACTCAACACCACAGACCCAAACAGTGATGATGAGGACCTCAC
AACATCCGGCTCAGGGTCCGGAGAACAGGAACCCTACACAACTTCTGATG
CGGTCACTAAGCAAGGGCTTTCATCAACAATGCCACCCACTCCCTCACCA
CAACCAAGCACGCCACAGCAAGGAGGAAACAACACAAACCATTCCCAAGG
TGCTGTGACTGAACCCGACAAAACCAACACAACTGCACAACCGTCCATGC
CCCCCCACAACACTACTACAATCTCTACTAACAACACCTCCAAGCACAAC
TTCAGCACTCTCTCTGCACCACTACAAAACACCACCAATTACAACACACA
GAGCACGGCCACTGAAAATGAGCAAACCAGTGCCCCCTCGAAAACAACCC
TGCCTCCAACAGGAAATCCTACCACAGCAAAGAGCACCAACAGCACAAAA
GGCCCCACCACAACGGCACCAAATACGACAAATGGGCATTTCACCAGTCC
CTCCCCCACCCCCAACTCGACTACACAACATCTTGTATATTTCAGAAGGA
AACGAAGTATCCTCTGGAGGGAAGGCGACATGTTCCCTTTTTTAGATGGG
TTAATAAATACTGAAATTGATTTTGATCCAATCCCAAACACAGAAACAAT
CTTTGATGAATCCCCCAGCTTTAATACTTCAACTAATGAGGAACAACACA
CTCCCCCGAATATCAGTTTAACTTTCTCTTATTTTCCTGATAAAAATGGA
GATACTGCCTACTCTGGGGAAAACGAGAATGATTGTGATGCAGAGTTGAG
GATTTGGAGTGTGCAGGAGGACGATTTGGCGGCAGGGCTTAGCTGGATAC
CATTTTTTGGCCCTGGAATCGAAGGACTCTATACTGCCGGTTTAATCAAA
AATCAGAACAATTTAGTTTGTAGGTTGAGGCGCTTAGCTAATCAAACTGC
TAAATCCTTGGAGCTCTTGTTAAGGGTCACAACCGAGGAAAGGACATTTT
CCTTAATCAATAGGCATGCAATTGACTTTTTGCTTACGAGGTGGGGCGGA
ACATGCAAGGTGCTAGGACCTGATTGTTGCATAGGAATAGAAGATCTATC
TAAAAATATCTCAGAACAAATTGACAAAATCAGAAAGGATGAACAAAAGG
AGGAAACTGGCTGGGGTCTAGGTGGCAAATGGTGGACATCTGACTGGGGT
GTTCTCACCAATTTGGGCATCCTGCTACTATTATCTATAGCTGTTCTGAT
TGCTCTGTCCTGTATCTGTCGTATCTTCACTAAATATATCGGATAG
Gene-optimised (SEQ ID NO: 8)
ATGAAGACCATCTACTTTCTGATCAGCCTGATCCTGATCCAGAGCATCAA
GACCCTGCCTGTGCTGGAAATCGCCAGCAACAGTCAGCCCCAGGATGTGG
ATAGCGTGTGTAGCGGCACCCTCCAGAAAACCGAGGATGTGCACCTGATG
GGCTTTACCCTGAGCGGCCAGAAAGTGGCCGATTCTCCACTGGAAGCCAG
CAAGAGATGGGCCTTTAGAACCGGCGTGCCACCTAAGAACGTCGAGTACA
CAGAGGGCGAAGAGGCCAAGACCTGCTACAACATCAGCGTGACCGATCCT
AGCGGCAAGAGCCTGCTGCTGGACCCTCCTAGCAACATCAGAGACTACCC
CAAGTGCAAGACCGTGCACCACATCCAGGGACAGAATCCCCATGCTCAGG
GAATTGCCCTGCACCTGTGGGGCGCCTTTTTCCTGTATGATCGGATCGCC
TCCACCACCATGTACAGAGGCAAAGTGTTCACCGAGGGCAATATCGCCGC
CATGATCGTGAACAAGACAGTGCACAAGATGATCTTCAGCCGGCAAGGCC
AGGGCTACAGACACATGAATCTGACCAGCACCAACAAGTACTGGACCAGC
AGCAACGGCACCCAGACCAATGATACAGGCTGCTTTGGCGCCCTGCAAGA
GTACAACAGCACCAAGAATCAGACATGCGCCCCTAGCAAGATCCCTCCTC
CACTGCCTACTGCCAGACCTGAGATCAAGCCTACCAGCACACCTACCGAC
GCCACCAAGCTGAACACCACCGATCCAAACAGCGACGACGAGGATCTGAC
AACAAGCGGATCTGGCTCTGGCGAGCAAGAGCCATACACCACCTCTGATG
CCGTGACAAAGCAGGGCCTGAGCAGCACAATGCCTCCAACACCTTCTCCA
CAGCCTAGCACACCTCAGCAAGGCGGCAACAACACAAATCACTCTCAGGG
CGCCGTGACCGAGCCTGACAAGACAAATACCACAGCTCAGCCCAGCATGC
CTCCTCACAACACCACCACAATCTCCACCAACAACACCAGCAAGCACAAC
TTCAGCACACTGAGCGCCCCTCTCCAGAATACCACCAACTACAATACCCA
GAGCACCGCCACCGAGAACGAGCAGACATCTGCCCCTTCTAAGACCACAC
TGCCACCTACCGGCAATCCTACCACCGCCAAGAGCACCAATAGCACAAAG
GGCCCTACCACCACCGCTCCTAACACCACAAATGGCCACTTCACAAGCCC
AAGTCCTACACCTAACAGCACAACCCAGCACCTGGTGTACTTCAGACGGA
AGCGGAGCATCCTTTGGCGCGAGGGCGATATGTTCCCTTTCCTGGACGGC
CTGATCAACACCGAGATCGACTTCGACCCCATTCCAAACACCGAAACCAT
CTTCGACGAGAGCCCCAGCTTCAACACCTCCACCAATGAGGAACAGCACA
CCCCTCCAAACATCTCCCTGACCTTCAGCTACTTCCCCGACAAGAACGGC
GATACAGCCTACAGCGGCGAGAATGAGAATGACTGCGACGCCGAGCTGCG
GATTTGGAGCGTTCAAGAGGATGATCTGGCTGCCGGCCTGAGCTGGATCC
CTTTTTTTGGACCTGGCATCGAGGGCCTGTACACCGCCGGACTGATCAAG
AACCAGAACAACCTCGTGTGCAGACTGCGGAGACTGGCCAATCAGACCGC
CAAGTCTCTGGAACTGCTGCTGCGCGTGACCACCGAGGAAAGAACCTTCT
CTCTGATCAACCGGCACGCCATCGATTTTCTGCTGACCAGATGGGGCGGC
ACCTGTAAAGTTCTGGGCCCTGATTGCTGCATCGGAATCGAGGACCTGAG
CAAGAACATCTCCGAGCAGATCGACAAGATCCGCAAGGACGAGCAGAAAG
AGGAAACAGGCTGGGGACTCGGCGGCAAGTGGTGGACATCTGATTGGGGC
GTGCTGACCAATCTGGGAATCCTGCTGCTCCTGTCTATCGCCGTGCTGAT
CGCCCTGAGCTGCATCTGCCGGATCTTCACCAAGTACATCGGCTGA
Amino acid sequence encoded by unoptimised and
gene-optimised sequences (SEQ ID NO: 9):
MKTIYFLISLILIQSIKTLPVLEIASNSQPQDVDSVCSGTLQKTEDVHLM
GFTLSGQKVADSPLEASKRWAFRTGVPPKNVEYTEGEEAKTCYNISVTDP
SGKSLLLDPPSNIRDYPKCKTVHHIQGQNPHAQGIALHLWGAFFLYDRIA
STTMYRGKVFTEGNIAAMIVNKTVHKMIFSRQGQGYRHMNLTSTNKYWTS
SNGTQTNDTGCFGALQEYNSTKNQTCAPSKIPPPLPTARPEIKPTSTPTD
ATKLNTTDPNSDDEDLTTSGSGSGEQEPYTTSDAVTKQGLSSTMPPTPSP
QPSTPQQGGNNTNHSQGAVTEPDKTNTTAQPSMPPHNTTTISTNNTSKHN
FSTLSAPLQNTTNYNTQSTATENEQTSAPSKTTLPPTGNPTTAKSTNSTK
GPTTTAPNTTNGHFTSPSPTPNSTTQHLVYFRRKRSILWREGDMFPFLDG
LINTEIDFDPIPNTETIFDESPSFNTSTNEEQHTPPNISLTFSYFPDKNG
DTAYSGENENDCDAELRIWSVQEDDLAAGLSWIPFFGPGIEGLYTAGLIK
NQNNLVCRLRRLANQTAKSLELLLRVTTEERTFSLINRHAIDFLLTRWGG
TCKVLGPDCCIGIEDLSKNISEQIDKIRKDEQKEETGWGLGGKWWTSDWG
VLTNLGILLLLSIAVLIALSCICRIFTKYIG

### Example 4

| Tier 2-4 (SUDV anc -MLD) | |
|---|---|
| Sudan ebolavirus ancestral sequences with delete (minus “-”) mucin-like domain | |
| Nucleotide sequence (SEQ ID NO: 10): | |
| atgggaggac tgtctctgct gcaactgccc cgggacaagt tccggaagtc cagcttcttc | 60 |
| gtgtgggtca tcatcctgtt ccagaaagcc ttcagcatgc ccctgggcgt cgtgaccaat | 120 |
| agcacactgg aagtgaccga gatcgaccag ctcgtgtgca aggatcacct ggccagcacc | 180 |
| gatcagctga agtctgtggg actgaatctg gaaggcagcg gcgtgtccac agatatccct | 240 |
| agcgccacca agagatgggg ctttagaagc ggagtgcctc ctaaggtggt gtcttatgaa | 300 |
| gccggcgagt gggccgagaa ctgctacaac ctggaaatca agaagcccga cggcagcgag | 360 |
| tgtctgcctc ctccacctga tggcgtcaga ggcttcccta gatgcagata cgtgcacaag | 420 |
| gcccaaggca caggaccctg tcctggcgat tacgcctttc acaaggacgg cgcctttttc | 480 |
| ctgtacgatc ggctggcctc caccgtgatc tacagaggcg ttaactttgc cgagggcgtg | 540 |
| atcgccttcc tgatcctggc caagcctaaa gagacattcc tgcaaagccc tccaatccgc | 600 |
| gaggccgtga actacacaga gaacaccagc agctactacg ccaccagcta cctggaatac | 660 |
| gagatcgaga atttcggcgc ccagcacagc accacactgt tcaagatcga caacaacacc | 720 |
| ttcgtgcggc tggacagacc ccacacacct cagtttctgt tccagctgaa cgacaccatc | 780 |
| catctgcatc agcagctgag caacaccacc ggcagactga tttggaccct ggacgccaac | 840 |
| atcaacgccg acattggaga gtgggccttt tgggagaaca agaagaacct gagcgaacag | 900 |
| ctgagaggcg aggaactgag ctttgaggcc ctgtctctga ccaccgccgt gaaaacagtg | 960 |
| ctgcctcaag agtccaccag caacggcctg atcacaagca cagtgacagg catcctgggc | 1020 |
| agcctgggcc tgagaaaaag gtccagacgg caagtgaata ccaaggccac cggcaagtgc | 1080 |
| aaccccaacc tgcactattg gacagcccaa gagcagcaca atgccgccgg aatcgcctgg | 1140 |
| attccttatt ttggacctgg cgccgagggc atctataccg agggactgat gcacaaccag | 1200 |
| aacgccctcg tgtgtggact gagacagctg gccaatgaga caacacaggc cctccagctg | 1260 |
| tttctgagag ccaccaccga gctgagaacc tacaccatcc tgaaccggaa ggccatcgac | 1320 |
| tttctgctga gaagatgggg cggcacctgt agaatcctgg gacctgattg ctgcatcgag | 1380 |
| ccccacgact ggaccaagaa catcaccgac aagatcaacc agatcatcca cgacttcatc | 1440 |
| gacaaccctc tgcctaacca ggacaacgac gacaattggt ggacaggctg gcggcagtgg | 1500 |
| attcctgccg gaattggcat caccggcatc atcattgcca ttatcgccct gctgtgtgtg | 1560 |
| tgcaagctgc tgtgttga | 1578 |
| Amino acid sequence (SEQ ID NO: 11): | |
| MGGLSLLQLPRDKFRKSSFFVWVIILFQKAFSMPLGVVTNSTLEVTEIDQ | 50 |
| LVCKDHLASTDQLKSVGLNLEGSGVSTDIPSATKRWGFRSGVPPKVVSYE | 100 |
| AGEWAENCYNLEIKKPDGSECLPPPPDGVRGFPRCRYVHKAQGTGPCPGD | 150 |
| YAFHKDGAFFLYDRLASTVIYRGVNFAEGVIAFLILAKPKETFLQSPPIR | 200 |
| EAVNYTENTSSYYATSYLEYEIENFGAQHSTTLFKIDNNTFVRLDRPHTP | 250 |
| QFLFQLNDTIHLHQQLSNTTGRLIWTLDANINADIGEWAFWENKKNLSEQ | 300 |
| LRGEELSFEALSLTTAVKTVLPQESTSNGLITSTVTGILGSLGLRKRSRR | 350 |
| QVNTKATGKCNPNLHYWTAQEQHNAAGIAWIPYFGPGAEGIYTEGLMHNQ | 400 |
| NALVCGLRQLANETTQALQLFLRATTELRTYTILNRKAIDFLLRRWGGTC | 450 |
| RILGPDCCIEPHDWTKNITDKINQIIHDFIDNPLPNQDNDDNWWTGWRQW | 500 |
| IPAGIGITGIIIAIIALLCVCKLLC* | |

### Example 5

| Tier 2-6 (SUDV_EBOV-TAFV-BDBV_anc_-MLD) | |
|---|---|
| Ancesstral sequence to the four species Sudan, Zaire, Tai Forest, and Bundibugyo | |
| ebolavirus with the mucin-like-domain deleted. | |
| Nucleotide sequence (SEQ ID NO: 12): | |
| atgggcggag gatctagact gctgcaactg cccagagagc ggttcagaaa gaccagcttc | 60 |
| ttcgtgtggg tcatcatcct gttccagaaa gccttcagca tgcccctggg cgtcgtgacc | 120 |
| aatagcaccc tgaaagtgac cgagatcgac cagctcgtgt gcagagataa gctgagcagc | 180 |
| accagccagc tgaagtccgt gggactgaat ctggaaggca atggcgtggc cacagatgtg | 240 |
| cctagcgcca ccaaaagatg gggctttaga agcggcgtgc cacctaaggt ggtgtcttat | 300 |
| gaagccggcg agtgggccga gaactgctac aacctggaaa tcaagaagcc cgacggcagc | 360 |
| gagtgtctgc ctcctccacc tgatggcgtc agaggcttcc ctagatgcag atacgtgcac | 420 |
| aaggtgcaag gcacaggccc ctgtcctggc gatttcgcct ttcacaagga cggcgccttt | 480 |
| ttcctgtacg atcggctggc ctccaccgtg atctacagag gcacaacatt tgccgaaggc | 540 |
| gtggtggcct tcctgatcct gcctaagcct aagaaggact tctttcagag ccctcctatc | 600 |
| cgcgagcctg tgaacacaac agaggacccc agcagctact acaccaccag cacactgagc | 660 |
| tacgagatcg ataacttcgg cgccaacaag accaagacac tgttcaaggt ggacaaccac | 720 |
| acctacgtgc agctggacag accccacaca cctcagtttc tggtgcagct gaacgagaca | 780 |
| atccacacca acaacagact gagcaacacc accggcaggc tgatctggac cctggatcct | 840 |
| aagatcgaca ccgacatcgg agagtgggcc ttttgggaga acaagaagaa cttcagcaag | 900 |
| cagctgagag gcgaggaact gagctttaag gccctgagca ccaagacagg cgccaacgct | 960 |
| gtggataccg atgagtctag caagcccggc ctgatcacca acacagttag aggcgttgcc | 1020 |
| gacctgctga gcccttggag aagaaagcgg agacaagtga accccaatac caccaacaag | 1080 |
| tgcaacccta acctgcacta ctggacagcc caggatgaag gcgctgctgt tggactggcc | 1140 |
| tggattcctt attttggacc tgccgccgag ggcatctaca cagagggaat catgcacaac | 1200 |
| cagaatggcc tgatctgcgg cctgagacag ctggccaatg agacaacaca ggccctccag | 1260 |
| ctgtttctga gagccaccac cgagctgaga acctacagca tcctgaaccg gaaggccatc | 1320 |
| gactttctgc tgcaaagatg gggaggcacc tgtagaatcc tgggacctga ttgctgcatc | 1380 |
| gagccccacg actggaccaa gaacatcacc gacaagatca accagatcat ccacgacttc | 1440 |
| atcgacaacc ctctgcctga ccaggacgac gacgataatt ggtggacagg atggcggcag | 1500 |
| tggattcctg ccggaatcgg aatcacaggc gtgatcattg ccattatcgc cctgctgtgc | 1560 |
| atctgcaagt ttctgtgctg a | 1581 |
| Amino acid sequence (SEQ ID NO: 13): | |
| MGGGSRLLQLPRERFRKTSFFVWVIILFQKAFSMPLGVVTNSTLKVTEID | 50 |
| QLVCRDKLSSTSQLKSVGLNLEGNGVATDVPSATKRWGFRSGVPPKVVSY | 100 |
| EAGEWAENCYNLEIKKPDGSECLPPPPDGVRGFPRCRYVHKVQGTGPCPG | 150 |
| DFAFHKDGAFFLYDRLASTVIYRGTTFAEGVVAFLILPKPKKDFFQSPPI | 200 |
| REPVNTTEDPSSYYTTSTLSYEIDNFGANKTKTLFKVDNHTYVQLDRPHT | 250 |
| PQFLVQLNETIHTNNRLSNTTGRLIWTLDPKIDTDIGEWAFWENKKNFSK | 300 |
| QLRGEELSFKALSTKTGANAVDTDESSKPGLITNTVRGVADLLSPWRRKR | 350 |
| RQVNPNTTNKCNPNLHYWTAQDEGAAVGLAWIPYFGPAAEGIYTEGIMHN | 400 |
| QNGLICGLRQLANETTQALQLFLRATTELRTYSILNRKAIDFLLQRWGGT | 450 |
| CRILGPDCCIEPHDWTKNITDKINQIIHDFIDNPLPDQDDDDNWWTGWRQ | 500 |
| WIPAGIGITGVIIAIIALLCICKFLC* | |

### Example 6

| Tier 2-11 (RAVV-MARV_anc) | |
|---|---|
| Ancestral sequence to the strains Marburg Virus and Ravn Virus | |
| Nucleotide sequence (SEQ ID NO: 14): | |
| atgaagacca tctactttct gatcagcctg atcctgatcc agagcatcaa gaccctgcct | 60 |
| gtgctggaaa tcgccagcaa cagtcagccc caggatgtgg atagcgtgtg tagcggcacc | 120 |
| ctccagaaaa ccgaggatgt gcacctgatg ggctttaccc tgagcggcca gaaagtggcc | 180 |
| gattctccac tggaagccag caagagatgg gcctttagaa ccggcgtgcc acctaagaac | 240 |
| gtcgagtaca cagagggcga agaggccaag acctgctaca acatcagcgt gaccgatcct | 300 |
| agcggcaaga gcctgctgct ggaccctcct agcaacatca gagactaccc caagtgcaag | 360 |
| accgtgcacc acatccaggg acagaatccc catgctcagg gaattgccct gcacctgtgg | 420 |
| ggcgcctttt tcctgtatga tcggatcgcc tccaccacca tgtacagagg caaagtgttc | 480 |
| accgagggca atatcgccgc catgatcgtg aacaagacag tgcacaagat gatcttcagc | 540 |
| cggcaaggcc agggctacag acacatgaat ctgaccagca ccaacaagta ctggaccagc | 600 |
| agcaacggca cccagaccaa tgatacaggc tgctttggcg ccctgcaaga gtacaacagc | 660 |
| accaagaatc agacatgcgc ccctagcaag atccctcctc cactgcctac tgccagacct | 720 |
| gagatcaagc ctaccagcac acctaccgac gccaccaagc tgaacaccac cgatccaaac | 780 |
| agcgacgacg aggatctgac aacaagcgga tctggctctg gcgagcaaga gccatacacc | 840 |
| acctctgatg ccgtgacaaa gcagggcctg agcagcacaa tgcctccaac accttctcca | 900 |
| cagcctagca cacctcagca aggcggcaac aacacaaatc actctcaggg cgccgtgacc | 960 |
| gagcctgaca agacaaatac cacagctcag cccagcatgc ctcctcacaa caccaccaca | 1020 |
| atctccacca acaacaccag caagcacaac ttcagcacac tgagcgcccc tctccagaat | 1080 |
| accaccaact acaataccca gagcaccgcc accgagaacg agcagacatc tgccccttct | 1140 |
| aagaccacac tgccacctac cggcaatcct accaccgcca agagcaccaa tagcacaaag | 1200 |
| ggccctacca ccaccgctcc taacaccaca aatggccact tcacaagccc aagtcctaca | 1260 |
| cctaacagca caacccagca cctggtgtac ttcagacgga agcggagcat cctttggcgc | 1320 |
| gagggcgata tgttcccttt cctggacggc ctgatcaaca ccgagatcga cttcgacccc | 1380 |
| attccaaaca ccgaaaccat cttcgacgag agccccagct tcaacacctc caccaatgag | 1440 |
| gaacagcaca cccctccaaa catctccctg accttcagct acttccccga caagaacggc | 1500 |
| gatacagcct acagcggcga gaatgagaat gactgcgacg ccgagctgcg gatttggagc | 1560 |
| gttcaagagg atgatctggc tgccggcctg agctggatcc ctttttttgg acctggcatc | 1620 |
| gagggcctgt acaccgccgg actgatcaag aaccagaaca acctcgtgtg cagactgcgg | 1680 |
| agactggcca atcagaccgc caagtctctg gaactgctgc tgcgcgtgac caccgaggaa | 1740 |
| agaaccttct ctctgatcaa ccggcacgcc atcgattttc tgctgaccag atggggcggc | 1800 |
| acctgtaaag ttctgggccc tgattgctgc atcggaatcg aggacctgag caagaacatc | 1860 |
| tccgagcaga tcgacaagat ccgcaaggac gagcagaaag aggaaacagg ctggggactc | 1920 |
| ggcggcaagt ggtggacatc tgattggggc gtgctgacca atctgggaat cctgctgctc | 1980 |
| ctgtctatcg ccgtgctgat cgccctgagc tgcatctgcc ggatcttcac caagtacatc | 2040 |
| ggctga | 2046 |
| Amino acid sequence (SEQ ID NO: 15): | |
| MKTIYFLISLILIQSIKTLPVLEIASNSQPQDVDSVCSGTLQKTEDVHLM | 50 |
| GFTLSGQKVADSPLEASKRWAFRTGVPPKNVEYTEGEEAKTCYNISVTDP | 100 |
| SGKSLLLDPPSNIRDYPKCKTVHHIQGQNPHAQGIALHLWGAFFLYDRIA | 150 |
| STTMYRGKVFTEGNIAAMIVNKTVHKMIFSRQGQGYRHMNLTSTNKYWTS | 200 |
| SNGTQTNDTGCFGALQEYNSTKNQTCAPSKIPPPLPTARPEIKPTSTPTD | 250 |
| ATKLNTTDPNSDDEDLTTSGSGSGEQEPYTTSDAVTKQGLSSTMPPTPSP | 300 |
| QPSTPQQGGNNTNHSQGAVTEPDKTNTTAQPSMPPHNTTTISTNNTSKHN | 350 |
| FSTLSAPLQNTTNYNTQSTATENEQTSAPSKTTLPPTGNPTTAKSTNSTK | 400 |
| GPTTTAPNTTNGHFTSPSPTPNSTTQHLVYFRRKRSILWREGDMFPFLDG | 450 |
| LINTEIDFDPIPNTETIFDESPSFNTSTNEEQHTPPNISLTFSYFPDKNG | 500 |
| DTAYSGENENDCDAELRIWSVQEDDLAAGLSWIPFFGPGIEGLYTAGLIK | 550 |
| NQNNLVCRLRRLANQTAKSLELLLRVTTEERTFSLINRHAIDFLLTRWGG | 600 |
| TCKVLGPDCCIGIEDLSKNISEQIDKIRKDEQKEETGWGLGGKWWTSDWG | 650 |
| VLTNLGILLLLSIAVLIALSCICRIFTKYIG* | |

### Example 7

| pEVAC Expression Vector | |
|---|---|
| FIG. 3 shows a map of the pEVAC expression vector. The sequence of the multiple | |
| cloning site of the vector is given below, followed by its entire nucleotide sequence. | |
| Sequence of pEVAC Multiple Cloning Site (MCS) (SEQ ID NO: 16): | |
| | PstI KpnI SalI |
| pEVAC | 1301 ACAGACTGTT CCTTTCCATG GGTCTTTTCT GCAGTCACCG TCGGTACCGT |
| | BclI XbaI BamHI NotI BglII |
| Pevac | 1351 CGACACGTGT GATCATCTAG AGGATCCGCG GCCGCAGATC T |

Entire Sequence of pEVAC (SEQ ID NO: 17):

| | | |
|---|---|---|
| CMV-IE-E/P: | 248-989 | CMV immediate early 1 enhancer/promoter |
| KanR: | 3445-4098 | Kanamycin resistance |
| SD: | 990-1220 | Splice donor |
| SA: | 1221-1343 | Splice acceptor |
| Tbgh: | 1392-1942 | Terminator signal from bovine growth hormone |
| pUC-ori: | 2096-2769 | pUC-plasmid origin of replication |

| | |
|---|---|
| 1 | TCGCGCGTTT CGGTGATGAC GGTGAAAACC TCTGACACAT GCAGCTCCCG |
| 51 | GAGACGGTCA CAGCTTGTCT GTAAGCGGAT GCCGGGAGCA GACAAGCCCG |
| 101 | TCAGGGCGCG TCAGCGGGTG TTGGCGGGTG TCGGGGCTGG CTTAACTATG |
| 151 | CGGCATCAGA GCAGATTGTA CTGAGAGTGC ACCATATGCG GTGTGAAATA |
| 201 | CCGCACAGAT GCGTAAGGAG AAAATACCGC ATCAGATTGG CTATTGGCCA |
| 251 | TTGCATACGT TGTATCCATA TCATAATATG TACATTTATA TTGGCTCATG |
| 301 | TCCAACATTA CCGCCATGTT GACATTGATT ATTGACTAGT TATTAATAGT |
| 351 | AATCAATTAC GGGGTCATTA GTTCATAGCC CATATATGGA GTTCCGCGTT |
| 401 | ACATAACTTA CGGTAAATGG CCCGCCTGGC TGACCGCCCA ACGACCCCCG |
| 451 | CCCATTGACG TCAATAATGA CGTATGTTCC CATAGTAACG CCAATAGGGA |
| 501 | CTTTCCATTG ACGTCAATGG GTGGAGTATT TACGGTAAAC TGCCCACTTG |
| 551 | GCAGTACATC AAGTGTATCA TATGCCAAGT ACGCCCCCTA TTGACGTCAA |
| 601 | TGACGGTAAA TGGCCCGCCT GGCATTATGC CCAGTACATG ACCTTATGGG |
| 651 | ACTTTCCTAC TTGGCAGTAC ATCTACGTAT TAGTCATCGC TATTACCATG |
| 701 | GTGATGCGGT TTTGGCAGTA CATCAATGGG CGTGGATAGC GGTTTGACTC |
| 751 | ACGGGGATTT CCAAGTCTCC ACCCCATTGA CGTCAATGGG AGTTTGTTTT |
| 801 | GGCACCAAAA TCAACGGGAC TTTCCAAAAT GTCGTAACAA CTCCGCCCCA |
| 851 | TTGACGCAAA TGGGCGGTAG GCGTGTACGG TGGGAGGTCT ATATAAGCAG |
| 901 | AGCTCGTTTA GTGAACCGTC AGATCGCCTG GAGACGCCAT CCACGCTGTT |
| 951 | TTGACCTCCA TAGAAGACAC CGGGACCGAT CCAGCCTCCA TCGGCTCGCA |
| 1001 | TCTCTCCTTC ACGCGCCCGC CGCCCTACCT GAGGCCGCCA TCCACGCCGG |
| 1051 | TTGAGTCGCG TTCTGCCGCC TCCCGCCTGT GGTGCCTCCT GAACTGCGTC |
| 1101 | CGCCGTCTAG GTAAGTTTAA AGCTCAGGTC GAGACCGGGC CTTTGTCCGG |
| 1151 | CGCTCCCTTG GAGCCTACCT AGACTCAGCC GGCTCTCCAC GCTTTGCCTG |
| 1201 | ACCCTGCTTG CTCAACTCTA GTTAACGGTG GAGGGCAGTG TAGTCTGAGC |
| 1251 | AGTACTCGTT GCTGCCGCGC GCGCCACCAG ACATAATAGC TGACAGACTA |
| 1301 | ACAGACTGTT CCTTTCCATG GGTCTTTTCT GCAGTCACCG TCGGTACCGT |
| 1351 | CGACACGTGT GATCATCTAG AGGATCCGCG GCCGCAGATC TGCTGTGCCT |
| 1401 | TCTAGTTGCC AGCCATCTGT TGTTTGCCCC TCCCCCGTGC CTTCCTTGAC |
| 1451 | CCTGGAAGGT GCCACTCCCA CTGTCCTTTC CTAATAAAAT GAGGAAATTG |
| 1501 | CATCGCATTG TCTGAGTAGG TGTCATTCTA TTCTGGGGGG TGGGGTGGGG |
| 1551 | CAGGACAGCA AGGGGGAGGA TTGGGAAGAC AATAGCAGGC ATGCTGGGGA |
| 1601 | TGCGGTGGGC TCTATGGCTA CCCAGGTGCT GAAGAATTGA CCCGGTTCCT |
| 1651 | CCTGGGCCAG AAAGAAGCAG GCACATCCCC TTCTCTGTGA CACACCCTGT |
| 1701 | CCACGCCCCT GGTTCTTAGT TCCAGCCCCA CTCATAGGAC ACTCATAGCT |
| 1751 | CAGGAGGGCT CCGCCTTCAA TCCCACCCGC TAAAGTACTT GGAGCGGTCT |
| 1801 | CTCCCTCCCT CATCAGCCCA CCAAACCAAA CCTAGCCTCC AAGAGTGGGA |
| 1851 | AGAAATTAAA GCAAGATAGG CTATTAAGTG CAGAGGGAGA GAAAATGCCT |
| 1901 | CCAACATGTG AGGAAGTAAT GAGAGAAATC ATAGAATTTT AAGGCCATGA |
| 1951 | TTTAAGGCCA TCATGGCCTT AATCTTCCGC TTCCTCGCTC ACTGACTCGC |
| 2001 | TGCGCTCGGT CGTTCGGCTG CGGCGAGCGG TATCAGCTCA CTCAAAGGCG |
| 2051 | GTAATACGGT TATCCACAGA ATCAGGGGAT AACGCAGGAA AGAACATGTG |
| 2101 | AGCAAAAGGC CAGCAAAAGG CCAGGAACCG TAAAAAGGCC GCGTTGCTGG |
| 2151 | CGTTTTTCCA TAGGCTCCGC CCCCCTGACG AGCATCACAA AAATCGACGC |
| 2201 | TCAAGTCAGA GGTGGCGAAA CCCGACAGGA CTATAAAGAT ACCAGGCGTT |
| 2251 | TCCCCCTGGA AGCTCCCTCG TGCGCTCTCC TGTTCCGACC CTGCCGCTTA |
| 2301 | CCGGATACCT GTCCGCCTTT CTCCCTTCGG GAAGCGTGGC GCTTTCTCAT |
| 2351 | AGCTCACGCT GTAGGTATCT CAGTTCGGTG TAGGTCGTTC GCTCCAAGCT |
| 2401 | GGGCTGTGTG CACGAACCCC CCGTTCAGCC CGACCGCTGC GCCTTATCCG |
| 2451 | GTAACTATCG TCTTGAGTCC AACCCGGTAA GACACGACTT ATCGCCACTG |
| 2501 | GCAGCAGCCA CTGGTAACAG GATTAGCAGA GCGAGGTATG TAGGCGGTGC |
| 2551 | TACAGAGTTC TTGAAGTGGT GGCCTAACTA CGGCTACACT AGAAGAACAG |
| 2601 | TATTTGGTAT CTGCGCTCTG CTGAAGCCAG TTACCTTCGG AAAAAGAGTT |
| 2651 | GGTAGCTCTT GATCCGGCAA ACAAACCACC GCTGGTAGCG GTGGTTTTTT |
| 2701 | TGTTTGCAAG CAGCAGATTA CGCGCAGAAA AAAAGGATCT CAAGAAGATC |
| 2751 | CTTTGATCTT TTCTACGGGG TCTGACGCTC AGTGGAACGA AAACTCACGT |
| 2801 | TAAGGGATTT TGGTCATGAG ATTATCAAAA AGGATCTTCA CCTAGATCCT |
| 2851 | TTTAAATTAA AAATGAAGTT TTAAATCAAT CTAAAGTATA TATGAGTAAA |
| 2901 | CTTGGTCTGA CAGTTACCAA TGCTTAATCA GTGAGGCACC TATCTCAGCG |
| 2951 | ATCTGTCTAT TTCGTTCATC CATAGTTGCC TGACTCGGGG GGGGGGGGCG |
| 3001 | CTGAGGTCTG CCTCGTGAAG AAGGTGTTGC TGACTCATAC CAGGCCTGAA |
| 3051 | TCGCCCCATC ATCCAGCCAG AAAGTGAGGG AGCCACGGTT GATGAGAGCT |
| 3101 | TTGTTGTAGG TGGACCAGTT GGTGATTTTG AACTTTTGCT TTGCCACGGA |
| 3151 | ACGGTCTGCG TTGTCGGGAA GATGCGTGAT CTGATCCTTC AACTCAGCAA |
| 3201 | AAGTTCGATT TATTCAACAA AGCCGCCGTC CCGTCAAGTC AGCGTAATGC |
| 3251 | TCTGCCAGTG TTACAACCAA TTAACCAATT CTGATTAGAA AAACTCATCG |
| 3301 | AGCATCAAAT GAAACTGCAA TTTATTCATA TCAGGATTAT CAATACCATA |
| 3351 | TTTTTGAAAA AGCCGTTTCT GTAATGAAGG AGAAAACTCA CCGAGGCAGT |
| 3401 | TCCATAGGAT GGCAAGATCC TGGTATCGGT CTGCGATTCC GACTCGTCCA |
| 3451 | ACATCAATAC AACCTATTAA TTTCCCCTCG TCAAAAATAA GGTTATCAAG |
| 3501 | TGAGAAATCA CCATGAGTGA CGACTGAATC CGGTGAGAAT GGCAAAAGCT |
| 3551 | TATGCATTTC TTTCCAGACT TGTTCAACAG GCCAGCCATT ACGCTCGTCA |
| 3601 | TCAAAATCAC TCGCATCAAC CAAACCGTTA TTCATTCGTG ATTGCGCCTG |
| 3651 | AGCGAGACGA AATACGCGAT CGCTGTTAAA AGGACAATTA CAAACAGGAA |
| 3701 | TCGAATGCAA CCGGCGCAGG AACACTGCCA GCGCATCAAC AATATTTTCA |
| 3751 | CCTGAATCAG GATATTCTTC TAATACCTGG AATGCTGTTT TCCCGGGGAT |
| 3801 | CGCAGTGGTG AGTAACCATG CATCATCAGG AGTACGGATA AAATGCTTGA |
| 3851 | TGGTCGGAAG AGGCATAAAT TCCGTCAGCC AGTTTAGTCT GACCATCTCA |
| 3901 | TCTGTAACAT CATTGGCAAC GCTACCTTTG CCATGTTTCA GAAACAACTC |
| 3951 | TGGCGCATCG GGCTTCCCAT ACAATCGATA GATTGTCGCA CCTGATTGCC |
| 4001 | CGACATTATC GCGAGCCCAT TTATACCCAT ATAAATCAGC ATCCATGTTG |
| 4051 | GAATTTAATC GCGGCCTCGA GCAAGACGTT TCCCGTTGAA TATGGCTCAT |
| 4101 | AACACCCCTT GTATTACTGT TTATGTAAGC AGACAGTTTT ATTGTTCATG |
| 4151 | ATGATATATT TTTATCTTGT GCAATGTAAC ATCAGAGATT TTGAGACACA |
| 4201 | ACGTGGCTTT CCCCCCCCCC CCATTATTGA AGCATTTATC AGGGTTATTG |
| 4251 | TCTCATGAGC GGATACATAT TTGAATGTAT TTAGAAAAAT AAACAAATAG |
| 4301 | GGGTTCCGCG CACATTTCCC CGAAAAGTGC CACCTGACGT CTAAGAAACC |
| 4351 | ATTATTATCA TGACATTAAC CTATAAAAAT AGGCGTATCA CGAGGCCCTT |
| 4401 | TCGTC |

### Example 8

### Lead Candidate Optimized Antigenic Ebola Polypeptides Able to Induce a Broadly Neutralizing Antibody Response

There was a significant interest to develop vaccines against Ebola followed the West African outbreak in 2014. Programmes currently in clinical development have so far taken a ‘classical’ approach to vaccine development using Ebola and/or Marburg virus surface glycoproteins (GPs) from one to three strains expressed in a viral vector backbone. Antigen specificity comes only from the included EBOV strains: for example Merck use a GP from Kikwit; GSK use Mayinga EBOV and Gulu SUDV strains; Crucell and Profectus Biosiences both use a Marburg virus together with Zaire and Sudan Ebola strains; with the Novavax approach being unique in using the 2014 Makona EBOV strain.

Table 1 below shows flow cytometric assay results illustrating the strength of antibody binding to target antigens, representative of all Ebola virus species (subtypes) and Marburg viruses. Strength of binding is indicated by the heat-map where red (the darkest shading when viewed in grayscale) is very strong binding, decreasing through orange to yellow (progressively lighter shading when viewed in grayscale) and no binding/equal to negative control values are white. Serum samples 1-22 were taken from individuals immunised with other Ebola virus vaccine candidates. T2-4 and T2-6 are nucleic acid vaccines encoding lead candidate optimized antigenic Ebola polypeptide, combined with T2-11 a Marburg candidate, at pre-clinical stage testing with serum samples taken from immunised guinea pigs.

### Example 9

### Protection Achieved by a Trivalent Lassa, Ebola and Marburg Viral Vaccine (Tri-LEMvac) in an Ebola Challenge Model

We have developed a trivalent vaccine (Tri-LEMvac) that generates combined vaccine efficacy against future outbreaks of variants of the haemorrhagic fever Lassa, Ebola and Marburg viruses.

We have bioinformatically designed synthetic glycoprotein sequences from the GPC open reading frames of LASV (L) as well as EBOV (E) and MARV (M) from all available Arenavirus and Filovirus databases. These conserved sequences consist of neutralising antibody and T-cell rich epitopes for each of these viruses. To ensure that these synthetically designed LASV, EBOV and MARV envelopes were functional and antigenic, they were expressed as pseudotypes and quality controlled for both binding and neutralisation against a panel of broadly neutralising antibodies. Herein, we chose the vaccine derived vector Modified Vaccinia Ankara (MVA) for construction of the trimeric LEM vaccine.

The Modified Vaccinia Ankara (MVA) vaccine platform is a non-replicating strain (i.e. non-replicating in human cells), third generation smallpox vaccine and one of the most advanced recombinant poxviral vaccine vectors in human clinical trials (Cottingham & Carroll, Vaccine, 2013, 31 (39): 4247-51). MVA is a robust vector system capable of co-expressing up to four transgenes facilitating potent promoters and stable insertion sites (Orubu et al, Pone, 2012,7 (6) e0040167). MVA was chosen because: 1) its significant capacity to stably express multiple independent ORFs via compatible expression cassettes with strong and timely regulated promotors for trivalent LEM vaccination in one cost effective vaccine lot; 2) its ability to induce robust B and T-cell immune responses in animals and humans especially when primed or boosted with DNA or RNA vectors; and 3) vaccine lots can be thermally stabilised for storage and transport in developing countries in the absence of cold chain (Frey et al, Vaccine, 2015, 33 (39): 5225-34). Proof of principle for the Trivalent vaccine candidate has been demonstrated by: i) cassette validation for independent L, E and M GPC expression and epitope presentation; and ii) preclinical efficacy by Filovirus challenge. The challenge study results are shown in FIG. 4. The Ebola challenge model was lethal for non-vaccinated guinea pigs (Group 1, lower line) whereas all vaccinated guinea pigs (Group 2, upper line) were protected (left) and continued to gain weight (right).

### Example 10

### Pseudotype Virus Neutralisation Assay

FIG. 5 shows the results of a pseudotype virus neutralisation assay illustrating the strength of neutralising antibody responses to target antigens expressed on the surface of a pseudotyped virus, representative of all Ebola virus species and Marburg viruses. Strength of neutralisation is indicated by the heat-map where red (darkest shading when viewed in grayscale) is very strong neutralisation, decreasing through orange to yellow (progressively lighter shading when viewed in grayscale) and no neutralising/equal to negative control values are white.

T2-4 and T2-6 are nucleic acid vaccines each encoding lead candidate optimized antigenic Ebola polypeptide, combined with T2-11 a Marburg candidate, at pre-clinical stage testing with serum samples taken from immunised guinea pigs.

The results show that administering a combination of T2-6 and T2-11 vaccine inserts gave a synergistic increase in the breadth of the immune response.

### Example 11

### Antibody Binding Assay

FIG. 6 shows the results of an antibody binding assay. Antibody binding was measured by incubation of two groups of cells bearing two different group 1 influenza A glycoproteins on their surface (H1 pandemic and seasonal) with pooled mouse serum. Any bound antibodies were then detected by a secondary antibody, and results recorded using a flow cytometer. Binding was significantly increased before and after vaccination with all constructs, but not after vaccination with PBS (control). Overall, a DIOS vaccine candidate out-performed those from COBRA in both cases (*).

### Example 12

### Comparison of Immune Responses Induced by Two Different Computational Approaches

Four groups of six mice were immunized five times, at two-week intervals, with 25 μg of four separate pEVAC plasmids encoding HA gene antigens that were designed either by a method according to an embodiment of the invention (DIOS) or by a conventional method (COBRA).

Antibody-based FACS was carried out on cells expressing two different group 1 influenza A glycoproteins on their cell surface (seasonal H1N1, and pandemic origin H1N1). These were used to test mouse sera from animals immunized with either the COBRA or DIOS HA gene antigens. The results are shown in FIG. 7.

Overall, the DIOS HA gene antigens matched or significantly out-performed the COBRA HA gene antigens (**p<0.01, ***p<0.001).

### Example 13

### Cross-HA-Group Binding, and Pseudotype Neutralization of H7N9 (A/Shanghai2/2013)

We tested whether the DIOS-H1N1pdm vaccine of Example 12 (which produced higher levels of antibody binding than H1N1-COBRA to the pandemic H1 HA antigen) could evoke antibodies that recognize and bind divergent group 2 virus HA, such as that from pandemic potential H7N9 strain A/Shanghai/2/2013.

FIG. 8 shows the results of cross-HA-group binding (left panel), and pseudotype neutralization (right) of H7N9 (A/Shanghai2/2013), by sera from DIOS or COBRA DNA immunized mice. H7 binding data (left), confirmed by pseudotype neutralization data (right), shows that H1N1pdm-vaccinated mice showed the highest neutralization compared to the other groups. Significantly more binding was elicited by the DIOS-H1N1pdm vaccine than other groups tested, and was comparable with positive control broadly neutralizing monoclonal antibodies F16 (Corti et al., 2011, supra) and CR9114 (Dreyfus et al, Science, 2012; 337 (6100): 1343-1348).

These results support a conclusion that the DIOS-H1N1pdm immunogen cross neutralizes H7, and that cross-HA group immune protection is possible with vaccines produced by methods of the invention.

### Example 14

### Lassa Virus Glycoprotein

This example describes Lassa virus glycoprotein ancestral sequence produced using a method according to an embodiment of the invention, and modifications to the ancestral sequence to improve its immunogenicity by stabilising the structure.

Lassa fever is a hemorrhagic disease caused by an Old World (OW) arenavirus known as Lassa virus (LASV). The virus was first isolated in Nigeria in 1969 and is currently endemic in West Africa. Due to the high morbidity and mortality associated with Lassa hemorrhagic fever, LASV is classified as a category A pathogen.

assa virus is an enveloped ambisense RNA virus with a bisegmented genome. Viral particles are covered in mature glycoprotein (GP) trimeric spikes, which mediate viral entry. Like other class 1 viral fusion proteins, the envelope glycoprotein precursor (GPC) is translated as a single polypeptide and is proteolytically cleaved into three subunits. Processing occurs first in the endoplasmic reticulum (ER) by a cellular signal peptidase. GPC is then trafficked to the cis-Golgi apparatus and processed by cellular proprotein convertase subtilisin kexin isozyme-1/site-1 protease (SKI-1/S1P) to produce a noncovalent stable-signal peptide (SSP)/GP1/GP2 heterotrimer. Unlike other class I fusion proteins, the relatively long signal peptide of GPC is not degraded; it serves a chaperone-like function necessary for the correct trafficking and processing of GP. SSP interacts with the cytoplasmic domain of GP2 and is involved in pH sensing. GP1 is responsible for binding to cellular receptors, while GP2 mediates membrane fusion during viral entry.

Lassa virus glycoprotein ancestral sequence to lineages III and IV (L-10) (construct 1) was produced using a method according to an embodiment of the invention. Modifications were then introduced independently into the parental ancestral sequence (construct 1) to provide: (A) SOSEP (construct 2); and (B) FLEP (construct 4), as well as in combination with a glycan knock-out, called NtoK (to provide constructs 3 and 5), to stabilize the otherwise flexible heterotrimers and prevent dissociation of the external domain of the glycoprotein from the non-covalently linked transmembrane domain.
A) Two cystein residues were introduced at positions 207 and 360 to allow formation of a disulfide bridge (SOS) between the exterior and the transmembrane domains of GP. To facilitate complete cleavage of these two domains, the furin cleavage site was modified from RRLL to RRRR at position 256-259. Mutation of glutamate to proline at position 329 (EP) prevents structural rearrangements making the protein less flexible.
(B) The furin cleavage site (256-RRLL-259) between the C-terminus of the external domain and the N-terminus of the transmembrane domain was replaced by a flexible linker with the sequence 256-GGGGSGGGGS-265. Additionally, the EP-mutation as in (A) was introduced at position 335.

Variants of both designs were generated that additionally contain an asparagine to lysine mutation at position 272 or 278, for SOSEP-NtoK or FLEP-NtoK, respectively, to inactivate a glycosylation motif. Glycans at this position might block access of some neutralizing antibodies, such as 37.7H.

### Construct 1:

| Lassa virus glycoprotein ancestral sequence to lineages III and IV | |
|---|---|
| (L-10 = LASV_III_IV_anc) | |
| Amino acid sequence (SEQ ID NO: 18): | |
| MGQIVTFFQEVPHVIEEVMNIVLIALSLLAILKGLYNVATCGLIGLVTFL | 50 |
| LLCGRSCSTTLYKGVYELQTLELNMETLNMTMPLSCTKNNSHHYIRVGNE | 100 |
| TGLELTLTNTSIINHKFCNLSDAHKKNLYDHALMSIISTFHLSIPNFNQY | 150 |
| EAMSCDFNGGKISVQYNLSHSYAVDAANHCGTVANGVLQTFMRMAWGGSY | 200 |
| IALDSGRGNWDCIMTSYQYLIIQNTTWEDHCQFSRPSPIGYLGLLSQRTR | 250 |
| DIYISRRLLGTFTWTLSDSEGNETPGGYCLTRWMLIEAELKCFGNTAVAK | 300 |
| CNEKHDEEFCDMLRLFDFNKQAIRRLKAEAQMSIQLINKAVNALINDQLI | 350 |
| MKNHLRDIMGIPYCNYSKYWYLNHTITGKTSLPKCWLVSNGSYLNETHFS | 400 |
| DDIEQQADNMITEMLQKEYMDRQGKTPLGLVDLFVFSTSFYLISIFLHLV | 450 |
| KIPTHRHIVGKPCPKPHRLNHMGICSCGLYKQPGVPVRWKR* | |

```
DNA sequence (SEQ ID NO: 19):
      1 ATGGGCCAGA TCGTGACATT CTTCCAAGAG GTGCCCCACG TGATCGAAGA
    51 AGTGATGAAC ATCGTCCTGA TCGCCCTGAG CCTGCTGGCC ATCCTGAAGG
  101 GCCTGTATAA TGTGGCCACC TGTGGCCTGA TCGGCCTGGT CACATTTCTG
  151 CTGCTGTGCG GCAGAAGCTG CTCCACCACA CTGTATAAGG GCGTGTACGA
  201 GCTGCAAACC CTGGAACTGA ACATGGAAAC CCTGAACATG ACCATGCCTC
  251 TGAGCTGCAC CAAGAACAAC AGCCACCACT ACATCAGAGT GGGCAACGAG
  301 ACAGGCCTCG AGCTGACCCT GACCAACACC AGCATCATCA ACCACAAGTT
  351 CTGCAACCTG AGCGACGCCC ACAAGAAGAA CCTGTACGAT CACGCCCTGA
  401 TGAGCATCAT CTCCACCTTC CACCTGAGCA TCCCCAACTT CAACCAGTAC
  451 GAGGCCATGA GCTGCGACTT CAACGGCGGA AAGATCAGCG TGCAGTACAA
  501 TCTGAGCCAC AGCTATGCCG TGGACGCCGC CAATCATTGT GGAACAGTGG
  551 CCAATGGCGT GCTCCAGACC TTCATGAGAA TGGCCTGGGG CGGCAGCTAT
  601 ATCGCCCTGG ATTCTGGCAG AGGCAACTGG GACTGCATCA TGACCAGCTA
  651 CCAGTACCTG ATCATCCAGA ACACCACCTG GGAAGATCAC TGCCAGTTCA
  701 GCAGACCCTC TCCTATCGGA TACCTGGGCC TGCTGTCCCA GAGAACCCGG
  751 GACATCTACA TCTCTAGACG GCTGCTGGGC ACCTTCACCT GGACACTGTC
  801 TGATAGCGAG GGCAATGAGA CACCTGGCGG CTACTGTCTG ACCCGGTGGA
  851 TGCTGATTGA GGCCGAGCTG AAGTGCTTCG GAAATACCGC CGTGGCCAAG
  901 TGCAACGAGA AGCACGACGA GGAATTCTGC GACATGCTGC GGCTGTTCGA
  951 TTTCAACAAG CAGGCCATCA GACGGCTGAA GGCCGAGGCT CAGATGTCCA
1001 TCCAGCTGAT CAACAAGGCC GTGAATGCCC TGATTAACGA CCAGCTCATC
1051 ATGAAGAACC ACCTCAGGGA CATCATGGGC ATCCCTTACT GCAACTACAG
1101 CAAGTACTGG TATCTGAACC ACACCATCAC CGGCAAGACC AGCCTGCCTA
1151 AGTGCTGGCT GGTGTCCAAC GGCAGCTACC TGAACGAGAC ACACTTCAGC
1201 GACGACATCG AGCAGCAGGC CGACAACATG ATCACCGAGA TGCTCCAGAA
1251 AGAGTACATG GACCGGCAGG GCAAGACACC TCTGGGCCTT GTGGATCTGT
1301 TCGTGTTCAG CACCAGCTTC TACCTGATCT CTATCTTCCT GCACCTGGTC
1351 AAGATCCCCA CACACAGACA CATCGTGGGC AAGCCCTGTC CTAAGCCTCA
1401 CAGACTGAAC CATATGGGCA TCTGTAGCTG CGGCCTGTAC AAACAGCCTG
1451 GCGTGCCAGT GCGGTGGAAG AGATAA
```

### Construct 2:

| SOSEP-variant of construct 1 (L-10-SOSEP) | |
|---|---|
| Amino acid sequence (SEQ ID NO: 20): | |
| MGQIVTFFQEVPHVIEEVMNIVLIALSLLAILKGLYNVATCGLIGLVTFL | 50 |
| LLCGRSCSTTLYKGVYELQTLELNMETLNMTMPLSCTKNNSHHYIRVGNE | 100 |
| TGLELTLTNTSIINHKFCNLSDAHKKNLYDHALMSIISTFHLSIPNFNQY | 150 |
| EAMSCDFNGGKISVQYNLSHSYAVDAANHCGTVANGVLQTFMRMAWGGSY | 200 |
| IALDSGCGNWDCIMTSYQYLIIQNTTWEDHCQFSRPSPIGYLGLLSQRTR | 250 |
| DIYISRRRRGTFTWTLSDSEGNETPGGYCLTRWMLIEAELKCFGNTAVAK | 300 |
| CNEKHDEEFCDMLRLFDFNKQAIRRLKAPAQMSIQLINKAVNALINDQLI | 350 |
| MKNHLRDIMCIPYCNYSKYWYLNHTITGKTSLPKCWLVSNGSYLNETHFS | 400 |
| DDIEQQADNMITEMLQKEYMDRQGKTPLGLVDLFVFSTSFYLISIFLHLV | 450 |
| KIPTHRHIVGKPCPKPHRLNHMGICSCGLYKQPGVPVRWKR* | |

```
DNA-sequence (SEQ ID NO: 21):
      1 ATGGGCCAGA TCGTGACATT CTTCCAAGAG GTGCCCCACG TGATCGAAGA
    51 AGTGATGAAC ATCGTCCTGA TCGCCCTGAG CCTGCTGGCC ATCCTGAAGG
  101 GCCTGTATAA TGTGGCCACC TGTGGCCTGA TCGGCCTGGT CACATTTCTG
  151 CTGCTGTGCG GCAGAAGCTG CTCCACCACA CTGTATAAGG GCGTGTACGA
  201 GCTGCAAACC CTGGAACTGA ACATGGAAAC CCTGAACATG ACCATGCCTC
  251 TGAGCTGCAC CAAGAACAAC AGCCACCACT ACATCAGAGT GGGCAACGAG
  301 ACAGGCCTCG AGCTGACCCT GACCAACACC AGCATCATCA ACCACAAGTT
  351 CTGCAACCTG AGCGACGCCC ACAAGAAGAA CCTGTACGAT CACGCCCTGA
  401 TGAGCATCAT CTCCACCTTC CACCTGAGCA TCCCCAACTT CAACCAGTAC
  451 GAGGCCATGA GCTGCGACTT CAACGGCGGA AAGATCAGCG TGCAGTACAA
  501 TCTGAGCCAC AGCTATGCCG TGGACGCCGC CAATCATTGT GGAACAGTGG
  551 CCAATGGCGT GCTCCAGACC TTCATGAGAA TGGCCTGGGG CGGCAGCTAT
  601 ATCGCCCTGG ATTCTGGCTG TGGCAACTGG GACTGCATCA TGACCAGCTA
  651 CCAGTACCTG ATCATCCAGA ACACCACCTG GGAAGATCAC TGCCAGTTCA
  701 GCAGACCCTC TCCTATCGGA TACCTGGGCC TGCTGTCCCA GAGAACCCGG
  751 GACATCTACA TCTCTCGGCG GAGAAGAGGC ACCTTCACCT GGACACTGTC
  801 TGATAGCGAG GGCAATGAGA CACCTGGCGG CTACTGTCTG ACCCGGTGGA
  851 TGCTGATTGA GGCCGAGCTG AAGTGCTTCG GAAATACCGC CGTGGCCAAG
  901 TGCAACGAGA AGCACGACGA GGAATTCTGC GACATGCTGC GGCTGTTCGA
  951 TTTCAACAAG CAGGCCATCA GACGGCTGAA GGCCCCTGCT CAGATGTCCA
1001 TCCAGCTGAT CAACAAGGCC GTGAATGCCC TGATTAACGA CCAGCTCATC
1051 ATGAAGAACC ACCTCAGGGA CATCATGTGC ATCCCTTACT GCAACTACAG
1101 CAAGTACTGG TATCTGAACC ACACCATCAC CGGCAAGACC AGCCTGCCTA
1151 AGTGCTGGCT GGTGTCCAAC GGCAGCTACC TGAACGAGAC ACACTTCAGC
1201 GACGACATCG AGCAGCAGGC CGACAACATG ATCACCGAGA TGCTCCAGAA
1251 AGAGTACATG GACCGGCAGG GCAAGACACC TCTGGGCCTT GTGGATCTGT
1301 TCGTGTTCAG CACCAGCTTC TACCTGATCT CTATCTTCCT GCACCTGGTC
1351 AAGATCCCCA CACACAGACA CATCGTGGGC AAGCCCTGTC CTAAGCCTCA
1401 CAGACTGAAC CATATGGGCA TCTGTAGCTG CGGCCTGTAC AAACAGCCTG
1451 GCGTGCCAGT GCGGTGGAAG AGATAA
```

### Construct 3:

| SOSEP-variant of construct 1 with N-to-K-mutation (L-10-SOSEP-NtoK) | |
|---|---|
| Amino acid sequence (SEQ ID NO: 22): | |
| MGQIVTFFQEVPHVIEEVMNIVLIALSLLAILKGLYNVATCGLIGLVTFL | 50 |
| LLCGRSCSTTLYKGVYELQTLELNMETLNMTMPLSCTKNNSHHYIRVGNE | 100 |
| TGLELTLTNTSIINHKFCNLSDAHKKNLYDHALMSIISTFHLSIPNFNQY | 150 |
| EAMSCDFNGGKISVQYNLSHSYAVDAANHCGTVANGVLQTFMRMAWGGSY | 200 |
| IALDSGCGNWDCIMTSYQYLIIQNTTWEDHCQFSRPSPIGYLGLLSQRTR | 250 |
| DIYISRRRRGTFTWTLSDSEGKETPGGYCLTRWMLIEAELKCFGNTAVAK | 300 |
| CNEKHDEEFCDMLRLFDFNKQAIRRLKAPAQMSIQLINKAVNALINDQLI | 350 |
| MKNHLRDIMCIPYCNYSKYWYLNHTITGKTSLPKCWLVSNGSYLNETHFS | 400 |
| DDIEQQADNMITEMLQKEYMDRQGKTPLGLVDLFVFSTSFYLISIFLHLV | 450 |
| KIPTHRHIVGKPCPKPHRLNHMGICSCGLYKQPGVPVRWKR* | |

```
DNA-sequence (SEQ ID NO: 23):
      1 ATGGGCCAGA TCGTGACATT CTTCCAAGAG GTGCCCCACG TGATCGAAGA
    51 AGTGATGAAC ATCGTCCTGA TCGCCCTGAG CCTGCTGGCC ATCCTGAAGG
  101 GCCTGTATAA TGTGGCCACC TGTGGCCTGA TCGGCCTGGT CACATTTCTG
  151 CTGCTGTGCG GCAGAAGCTG CTCCACCACA CTGTATAAGG GCGTGTACGA
  201 GCTGCAAACC CTGGAACTGA ACATGGAAAC CCTGAACATG ACCATGCCTC
  251 TGAGCTGCAC CAAGAACAAC AGCCACCACT ACATCAGAGT GGGCAACGAG
  301 ACAGGCCTCG AGCTGACCCT GACCAACACC AGCATCATCA ACCACAAGTT
  351 CTGCAACCTG AGCGACGCCC ACAAGAAGAA CCTGTACGAT CACGCCCTGA
  401 TGAGCATCAT CTCCACCTTC CACCTGAGCA TCCCCAACTT CAACCAGTAC
  451 GAGGCCATGA GCTGCGACTT CAACGGCGGA AAGATCAGCG TGCAGTACAA
  501 TCTGAGCCAC AGCTATGCCG TGGACGCCGC CAATCATTGT GGAACAGTGG
  551 CCAATGGCGT GCTCCAGACC TTCATGAGAA TGGCCTGGGG CGGCAGCTAT
  601 ATCGCCCTGG ATTCTGGCTG TGGCAACTGG GACTGCATCA TGACCAGCTA
  651 CCAGTACCTG ATCATCCAGA ACACCACCTG GGAAGATCAC TGCCAGTTCA
  701 GCAGACCCTC TCCTATCGGA TACCTGGGCC TGCTGTCCCA GAGAACCCGG
  751 GACATCTACA TCTCTCGGCG GAGAAGAGGC ACCTTCACCT GGACACTGTC
  801 TGATAGCGAG GGCAAAGAGA CACCTGGCGG CTACTGTCTG ACCCGGTGGA
  851 TGCTGATTGA GGCCGAGCTG AAGTGCTTCG GAAATACCGC CGTGGCCAAG
  901 TGCAACGAGA AGCACGACGA GGAATTCTGC GACATGCTGC GGCTGTTCGA
  951 TTTCAACAAG CAGGCCATCA GACGGCTGAA GGCCCCTGCT CAGATGTCCA
1001 TCCAGCTGAT CAACAAGGCC GTGAATGCCC TGATTAACGA CCAGCTCATC
1051 ATGAAGAACC ACCTCAGGGA CATCATGTGC ATCCCTTACT GCAACTACAG
1101 CAAGTACTGG TATCTGAACC ACACCATCAC CGGCAAGACC AGCCTGCCTA
1151 AGTGCTGGCT GGTGTCCAAC GGCAGCTACC TGAACGAGAC ACACTTCAGC
1201 GACGACATCG AGCAGCAGGC CGACAACATG ATCACCGAGA TGCTCCAGAA
1251 AGAGTACATG GACCGGCAGG GCAAGACACC TCTGGGCCTT GTGGATCTGT
1301 TCGTGTTCAG CACCAGCTTC TACCTGATCT CTATCTTCCT GCACCTGGTC
1351 AAGATCCCCA CACACAGACA CATCGTGGGC AAGCCCTGTC CTAAGCCTCA
1401 CAGACTGAAC CATATGGGCA TCTGTAGCTG CGGCCTGTAC AAACAGCCTG
1451 GCGTGCCAGT GCGGTGGAAG AGATAA
```

### Construct 4:

| FLEP-variant of construct 1 (L-10-FLEP) | |
|---|---|
| Amino acid sequence (SEQ ID NO: 24): | |
| MGQIVTFFQEVPHVIEEVMNIVLIALSLLAILKGLYNVATCGLIGLVTFL | 50 |
| LLCGRSCSTTLYKGVYELQTLELNMETLNMTMPLSCTKNNSHHYIRVGNE | 100 |
| TGLELTLTNTSIINHKFCNLSDAHKKNLYDHALMSIISTFHLSIPNFNQY | 150 |
| EAMSCDFNGGKISVQYNLSHSYAVDAANHCGTVANGVLQTFMRMAWGGSY | 200 |
| IALDSGRGNWDCIMTSYQYLIIQNTTWEDHCQFSRPSPIGYLGLLSQRTR | 250 |
| DIYISGGGGSGGGGSGTFTWTLSDSEGNETPGGYCLTRWMLIEAELKCFG | 300 |
| NTAVAKCNEKHDEEFCDMLRLFDFNKQAIRRLKAPAQMSIQLINKAVNAL | 350 |
| INDQLIMKNHLRDIMGIPYCNYSKYWYLNHTITGKTSLPKCWLVSNGSYL | 400 |
| NETHFSDDIEQQADNMITEMLQKEYMDRQGKTPLGLVDLFVFSTSFYLIS | 450 |
| IFLHLVKIPTHRHIVGKPCPKPHRLNHMGICSCGLYKQPGVPVRWKR* | |

```
DNA-sequence (SEQ ID NO: 25):
      1 ATGGGCCAGA TCGTGACATT CTTCCAAGAG GTGCCCCACG TGATCGAAGA
    51 AGTGATGAAC ATCGTCCTGA TCGCCCTGAG CCTGCTGGCC ATCCTGAAGG
  101 GCCTGTATAA TGTGGCCACC TGTGGCCTGA TCGGCCTGGT CACATTTCTG
  151 CTGCTGTGCG GCAGAAGCTG CTCCACCACA CTGTATAAGG GCGTGTACGA
  201 GCTGCAAACC CTGGAACTGA ACATGGAAAC CCTGAACATG ACCATGCCTC
  251 TGAGCTGCAC CAAGAACAAC AGCCACCACT ACATCAGAGT GGGCAACGAG
  301 ACAGGCCTCG AGCTGACCCT GACCAACACC AGCATCATCA ACCACAAGTT
  351 CTGCAACCTG AGCGACGCCC ACAAGAAGAA CCTGTACGAT CACGCCCTGA
  401 TGAGCATCAT CTCCACCTTC CACCTGAGCA TCCCCAACTT CAACCAGTAC
  451 GAGGCCATGA GCTGCGACTT CAACGGCGGA AAGATCAGCG TGCAGTACAA
  501 TCTGAGCCAC AGCTATGCCG TGGACGCCGC CAATCATTGT GGAACAGTGG
  551 CCAATGGCGT GCTCCAGACC TTCATGAGAA TGGCCTGGGG CGGCAGCTAT
  601 ATCGCCCTGG ATTCTGGCAG AGGCAACTGG GACTGCATCA TGACCAGCTA
  651 CCAGTACCTG ATCATCCAGA ACACCACCTG GGAAGATCAC TGCCAGTTCA
  701 GCAGACCCTC TCCTATCGGA TACCTGGGCC TGCTGTCCCA GAGAACCCGG
  751 GACATCTACA TCTCTGGCGG CGGAGGATCT GGCGGAGGTG GAAGTGGCAC
  801 CTTCACCTGG ACACTGTCTG ATAGCGAGGG CAATGAGACA CCTGGCGGCT
  851 ACTGTCTGAC CCGGTGGATG CTGATTGAGG CCGAGCTGAA GTGCTTCGGA
  901 AATACCGCCG TGGCCAAGTG CAACGAGAAG CACGACGAGG AATTCTGCGA
  951 CATGCTGCGG CTGTTCGATT TCAACAAGCA GGCCATCAGA CGGCTGAAGG
1001 CCCCTGCTCA GATGTCCATC CAGCTGATCA ACAAGGCCGT GAATGCCCTG
1051 ATTAACGACC AGCTCATCAT GAAGAACCAC CTCAGGGACA TCATGGGCAT
1101 CCCTTACTGC AACTACAGCA AGTACTGGTA TCTGAACCAC ACCATCACCG
1151 GCAAGACCAG CCTGCCTAAG TGCTGGCTGG TGTCCAACGG CAGCTACCTG
1201 AACGAGACAC ACTTCAGCGA CGACATCGAG CAGCAGGCCG ACAACATGAT
1251 CACCGAGATG CTCCAGAAAG AGTACATGGA CCGGCAGGGC AAGACACCTC
1301 TGGGCCTTGT GGATCTGTTC GTGTTCAGCA CCAGCTTCTA CCTGATCTCT
1351 ATCTTCCTGC ACCTGGTCAA GATCCCCACA CACAGACACA TCGTGGGCAA
1401 GCCCTGTCCT AAGCCTCACA GACTGAACCA TATGGGCATC TGTAGCTGCG
1451 GCCTGTACAA ACAGCCTGGC GTGCCAGTGC GGTGGAAGAG ATAA
```

### Construct 5:

| FLEP-variant of construt 1 with N-to-K-mutation (L-10-FLEP-NtoK) | |
|---|---|
| Amino acid sequence (SEQ ID NO: 26): | |
| MGQIVTFFQEVPHVIEEVMNIVLIALSLLAILKGLYNVATCGLIGLVTFL | 50 |
| LLCGRSCSTTLYKGVYELQTLELNMETLNMTMPLSCTKNNSHHYIRVGNE | 100 |
| TGLELTLTNTSIINHKFCNLSDAHKKNLYDHALMSIISTFHLSIPNFNQY | 150 |
| EAMSCDFNGGKISVQYNLSHSYAVDAANHCGTVANGVLQTFMRMAWGGSY | 200 |
| IALDSGRGNWDCIMTSYQYLIIQNTTWEDHCQFSRPSPIGYLGLLSQRTR | 250 |
| DIYISGGGGSGGGGSGTFTWTLSDSEGKETPGGYCLTRWMLIEAELKCFG | 300 |
| NTAVAKCNEKHDEEFCDMLRLFDFNKQAIRRLKAPAQMSIQLINKAVNAL | 350 |
| INDQLIMKNHLRDIMGIPYCNYSKYWYLNHTITGKTSLPKCWLVSNGSYL | 400 |
| NETHFSDDIEQQADNMITEMLQKEYMDRQGKTPLGLVDLFVFSTSFYLIS | 450 |
| IFLHLVKIPTHRHIVGKPCPKPHRLNHMGICSCGLYKQPGVPVRWKR* | |

```
DNA-sequence (SEQ ID NO: 27):
      1 ATGGGCCAGA TCGTGACATT CTTCCAAGAG GTGCCCCACG TGATCGAAGA
    51 AGTGATGAAC ATCGTCCTGA TCGCCCTGAG CCTGCTGGCC ATCCTGAAGG
  101 GCCTGTATAA TGTGGCCACC TGTGGCCTGA TCGGCCTGGT CACATTTCTG
  151 CTGCTGTGCG GCAGAAGCTG CTCCACCACA CTGTATAAGG GCGTGTACGA
  201 GCTGCAAACC CTGGAACTGA ACATGGAAAC CCTGAACATG ACCATGCCTC
  251 TGAGCTGCAC CAAGAACAAC AGCCACCACT ACATCAGAGT GGGCAACGAG
  301 ACAGGCCTCG AGCTGACCCT GACCAACACC AGCATCATCA ACCACAAGTT
  351 CTGCAACCTG AGCGACGCCC ACAAGAAGAA CCTGTACGAT CACGCCCTGA
  401 TGAGCATCAT CTCCACCTTC CACCTGAGCA TCCCCAACTT CAACCAGTAC
  451 GAGGCCATGA GCTGCGACTT CAACGGCGGA AAGATCAGCG TGCAGTACAA
  501 TCTGAGCCAC AGCTATGCCG TGGACGCCGC CAATCATTGT GGAACAGTGG
  551 CCAATGGCGT GCTCCAGACC TTCATGAGAA TGGCCTGGGG CGGCAGCTAT
  601 ATCGCCCTGG ATTCTGGCAG AGGCAACTGG GACTGCATCA TGACCAGCTA
  651 CCAGTACCTG ATCATCCAGA ACACCACCTG GGAAGATCAC TGCCAGTTCA
  701 GCAGACCCTC TCCTATCGGA TACCTGGGCC TGCTGTCCCA GAGAACCCGG
  751 GACATCTACA TCTCTGGCGG CGGAGGATCT GGCGGAGGTG GAAGTGGCAC
  801 CTTCACCTGG ACACTGTCTG ATAGCGAGGG CAAAGAGACA CCTGGCGGCT
  851 ACTGTCTGAC CCGGTGGATG CTGATTGAGG CCGAGCTGAA GTGCTTCGGA
  901 AATACCGCCG TGGCCAAGTG CAACGAGAAG CACGACGAGG AATTCTGCGA
  951 CATGCTGCGG CTGTTCGATT TCAACAAGCA GGCCATCAGA CGGCTGAAGG
1001 CCCCTGCTCA GATGTCCATC CAGCTGATCA ACAAGGCCGT GAATGCCCTG
1051 ATTAACGACC AGCTCATCAT GAAGAACCAC CTCAGGGACA TCATGGGCAT
1101 CCCTTACTGC AACTACAGCA AGTACTGGTA TCTGAACCAC ACCATCACCG
1151 GCAAGACCAG CCTGCCTAAG TGCTGGCTGG TGTCCAACGG CAGCTACCTG
1201 AACGAGACAC ACTTCAGCGA CGACATCGAG CAGCAGGCCG ACAACATGAT
1251 CACCGAGATG CTCCAGAAAG AGTACATGGA CCGGCAGGGC AAGACACCTC
1301 TGGGCCTTGT GGATCTGTTC GTGTTCAGCA CCAGCTTCTA CCTGATCTCT
1351 ATCTTCCTGC ACCTGGTCAA GATCCCCACA CACAGACACA TCGTGGGCAA
1401 GCCCTGTCCT AAGCCTCACA GACTGAACCA TATGGGCATC TGTAGCTGCG
1451 GCCTGTACAA ACAGCCTGGC GTGCCAGTGC GGTGGAAGAG ATAA
```

### Example 15

### Lassa Virus Nucleoprotein

This example describes Lassa virus nucleoprotein ancestral sequence produced using a method according to an embodiment of the invention.

### Construct 6:

| Lassa virus nucleoprotein ancestral sequence of Nigerian Lassa isolates | |
|---|---|
| (L-NP-1 - L=NP=CovAnc-1+N) | |
| Amino acid sequence (SEQ ID NO: 28): | |
| MSASKEVKSFLWTQSLRRELSGYCSNIKLQVVKDAQALLHGLDFSEVSNV | 50 |
| QRLMRKQKRDDSDLKRLRDLNQAVNNLVELKSTQQKSILRVGTLTSDDLL | 100 |
| TLAADLEKLKSKVIRTERPLSSGVYMGNLSTQQLEQRRALLNMIGMVGGA | 150 |
| QGTQPGRDGVVRVWDVKNPDLLNNQFGTMPSLTLACLTKQGQVDLNDAVL | 200 |
| ALTDLGLIYTAKYPNSSDLDRLSQSHPILNMVDTKKSSLNISGYNFSLGA | 250 |
| AVKAGACMLDGGNMLETIKVTPQTMDGILKSILKVKKSLGMFVSDTPGER | 300 |
| NPYENILYKICLSGDGWPYIASRTSIVGRAWENTTVDLESDGKPQKVGTA | 350 |
| GSNKSLQSAGFPTGLTYSQLMTLKDSMMQLDPSAKTWIDIEGRPEDPVEI | 400 |
| ALYQPMSGCYIHFFREPTDLKQFKQDAKYSHGIDVADLFPAQPGLTSAVI | 450 |
| EALPRNMVLTCQGSDDIKRLLDSQGRRDIKLIDIALSKADSRRFENAVWD | 500 |
| QCKDLCHMHTGVVVEKKKRGGKEEITPHCALMDCIMYDAAVSGGLNIPVL | 550 |
| RAVLPRDMVFRTSSPKVVL* | |

```
DNA-sequence (SEQ ID NO: 29):
      1 ATGAGCGCCA GCAAAGAAGT GAAAAGCTTC CTCTGGACCC AGAGCCTGCG
    51 GAGAGAGCTG TCTGGCTACT GCTCCAACAT CAAGCTCCAG GTGGTCAAGG
  101 ACGCCCAGGC TCTGCTGCAT GGCCTGGATT TCAGCGAGGT GTCCAACGTG
  151 CAGCGGCTGA TGAGAAAGCA GAAGCGGGAC GACAGCGACC TGAAGAGACT
  201 GAGGGATCTG AACCAGGCCG TGAACAACCT GGTGGAACTG AAGTCTACCC
  251 AGCAGAAATC CATCCTGAGA GTGGGCACCC TGACCAGCGA CGATCTGCTG
  301 ACACTGGCCG CCGATCTGGA AAAGCTGAAG TCCAAAGTGA TCCGGACCGA
  351 GAGGCCACTG TCTAGCGGAG TGTACATGGG CAACCTGAGC ACCCAGCAGC
  401 TGGAACAGAG AAGGGCCCTG CTGAACATGA TCGGCATGGT TGGAGGCGCC
  451 CAGGGAACAC AGCCTGGAAG AGATGGTGTC GTCAGAGTGT GGGACGTGAA
  501 GAACCCCGAC CTGCTCAACA ACCAGTTCGG CACCATGCCT TCTCTGACCC
  551 TGGCCTGCCT GACAAAGCAG GGCCAAGTGG ACCTGAACGA TGCCGTGCTG
  601 GCTCTGACTG ATCTGGGCCT GATCTACACC GCCAAGTATC CCAACAGCTC
  651 CGACCTGGAC AGGCTGAGCC AGTCTCACCC CATCCTGAAC ATGGTGGACA
  701 CCAAGAAGTC CAGCCTGAAC ATCAGCGGCT ACAACTTCTC TCTGGGCGCT
  751 GCCGTGAAAG CCGGCGCTTG TATGCTTGAC GGCGGCAACA TGCTGGAAAC
  801 CATCAAAGTG ACCCCTCAGA CCATGGACGG CATCCTGAAA AGTATCCTGA
  851 AAGTGAAGAA ATCCCTGGGC ATGTTCGTGT CCGACACACC CGGCGAGAGA
  901 AACCCCTACG AGAACATCCT GTACAAGATT TGCCTGAGCG GCGACGGCTG
  951 GCCCTATATC GCCAGCAGAA CATCTATCGT GGGCAGAGCT TGGGAGAACA
1001 CCACCGTGGA CCTGGAATCC GATGGCAAGC CTCAGAAAGT GGGCACAGCC
1051 GGCAGCAACA AGAGCCTCCA GTCTGCCGGA TTTCCTACCG GCCTGACATA
1101 CAGCCAGCTG ATGACCCTGA AGGACAGCAT GATGCAGCTG GACCCTAGCG
1151 CCAAGACCTG GATCGACATT GAGGGCAGAC CCGAGGATCC CGTGGAAATC
1201 GCTCTGTACC AGCCTATGAG CGGCTGCTAT ATCCACTTCT TCAGAGAGCC
1251 CACCGATCTG AAGCAGTTCA AGCAGGACGC CAAGTACAGC CACGGAATCG
1301 ACGTGGCCGA TCTGTTCCCA GCTCAGCCAG GACTGACATC CGCCGTGATT
1351 GAAGCCCTGC CTAGAAACAT GGTGCTGACC TGTCAGGGCA GCGACGACAT
1401 CAAGAGACTG CTGGACAGCC AGGGCAGAAG AGATATCAAG CTGATCGATA
1451 TCGCCCTGAG CAAGGCCGAC TCTCGGAGAT TCGAAAACGC CGTGTGGGAC
1501 CAGTGCAAGG ACCTGTGTCA CATGCACACA GGCGTGGTGG TGGAAAAGAA
1551 GAAGCGCGGA GGCAAAGAGG AAATCACCCC TCACTGCGCC CTGATGGACT
1601 GCATTATGTA TGACGCCGCC GTGTCTGGCG GCCTGAATAT CCCTGTTCTG
1651 AGAGCCGTGC TGCCCCGCGA CATGGTGTTT AGAACAAGCA GCCCCAAGGT
1701 GGTGCTCTGA
```

### Example 16

### Lassa Virus Nucleoprotein

This example describes Lassa virus nucleoprotein ancestral sequence produced using a method according to an embodiment of the invention.

### Construct 7:

| Lassa virus nucleoprotein ancestral sequence of Sierra Leone isolates | |
|---|---|
| (L-NP-1 = L=NP=CovAnc-2+SL) | |
| Amino acid sequence (SEQ ID NO: 30): | |
| MSASKEIKSFLWTQSLRRELSGYCSNIKLQVVKDAQALLHGLDFSEVSNV | 50 |
| QRLMRKERRDDNDLKRLRDLNQAVNNLVELKSTQQKSILRVGTLTSDDLL | 100 |
| ILAADLEKLKSKVTRTERPLSAGVYMGNLSSQQLDQRRALLNMIGMSGGN | 150 |
| QGARAGRDGVVRVWDVKNAELLNNQFGTMPSLTLACLTKQGQVDLNDAVQ | 200 |
| ALTDLGLIYTAKYPNTSDLDRLTQSHPILNMIDTKKSSLNISGYNFSLGA | 250 |
| AVKAGACMLDGGNMLETIKVSPQTMDGILKSILKVKKALGMFISDTPGER | 300 |
| NPYENILYKICLSGDGWPYIASRTSITGRAWENTVVDLESDGKPQKAGSN | 350 |
| NSNKSLQSAGFTAGLTYSQLMTLKDAMLQLDPNAKTWMDIEGRPEDPVEI | 400 |
| ALYQPSSGCYIHFFREPTDLKQFKQDAKYSHGIDVTDLFAAQPGLTSAVI | 450 |
| DALPRNMVITCQGSDDIRKLLESQGRKDIKLIDIALSKTDSRKYENAVWD | 500 |
| QYKDLCHMHTGVVVEKKKRGGKEEITPHCALMDCIMFDAAVSGGLNTSVL | 550 |
| RAVLPRDMVFRTSTPRVVL* | |

```
DNA-sequence (SEQ ID NO: 31):
      1 ATGAGCGCCA GCAAAGAGAT CAAGAGCTTC CTGTGGACCC AGAGCCTGCG
    51 GAGAGAGCTG TCTGGCTACT GCTCCAACAT CAAGCTCCAG GTGGTCAAGG
  101 ACGCCCAGGC TCTGCTGCAT GGCCTGGATT TCAGCGAGGT GTCCAACGTG
  151 CAGCGGCTGA TGCGGAAAGA GAGAAGGGAC GACAACGACC TGAAGCGGCT
  201 GAGGGATCTG AACCAGGCCG TGAACAACCT GGTGGAACTG AAGTCTACCC
  251 AGCAGAAATC CATCCTGAGA GTGGGCACCC TGACCAGCGA CGATCTGCTG
  301 ATTCTGGCCG CCGACCTGGA AAAGCTGAAG TCCAAAGTGA CCCGGACCGA
  351 GAGGCCACTG TCTGCTGGTG TCTACATGGG CAACCTGAGC AGCCAGCAGC
  401 TGGATCAGAG AAGGGCCCTG CTGAACATGA TCGGCATGAG CGGCGGAAAT
  451 CAGGGCGCTA GAGCTGGCAG AGATGGCGTC GTCAGAGTGT GGGACGTGAA
  501 GAATGCCGAG CTGCTCAACA ACCAGTTCGG CACCATGCCT AGCCTGACAC
  551 TGGCCTGCCT GACAAAGCAG GGCCAAGTGG ACCTGAACGA TGCTGTGCAG
  601 GCCCTGACTG ATCTGGGCCT GATCTACACC GCCAAGTATC CCAACACCAG
  651 CGACCTGGAC AGACTGACCC AGTCTCACCC CATCCTGAAT ATGATCGACA
  701 CCAAGAAGTC CAGCCTGAAC ATCAGCGGCT ACAACTTCTC TCTGGGCGCT
  751 GCCGTGAAAG CCGGCGCTTG TATGCTTGAC GGCGGCAACA TGCTGGAAAC
  801 CATCAAGGTG TCCCCACAGA CCATGGACGG CATCCTGAAA AGTATCCTGA
  851 AAGTGAAGAA AGCCCTGGGC ATGTTCATCA GCGACACCCC TGGCGAGAGA
  901 AACCCCTACG AGAACATCCT GTACAAGATT TGCCTGAGCG GCGACGGCTG
  951 GCCCTATATC GCCAGCAGAA CCAGCATTAC CGGCAGAGCT TGGGAGAACA
1001 CCGTGGTGGA TCTGGAAAGC GACGGCAAGC CTCAGAAGGC CGGCAGCAAC
1051 AACTCCAACA AGAGCCTCCA GTCCGCCGGC TTCACAGCCG GCCTGACATA
1101 TAGCCAGCTG ATGACCCTGA AGGACGCCAT GCTGCAACTG GACCCCAATG
1151 CCAAGACCTG GATGGACATC GAGGGCAGAC CTGAGGACCC TGTGGAAATC
1201 GCCCTGTACC AGCCTAGCTC CGGCTGCTAT ATCCACTTCT TCAGAGAGCC
1251 CACCGATCTG AAGCAGTTCA AGCAGGACGC CAAGTACAGC CACGGCATCG
1301 ACGTGACCGA TCTGTTTGCT GCTCAGCCCG GACTGACCTC CGCCGTGATT
1351 GATGCCCTGC CTCGGAACAT GGTCATCACC TGTCAGGGCA GCGACGACAT
1401 CCGGAAGCTG CTGGAATCTC AGGGCAGAAA GGATATCAAG CTGATCGATA
1451 TCGCCCTGAG CAAGACCGAC AGCCGGAAGT ACGAAAACGC CGTGTGGGAC
1501 CAGTACAAGG ACCTGTGCCA CATGCACACA GGCGTGGTGG TGGAAAAGAA
1551 GAAGCGCGGA GGCAAAGAGG AAATCACCCC TCACTGCGCT CTGATGGACT
1601 GCATCATGTT TGACGCCGCC GTGTCTGGCG GCCTGAATAC CTCTGTTCTG
1651 AGAGCCGTGC TGCCCAGAGA CATGGTGTTC AGAACAAGCA CCCCTAGAGT
1701 GGTGCTCTGA
```

1.-53. (canceled)

54. An isolated nucleic acid molecule, comprising a nucleic acid sequence that is:
i) at least 75%, 80%, 85%, 90%, 95%, 96%, 97%, 98%, or 99% identical with SEQ ID NO: 2, or identical with SEQ ID NO:2;
or
ii) at least 75%, 80%, 85%, 90%, 95%, 96%, 97%, 98%, or 99% identical with SEQ ID NO: 8, or identical with SEQ ID NO:8;
or the complement thereof.

55.-56. (canceled)

57. An isolated polypeptide, comprising an amino acid sequence that is:
i) at least 95%, 96%, 97%, 98%, or 99% identical with an amino acid sequence encoded by SEQ ID NO:2, or identical with the amino acid sequence encoded by SEQ ID NO:2; or
ii) at least 95%, 96%, 97%, 98%, or 99% identical with an amino acid sequence encoded by SEQ ID NO:8, or identical with the amino acid sequence encoded by SEQ ID NO:8.

58. An isolated polypeptide, comprising an amino acid sequence that is:
i) at least 95%, 96%, 97%, 98%, or 99% identical with SEQ ID NO:3, or identical with SEQ ID NO:3;
or
ii) at least 95%, 96%, 97%, 98%, or 99% identical with SEQ ID NO:9, or identical with SEQ ID NO:9.

59. (canceled)

60. An isolated nucleic acid of claim 54, wherein the nucleic acid is codon-optimized, optionally gene-optimized, for expression in mammalian cells.

61. An isolated nucleic acid encoding a polypeptide of claim 57, wherein the nucleic acid is codon-optimized, optionally gene-optimized, for expression in mammalian cells.

62. A vector comprising a nucleic acid of claim 54, optionally which further comprises a promoter operably linked to the nucleic acid, preferably wherein the promoter is for expression of a polypeptide encoded by the nucleic acid in mammalian cells.

63.-65. (canceled)

66. A vector according to claim 62, which is a vaccine vector, preferably which is a viral vaccine vector, a bacterial vaccine vector, an RNA vaccine vector, or a DNA vaccine vector.

67. (canceled)

68. An isolated cell comprising a vector of claim 62.

69. A pseudotyped virus particle comprising the polypeptide of claim 57 or 58.

70. A method of producing a pseudotyped virus particle of claim 69, which includes transfecting a host cell with a vector according to claim 62.

71. A fusion protein comprising a polypeptide according to claim 57.

72. A pharmaceutical composition comprising a nucleic acid according to claim 54, a vector according to claim 62, or a polypeptide according to claim 57, and a pharmaceutically acceptable carrier, excipient, or diluent.

73.-74. (canceled)

75. A pharmaceutical composition according to claim 72, which further comprises an adjuvant for enhancing an immune response in a subject to the polypeptide, or to a polypeptide encoded by the nucleic acid, of the composition.

76. A method of inducing an immune response to a virus of the Filoviridae family in a subject, which comprises administering to the subject a nucleic acid according to claim 54, a polypeptide according to claim 57 or 58, a vector according to claim 62, or a pharmaceutical composition according to claim 72.

77. A method of immunizing a subject against a virus of the Filoviridae family, which comprises administering to the subject a nucleic acid according to claim 54, a polypeptide according to claim 57 or 58, a vector according to claim 62, or a pharmaceutical composition according to claim 72.

78.-110. (canceled)

111. An isolated nucleic acid encoding a polypeptide of claim 58, wherein the nucleic acid is codon-optimized, optionally gene-optimized, for expression in mammalian cells.

112. A fusion protein comprising a polypeptide according to claim 58.

113. A pharmaceutical composition comprising a polypeptide according to claim 58, and a pharmaceutically acceptable carrier, excipient, or diluent.

114. A method of inducing an immune response to a virus of the Filoviridae family in a subject, which comprises administering to the subject a polypeptide according to claim 58.

115. A method of immunizing a subject against a virus of the Filoviridae family, which comprises administering to the subject a polypeptide according to claim 58.
